(19) United States
(12) Patent Application Publication
(10) Pub. No.: US 2025/0366981 A1
(43) Pub. Date: Dec. 4, 2025

(54) Corneal Inlay And Pinhole Lens Structure For Implantation Into A Cornea Of An Eye

(71) Applicant: Gholam A. Peyman, Sun City, AZ (US)

(72) Inventor: Gholam A. Peyman, Sun City, AZ (US)

(21) Appl. No.: 19/307,960

(22) Filed: Aug. 22, 2025

Related U.S. Application Data
(63) Continuation-in-part of application No. 19/074,412, filed on Mar. 9, 2025.
Application No. 19/074,412 is a continuation-in-part of application No. 19/047,492, filed on Feb. 6, 2025.
Application No. 19/047,492 is a continuation-in-part of application No. 18/406,148, filed on Jan. 6, 2024.
Application No. 18/406,148 is a continuation-in-part of application No. 18/205,320, filed on Jun. 2, 2023.
Application No. 18/205,320 is a continuation-in-part of application No. 17/683,344, filed on Feb. 28, 2022, now Pat. No. 12,383,393.
Application No. 17/683,344 is a continuation-in-part of application No. 16/927,882, filed on Jul. 13, 2020, now Pat. No. 11,259,914.
(60) Provisional application No. 63/026,033, filed on May 16, 2020.
(60) Provisional application No. 63/348,092, filed on Jun. 2, 2022.
(60) Provisional application No. 63/437,609, filed on Jan. 6, 2023.
(60) Provisional application No. 63/550,151, filed on Feb. 6, 2024.
(60) Provisional application No. 63/555,496, filed on Feb. 20, 2024.
(60) Provisional application No. 63/563,356, filed on Mar. 9, 2024.
(60) Provisional application No. 63/686,057, filed on Aug. 22, 2024.

(51) Int. Cl. A61F 2/14; A61L 27/26; A61L 27/36; C08L 27/16

(52) U.S. Cl. CPC A61F 2/1451; A61L 27/26; A61L 27/3604; C08L 27/16

(57) ABSTRACT

A corneal inlay and pinhole lens structure for implantation into a cornea of an eye is disclosed herein. In one embodiment, a corneal inlay for implantation into a cornea of an eye includes an inlay body formed from natural tissue of a donor cornea; and a central pinhole disposed in the inlay body, the central pinhole being surrounded by a darkened bounding wall. In another embodiment, a pinhole lens structure includes an insertable pinhole body defining a through pinhole of approximately 0.9-2.0 mm in diameter with a surrounding darkened wall having an approximately 0.1-1 mm wall thickness, the insertable pinhole body configured to be inserted into a corneal pocket in a cornea of an eye.

### CROSS-REFERENCE TO RELATED APPLICATIONS

This patent application claims priority to U.S. Provisional Patent Application No. 63/686,057, entitled “Tissue-Augmented Corneal Inlay Surgery Technique”, filed on Aug. 22, 2024, and is a continuation-in-part of application Ser. No. 19/074,412, entitled “Tissue-Augmented Corneal Inlay Surgery Technique”, filed on Mar. 9, 2025, which claims priority to U.S. Provisional Patent Application No. 63/563,356, entitled “Tissue-Augmented Corneal Inlay Surgery Technique”, filed on Mar. 9, 2024, and Ser. No. 19/074,412 is a continuation-in-part of application Ser. No. 19/047,492, entitled “Ablatable Corneal Inlay For Simultaneous Correction Of Refractive Errors And Presbyopia”, filed on Feb. 6, 2025, which claims priority to U.S. Provisional Patent Application No. 63/550,151, entitled “Ablatable Corneal Inlay For Simultaneous Correction Of Refractive Errors And Presbyopia”, filed on Feb. 6, 2024, and U.S. Provisional Patent Application No. 63/555,496, entitled “Ablatable Corneal Inlay For Simultaneous Correction Of Refractive Errors And Presbyopia”, filed on Feb. 20, 2024, and Ser. No. 19/047,492 is a continuation-in-part of application Ser. No. 18/406,148, entitled “Scleral Lens For Correction Of Keratoconus And/Or Presbyopia”, filed on Jan. 6, 2024, which claims priority to U.S. Provisional Patent Application No. 63/437,609, entitled “Scleral Lens For Correction Of Keratoconus And/Or Presbyopia”, filed on Jan. 6, 2023, and Ser. No. 18/406,148 is a continuation-in-part of application Ser. No. 18/205,320, entitled “Tissue-Augmented Corneal Inlay Surgery Technique”, filed on Jun. 2, 2023, which claims priority to U.S. Provisional Patent Application No. 63/348,092, entitled “Tissue-Augmented Corneal Inlay Surgery Technique”, filed on Jun. 2, 2022, and Ser. No. 18/205,320 is a continuation-in-part of application Ser. No. 17/683,344, entitled “Ablatable Corneal Inlay For Correction Of Refractive Errors And/Or Presbyopia”, filed on Feb. 28, 2022, now U.S. Pat. No. 12,383,393, and Ser. No. 17/683,344 is a continuation-in-part of application Ser. No. 16/927,882, entitled “Molding or 3-D Printing of a Synthetic Refractive Corneal Lenslet”, filed Jul. 13, 2020, now U.S. Pat. No. 11,259,914, which claims priority to U.S. Provisional Patent Application No. 63/026,033, entitled “Molding or 3-D Printing of a Synthetic Refractive Corneal Lenslet”, filed on May 16, 2020, the disclosure of each of which is hereby incorporated by reference as if set forth in their entirety herein.

### STATEMENT REGARDING FEDERALLY SPONSORED RESEARCH OR DEVELOPMENT

Not Applicable.

### NAMES OF THE PARTIES TO A JOINT RESEARCH AGREEMENT

### Not Applicable.

### INCORPORATION BY REFERENCE OF MATERIAL SUBMITTED ON A COMPACT DISK

Not Applicable.

### BACKGROUND OF THE INVENTION

### 1. Field of the Invention

The invention generally relates to a corneal inlay and pinhole lens structure for implantation into a cornea of an eye. The invention further relates to a tissue-augmented corneal inlay surgery technique where a corneal inlay is implanted under a corneal flap or in a pocket in order to supplement a thickness of the cornea. The invention yet further relates to an ablatable corneal inlay for simultaneous correction of refractive errors and presbyopia.

### 2. Background

Corneal scarring is a major cause of blindness, especially in developing countries. There are various causes for corneal scarring, which include: bacterial infections, viral infections, fungal infections, parasitic infections, genetic corneal problems, Fuch's dystrophy, and other corneal dystrophies. A corneal transplant is often required if the corneal scarring is extensive, and cannot be corrected by other means. However, there can be major complications associated with a corneal transplant, such as corneal graft rejection wherein the transplanted cornea is rejected by the patient's immune system.

A normal emmetropic eye includes a cornea, a lens and a retina. The cornea and lens of a normal eye cooperatively focus light entering the eye from a far point, i.e., infinity, onto the retina. However, an eye can have a disorder known as ametropia, which is the inability of the lens and cornea to focus the far point correctly on the retina. Typical types of ametropia are myopia, hypermetropia or hyperopia, and astigmatism.

A myopic eye has either an axial length that is longer than that of a normal emmetropic eye, or a cornea or lens having a refractive power stronger than that of the cornea and lens of an emmetropic eye. This stronger refractive power causes the far point to be projected in front of the retina.

Conversely, a hypermetropic or hyperopic eye has an axial length shorter than that of a normal emmetropic eye, or a lens or cornea having a refractive power less than that of a lens and cornea of an emmetropic eye. This lesser refractive power causes the far point to be focused behind the retina.

An eye suffering from astigmatism has a defect in the lens or shape of the cornea converting an image of the point of light to a line. Therefore, an astigmatic eye is incapable of sharply focusing images on the retina.

While laser surgical techniques, such as laser-assisted in situ keratomileusis (LASIK) and photorefractive keratectomy (PRK) are known for correcting refractive errors of the eye, these laser surgical techniques have complications, such as post-operative pain and dry eye. Also, these laser surgical techniques cannot be safely used on patients with corneas having certain biomechanical properties. For example, corneal ectasia may occur if these laser surgical techniques are applied to patients having thin corneas (e.g., corneas with thicknesses that are less than 500 microns).

Therefore, what is needed is a method for corneal transplantation that reduces the likelihood that the implanted cornea will be rejected by the patient. Moreover, a method is needed for corneal transplantation that is capable of preserving the clarity of the transplanted cornea. Furthermore, there is a need for a method of corneal transplantation that reduces the likelihood that the transplanted cornea will be invaded by migrating cells. Also, what is needed is a method for corneal lenslet implantation for modifying the cornea to better correct ametropic conditions. In addition, a method is needed for corneal lenslet implantation that prevents a lens implant from moving around inside the cornea once implanted so that the lens implant remains centered about the visual axis of the eye. Further, what is needed is a method for intracorneal lens implantation for modifying the cornea to better correct ametropic conditions.

In addition, numerous corneal diseases affect the clarity of the cornea necessitating partial or full thickness corneal replacement. These diseases are generally inherited affecting the cornea but no other organs. The disorders can involve one part of the cornea, but subsequently spread to the neighboring layers. Among the genetic disease involving corneal endothelial cells are Fuchs endothelial dystrophy, hereditary endothelial posterior polymorphic dystrophy, etc. causing damage to the corneal endothelial cells which prevent flooding of the cornea with aqueous fluid and producing the cloudiness of the normally transparent corneal stroma. Other genetic diseases involve the corneal stroma, such as granular corneal dystrophy, macular corneal dystrophy, Schneider crystalline dystrophy, and lattice corneal dystrophy, etc. all blocking or distorting the light that passes through the cornea on way to reach the sensory retina. Others conditions, such as keratoconus and keratoglobus, affect the mechanical stability of the cornea to resist the intraocular pressure. With time the cornea expands and can rupture without a surgical intervention of a corneal transplantation. The other genetic diseases affect the anterior layer of the cornea, the bowman layer of the cornea or the corneal epithelium, such as in Meesmann juvenile epithelial dystrophy, epithelial basement membrane dystrophy, gelatinous drop-like dystrophy, Lisch epithelial corneal dystrophy and Reis-Bucklers corneal dystrophy, and genetic recurrent corneal erosion. However, a number of other conditions can cause damage to the cornea, which results in losing its transparency, e.g., after, injuries, infections, corneal ulcers, or previous cataract surgeries or glaucoma surgeries.

At present about less than 200,000 corneal transplantations are performed each year in the world, but more than 12 million people are in need of corneal transplantation. This discrepancy is created by the need for surgery and unavailability of corneas for transplantation. Some of the reasons stem from the religious beliefs refusing another person's tissue, but most importantly, the retrieved human corneas from human eye banks can be stored only for a limited time which is at present is about 11 days. Even if only a part of the cornea is used for lamellar keratoplasty which requires the corneal stroma, the remaining part of the cornea must be discarded. The use of an animal cornea is not tolerated in humans. In addition, roughly about 10% of human corneal transplants can be rejected by the patients because of the incompatibility of the tissue.

Therefore, there is a further need to reduce the burden of corneal availability by producing synthetic corneal stromal lenslets that at least can be used for partial lamellar transplantation, in patients who have a limited corneal scared stroma after injury and infection. In addition, there is a need to address the growing need in refractive surgery to modify the refractive power of the cornea by a biocompatible refractive partial cornea or lenslet. Obtaining these corneas from the eye bank has been described in previous patents by the present inventor (see e.g., U.S. Pat. Nos. 10,314,690 and 10,583,221, which are hereby incorporated by reference as if set forth in their entirety herein). However, the need for refractive surgery is more than for corneal transplantation. Using the eye bank corneas for creating a lenslet would eliminate their badly needed indications described for patients that require them.

At present, over five million refractive surgeries are done in the world for myopia, hyperopia, astigmatism, keratoconus or keratoglobus eyes. Practically all presently available refractive procedures require ablating a part of the cornea or removing a part of the corneal stroma which thins out these corneas and can potentially lead to ectasia of the corneas, e.g., after the LASIK procedure, etc. leading to the need for a corneal transplantation.

Further, patients above the age of 45 years generally are not considered a candidate for corneal refractive surgery, such as LASIK or SMILE procedures. These two procedures remove a part of the corneal stroma with an excimer laser or femtosecond laser to correct the refractive errors of the eye defocus and astigmatism for the patient to see the far object without the use of glasses.

In young people below the age of 45, the crystalline lens of the eye has the ability to change its shape by ciliary muscles that contracts and relaxes the myriads of microns thick cords called zonules that are attached to the crystalline lens capsule and from another end to the ciliary muscle and suspend the crystalline lens behind the iris, in the posterior chamber of the eye. The circular contraction of the ciliary muscle loosens the zonules and as a result the crystalline lens becomes more convex. This process is called accommodation, by which the near object in front of the eye, such as a newspaper, becomes in focus for the retina to see the letters sharp for reading. This process enables the person to see any object from infinity to about 30 cm sharp as long as the crystalline lens is flexible. However, with aging, the crystalline lens becomes more rigid and the eye cannot accommodate to see the near objects sharp.

Since the standard LASIK and SMILE procedures do not correct presbyopia, the ophthalmologist normally recommends the patient wait until the lens becomes a cataract that can be removed and replaced with a multifocal intraocular lens (IOL), which to some degree, provides sharp images at focal points from the eye at various distances.

Though LASIK surgery for presbyopia can convert the refractive power of one eye to see near objects and the other eye to see far objects, the so-called monovision, it is not tolerated by most people and reduces, to some degree, the stereovision. The scleral-based surgery is another attempt to correct presbyopia but it is the least predictable.

Therefore, there is a further need for an ablatable corneal inlay that is capable of simultaneous correction of refractive errors and presbyopia.

The cornea is the transparent dome-shaped tissue of the eye that is exposed to the outside world. The external light coming from an object passes through the cornea, then through the crystalline lens before reaching the retina with its photoreceptors initiating biochemical responses that produces an electrical signal that goes through the optic nerve to the brain and ultimately reaches the visual cortex located in the back of the brain producing the sensation of vision of any object seen. The cornea has a diameter of about 12 mm in horizontal direction and 11 mm vertically. The corneal thickness increases from 500 microns centrally to >650 microns in the periphery. It has an index of refraction of about 1.37 and a curvature of 7.8 mm. The cornea is also the first structure in the eye that acts like a lens creating a dioptric power of about 43.00D. The cornea is made of five layers of tissue and cells. The outer layer of the cornea is composed of non-keratinized epithelial cells. The first layer of the epithelial cells are stratified having microvilli, which is covered with mucin and tear fluid, followed by winged epithelial cells and basal epithelial cells being in contact with the collagenous Bowman membrane separating them from the corneal stroma. The corneal stroma is made of lamellar collagen mostly type I, III, V, VI etc. collagen and keratocytes followed deeper in the corneal stroma by the Descemet Membrane that is made of type IV, VIII collagen and supports hexagonal endothelial cells that build a barrier to flow of aqueous fluid from the anterior chamber of the eye into the corneal stroma. The fluid has to pass through these cells before reaching the corneal stroma. If the endothelial cells are damaged, the uncontrolled aqueous flow causes the corneal stroma to swell and become cloudy and lose its transparency. The lamellar arrangement and the size of the collagen bundles contribute to the transparency of the cornea. The corneal stroma has a cellular component called keratocytes dispersed among the collagen layer that normally are transparent, but can respond to the corneal epithelial cell injury and its cytokines and become active losing their transparency or forming scar tissue that interferes with vision.

The cornea is endowed with numerous nerves that penetrate the corneal stroma building a sub-epithelial nerve plexus that penetrate the epithelial cells and render the cornea one of the most sensitive parts of the body. Damage to the corneal nerves causes the cornea to lose its sensation affecting a normal tear reflex so that the eye becomes dry with its subsequent side effects, such as inflammation or infection, etc.

The cornea contributes to the majority part of dioptric power needed for the external light to be focused on the retina. The crystalline lens contributes only 1/10% of the total dioptric power. Therefore, the majority of the refractive errors are caused by the corneal aberration. The cornea is also a structure that can be easily modified because of its accessibility without the need of entering the eye cavity, as is the case with all other intraocular surgery, e.g., when the crystalline lens has a cataract or is damaged by a trauma, etc.

Though attempts had been made to correct the shape of the cornea by mechanical means, such as using a knife in radial keratotomy, or the use of microkeratome to perform keratomileusis by freezing and milling the cornea, none gained widespread approval because of the serious damage to the corneal mechanics occurring in radial keratotomy or the difficulty of operation and impreciseness of freezing as a part of the cornea and milling it outside the body and replacing it subsequently over the cornea.

In 1980, Peyman tried ablating the cornea with a CO₂ laser in animals to find out if the laser could be used to correct refractive errors of the cornea. Unfortunately, the CO₂ laser damaged the corneal surface causing burns and scars. Subsequently, when an excimer laser became available, Peyman and his associates independently evaluated the effect of various excimer lasers on the cornea and found that the laser beam produced by argon fluoride ablated the cornea without burning it. In 1985, for the first time, Peyman filed a patent for a procedure that is now known as LASIK (Laser-assisted in situ keratomileusis, U.S. Pat. No. 4,840,175, which is hereby incorporated by reference as if set forth in its entirety herein) in which a corneal flap was created and corneal stromal ablation was done after exposure of the corneal stroma and the corneal flap was replaced over the treated area, contributing to rapid recovery of the vision.

In order for the inlays to be better tolerated inside the corneal pocket, Peyman developed a method for combining implantation of an inlay with the crosslinking of the surrounding corneal tissue to create a space that would not come in contact with the inlay to cause rejection or creating an immune privileged space. However, implantation of a corneal inlay, though tolerated by the body required some time for the visual acuity to recover. Since all inlays are produced without the corneal endothelial cells or a barrier to prevent rapid flow of fluid in the stroma area, this means that slight exposure of the inlay with a preservative fluid, etc. during the inlay storage or transfer, the inlay swells slightly and loses part of its transparency. Therefore, after implantation of an inlay, recovery of vision takes usually >1-2 weeks or more to become transparent or regain to its normal transparency. This is a long time for the patient to wait for his or her vision to fully recover and would make bilateral surgery not desirable.

Though the LASIK procedure is an accepted procedure, the FDA limited its use for eyes that need less than 7.00 D power that is equal to roughly removing an area of the stroma with the thickness of 70 microns. This decision was made because higher dioptric powers would thin the corneal thickness and could cause the cornea to bulge forward with time due to the intraocular pressure. Thus, the other limitation of LASIK is for corneas <450 micron thickness for the same reason. The refractive correction of higher dioptric errors can have the potential of creating ectasia by overly thinning a cornea which, at times, requires a keratoplasty that replaces the thin cornea with an eye bank eye or a synthetic polymeric cornea. In addition, children would not qualify because the eyes would grow with time and potentially need repeated surgery, which would thin out the cornea further and the operation is irreversible.

The implantation of an inlay and correction of refractive error would solve all these problems. However, as mentioned it would take some time for the inlay or the cornea to become completely transparent.

Therefore, there has been a need for a technology that provides all the benefits of ablating a corneal stroma, as is done with the LASIK procedure, but with a modified corneal inlay over the corneal stroma so as to create immediate transparency of the central cornea for the enabling a patient to see immediately after surgery, as in LASIK, without taking too much tissue from the cornea in patients, such as after LASIK in high myopia patients, or those with keratoconus, or hyperopia, etc., which can produce bulging out of the cornea in the postoperative period requiring a full thickness corneal surgery.

At present, elderly patients are not corrected for their presbyopia and astigmatism using excimer laser to ablate their cornea, since they may soon require a lens extraction for their cataract and an appropriate lens implantation and a refractive corneal surgery can make the cornea thin and produce ectasia of the cornea with its corneal complication sometimes requiring a corneal transplantation for its correction, etc. Also, very young children whose eyes grow and require changes in refractive power of one of their eyes are limited in potential treatment methods. In these conditions two types of methodologies can be applied: (i) additive by adding to the corneal stroma a similar compound, such as another corneal stroma that can be obtained from human eye bank or an animal or synthetic from collagen and similar transparent organic materials; or (ii) if the cornea has the sufficient thickness of >500 microns, one can modify the refractive errors to a certain degree, e.g., 20-50 microns. However, in young children with growing eyes and adults above the age of 55 years, the eyes might need a cataract surgery in either case, and the changes of refractive power make the decision for a refractive surgery difficult because it may not be useful for these patients.

Therefore, there is a further need for a refractive surgery that can correct the refractive errors of the eye regardless of the age of the patients or future need for a cataract extraction. Also, a further need exists for a corneal procedure that can be performed simultaneously with any refractive surgery.

Keratoconus is a disease process which is characterized by gradual thinning and out bulging of the cornea leading to distortion of vision, monocular or binocular double vision, nearsightedness, irregular astigmatism, and reduced visual acuity.

A number of factors including genetic predisposition might be involved in the development of keratoconus; since it is found frequently in the siblings, parents or close relatives. However, environmental factors, such as allergies and habitual eye rubbing might also play a role in some patients without known genetic predisposition.

The development of the corneal thinning might be discovered in young adults and myopic eyes which are nearsighted and are progressive with age.

The diagnosis of keratoconus is by examination of the eyes which are often bilateral measuring the corneal curvature by corneal topography. Though the keratoconus can be stabilized in some patients; in most, symptoms are progressive with the age.

Therefore, there is a further need for an apparatus and method for effectively treating keratoconus in patients.

### BRIEF SUMMARY OF EMBODIMENTS OF THE INVENTION

Accordingly, the present invention is directed to a tissue-augmented corneal inlay surgery technique, an ablatable corneal inlay for correction of refractive errors and/or presbyopia, a method of using the ablatable corneal inlay, and a corneal inlay and pinhole lens structure for implantation into a cornea of an eye, that substantially obviates one or more problems resulting from the limitations and deficiencies of the related art.

In accordance with one or more embodiments of the present invention, there is provided a method of correcting refractive errors and presbyopia in an eye of a patient using an ablatable corneal inlay. The method includes the steps of: (i) forming a darkened annular area in a central region of a corneal inlay so as to define a central pinhole for correcting myopia or presbyopia in a host eye of a patient, the darkened annular area being generally non-transparent; (ii) forming a flap or a pocket for receiving the corneal inlay in the cornea of the host eye of the patient; (iii) inserting the corneal inlay into the pocket or on stromal tissue exposed by the flap in the cornea of the host eye of the patient; (iv) modifying the shape of the corneal inlay using a laser so that the corneal inlay is capable of correcting refractive errors in the host eye of the patient; (v) applying a photosensitizer to the cornea of the host eye of the patient so that the photosensitizer permeates at least a portion of the host corneal tissue surrounding the corneal inlay and/or at least a portion of the corneal inlay; and (vi) irradiating the cornea so as to activate cross-linkers in the corneal inlay and/or cross-linkers in the portion of the host corneal tissue surrounding the corneal inlay, and thereby prevent an immune response from the patient, prevent rejection of the corneal inlay by the patient, and/or strengthen the biomechanical properties of the corneal inlay. The corneal inlay is configured to simultaneously correct refractive errors and presbyopia in the host eye of the patient.

In a further embodiment of the present invention, the step of forming the darkened annular area in the central region of the corneal inlay comprises forming the darkened annular area in the central region of the corneal inlay by tattooing using a biocompatible, non-toxic dark or black ink, or a combination of polyvinylidene fluoride and carbon black nanoparticles.

In yet a further embodiment, the step of forming the darkened annular area in the central region of the corneal inlay comprises forming the darkened annular area in the central region of the corneal inlay by means of a darkened polymeric ring that is 3D printed with the corneal inlay.

In still a further embodiment, the step of forming the darkened annular area in the central region of the corneal inlay comprises forming the darkened annular area in the central region of the corneal inlay by inserting a sharp-edged cylinder with darkened outer walls into the corneal inlay or placing a ring made of polyvinylidene fluoride carbon black inside a central hole in the corneal inlay.

In yet a further embodiment, the step of forming the darkened annular area in the central region of the corneal inlay comprises forming the darkened annular area in the central region of the corneal inlay by creating a central aperture in the corneal inlay, and then subsequently tattooing a bounding wall of the central aperture using a biocompatible, non-toxic dark or black ink, or a combination of polyvinylidene fluoride and carbon black nanoparticles; or by subsequently placing a ring made of a biocompatible, non-toxic, dark material inside the central aperture in the corneal inlay so as to form a bounding wall of the central aperture, the biocompatible, non-toxic, dark material forming the bounding wall of the ring comprising a biocompatible, non-toxic dark or black ink, a combination of polyvinylidene fluoride and carbon black nanoparticles, or another non-toxic polymer and carbon black nanoparticles.

In still a further embodiment, the step of forming the darkened annular area in the central region of the corneal inlay comprises forming the darkened annular area in the central region of the corneal inlay by creating a virtual pinhole in the corneal inlay by tattooing using a biocompatible, non-toxic dark or black ink or by 3D printing the virtual pinhole with the darkened annular area made of a combination of polyvinylidene fluoride and carbon black nanoparticles.

In yet a further embodiment, the corneal inlay is formed from a collagen solution using a mold or a 3-D printer, the mold or the 3-D printer being configured to form the corneal inlay into a predetermined shape for correcting a particular refractive error of the patient.

In still a further embodiment, the corneal inlay is formed using the 3-D printer, the 3-D printer including a nozzle for forming the corneal inlay in layers from a collagen solution, and the 3-D printer being under the control of a data processing device so as to form the corneal inlay into a predetermined shape for correcting a particular refractive error of the patient.

In yet a further embodiment, the step of applying the photosensitizer into the cavity of the eye of the patient further comprises applying riboflavin to the cornea of the eye of the patient so that the riboflavin permeates at least a portion of the host corneal tissue surrounding the corneal inlay and/or at least a portion of the corneal inlay; and the step of irradiating the cornea so as to activate cross-linkers in the corneal inlay and/or cross-linkers in the portion of the tissue surrounding the cavity further comprises irradiating the cornea with ultraviolet radiation so as to activate cross-linkers in the corneal inlay and/or cross-linkers in the portion of the host corneal tissue surrounding the corneal inlay.

In still a further embodiment, the step of modifying the shape of the corneal inlay using the laser comprises ablating the corneal inlay using an excimer laser or a femtosecond laser under the control of a Shack-Hartmann wavefront system and a data processing device so as to modify the corneal inlay and a central area of the host corneal tissue to the desired refractive power so that the corneal inlay corrects refractive error of the eye as desired for hyperopia, myopia, astigmatism, or presbyopia after its implantation.

In yet a further embodiment, the corneal inlay is formed from an animal cornea.

In still a further embodiment, the corneal inlay is decellularized using chemical means or gamma radiation, the chemical means for destroying the cellular elements in the corneal inlay are selected from the group consisting of ethanol, glycerol, acids, alkalis, peracetic acid, ammonium hydroxide ionic detergents, sodium dodecyl sulfate, sodium deoxycholate non-ionic detergents, zwitterionic detergents, Triton X-100, benzalkonium chloride, Igepal, genipin, and combinations thereof.

In yet a further embodiment, the corneal inlay is formed from a human cornea.

In still a further embodiment, the step of forming a flap or a pocket for receiving the corneal inlay in the cornea of the host eye of the patient comprises forming the flap in the cornea of the host eye of the patient for receiving the corneal inlay.

In yet a further embodiment, the step of forming a flap or a pocket for receiving the corneal inlay in the cornea of the host eye of the patient comprises forming the pocket in the cornea of the host eye of the patient for receiving the corneal inlay.

In still a further embodiment, a peripheral part of the corneal inlay is formed from a transparent synthetic material, and the step of forming the darkened annular area in the central region of the corneal inlay comprises forming the darkened annular area in the central region of the corneal inlay from a darkened synthetic annular ring attached to the transparent synthetic material forming the peripheral part of the corneal inlay.

In yet a further embodiment, the corneal inlay is in a form of a synthetic organic corneal inlay made of collagen or a combination with other organic materials.

In still a further embodiment, the step of forming the darkened annular area in the central region of the corneal inlay comprises forming the darkened annular area in the central region of the synthetic organic corneal inlay by molding or 3D printing a darkened synthetic annular ring.

In yet a further embodiment, the corneal inlay is formed from a synthetic organic collagen or a combination with other synthetic organic materials using a mold or a 3-D printer, the mold or the 3-D printer being configured to form the corneal inlay into a predetermined shape for correcting a particular refractive error of the patient.

In accordance with one or more other embodiments of the present invention, there is provided a method of corneal implantation with cross-linking. The method comprising the steps of: (i) implanting a corneal inlay into a recipient cornea of an eye of a patient so as to overlie stromal tissue of the recipient cornea; (ii) applying laser energy to a central portion of the corneal inlay and a portion of the stromal tissue of the recipient cornea underneath the corneal inlay so as to modify the refractive power of the eye; (iii) applying a cross-linking solution that includes a photosensitizer to the recipient cornea of the eye of the patient; and (iv) irradiating the corneal inlay and surrounding corneal tissue so as to activate cross-linkers in the corneal inlay and the surrounding corneal tissue, and thereby cross-link the corneal inlay and the surrounding corneal tissue to prevent an immune response to the corneal inlay and/or rejection of the corneal inlay by the patient. In these one or more embodiments, the central portion of the corneal inlay remains clear for the patient without being obstructed by swollen tissue so that the patient is able to see immediately after the corneal inlay surgery.

In a further embodiment of the present invention, prior to implantation of the corneal inlay, the method further comprises the step of: (v) decellularizing and/or damaging the RNA or DNA of the corneal inlay using chemical means, the chemical means for destroying the cellular elements in the corneal inlay are selected from the group consisting of ethanol, glycerol, acids, alkalis, peracetic acid, ammonium hydroxide ionic detergents, sodium dodecyl sulfate, sodium deoxycholate non-ionic detergents, zwitterionic detergents, Triton X-100, benzalkonium chloride, Igepal, genipin, methylene blue, peptide nucleic acids (PNAs), and combinations thereof.

In yet a further embodiment, prior to implantation of the corneal inlay, the method further comprises the steps of: (v) forming a flap in the recipient cornea of the eye so as to expose the stromal tissue of the recipient cornea underlying the flap; (vi) pivoting the flap so as to expose the stromal tissue of the recipient cornea underlying the flap; (vii) implanting the corneal inlay into the recipient cornea of the eye of the patient by inserting the corneal inlay under the flap so as to overlie the exposed stromal tissue of the recipient cornea; and (viii) after applying the laser energy to the central portion of the corneal inlay and the portion of the stromal tissue of the recipient cornea underneath the corneal inlay, covering the corneal inlay with the flap, the corneal inlay being surrounded entirely by the stromal tissue of the recipient cornea.

In still a further embodiment, the step of forming the flap in the recipient cornea of the eye includes cutting the flap using one of: (i) a femtosecond laser and (ii) a mechanical keratome.

In yet a further embodiment, prior to implantation of the corneal inlay, the method further comprises the steps of: (v) forming a pocket in the recipient cornea of the eye of the patient, the pocket being bounded entirely by stromal tissue of the recipient cornea; and (vi) forming a small side incision in the recipient cornea of the eye of the patient, the pocket being accessible through the small side incision in the recipient cornea. In this further embodiment, the step of implanting the corneal inlay into the recipient cornea of the eye of the patient further comprises implanting a preshaped or non-preshaped corneal inlay using an injector into the pocket of the recipient cornea through the small side incision along with a solution containing hyaluronic acid, a low molecular weight heparin, and/or a viscoelastic solution.

In still a further embodiment, the corneal inlay comprises a central pinhole for correcting presbyopia in the eye of the patient, the central pinhole in the corneal inlay being surrounded by a darkened bounding wall.

In yet a further embodiment, the photosensitizer of the cross-linking solution comprises nanoparticles of riboflavin, and wherein the step of irradiating the corneal inlay comprises irradiating the corneal inlay with ultraviolet light.

In still a further embodiment, the laser energy is applied to the corneal inlay using a femtosecond laser and/or an excimer laser so as to modify the refractive power of the corneal inlay for correction of myopia, hyperopia, presbyopia, and/or astigmatism.

In yet a further embodiment, the method further comprises the steps of: (v) after the corneal inlay surgery, applying a medication to the recipient cornea, the medication being selected from the group consisting of a Rock inhibitor, a Wnt inhibitor, an integrin inhibitor, a GSK inhibitor, allopregnanolone, an anti-VEGF, an antibiotic, an anti-viral medication, an anti-fungal medication, a macrolide, and combinations thereof.

In still a further embodiment, the corneal inlay is formed from an animal cornea.

In yet a further embodiment, the corneal inlay is formed from a human eye bank cornea.

In still a further embodiment, the step of applying the laser energy further comprises ablating the corneal inlay using an excimer laser or a femtosecond laser under the control of a Shack-Hartmann wavefront system and a data processing device so as to modify the corneal inlay to the desired refractive power so that the corneal inlay corrects refractive error of the eye as desired for hyperopia, myopia, astigmatism, or presbyopia after its implantation.

In yet a further embodiment, prior to implantation of the corneal inlay, the method further comprises the step of: (v) cutting and/or shaping the corneal inlay to a desired diameter and/or thickness using a trephine, a femtosecond laser, or an excimer laser so as to modify a refractive power of the corneal inlay and form a central pinhole.

In still a further embodiment, the recipient cornea of the eye of the patient is a human cornea.

In yet a further embodiment, the recipient cornea of the eye of the patient is an animal cornea.

In still a further embodiment, the corneal inlay is a molded corneal inlay or a 3-D printed corneal inlay. In this embodiment, the molded corneal inlay or the 3-D printed corneal inlay may be formed from synthetic collagen or another suitable biocompatible synthetic material.

In yet a further embodiment, the method further comprises the steps of: (v) cutting a peripheral boundary around a three-dimensional interior section of the recipient cornea of the eye; (vi) forming a small side incision in the recipient cornea of the eye of the patient so as to gain access to the three-dimensional interior section of the recipient cornea; and (vii) removing the three-dimensional interior section of the recipient cornea from the eye so as to form a pocket with the anterior portion of the recipient cornea remaining intact over the pocket.

In still a further embodiment, a femtosecond laser is used to cut the peripheral boundary around the three-dimensional interior section of the recipient cornea of the eye, and to form the small side incision in the recipient cornea of the eye; and a pair of forceps is used to remove the three-dimensional interior section of the recipient cornea from the eye so as to form the pocket.

In yet a further embodiment, the step of applying laser energy to the central portion of the corneal inlay and the portion of the stromal tissue of the recipient cornea underneath the corneal inlay further comprises forming a cavity through the entire thickness of an area of the corneal inlay and into a top surface section of the stromal tissue.

In still a further embodiment, the cavity formed through the entire thickness of the area of the corneal inlay and into the top surface section of the stromal tissue has a concave shape.

In yet a further embodiment, the corneal inlay comprises a central pinhole, the central pinhole in the corneal inlay being surrounded by a darkened bounding wall; and the step of applying laser energy to the central portion of the corneal inlay and the portion of the stromal tissue of the recipient cornea underneath the corneal inlay further comprises forming a convex central portion of the corneal inlay that includes the central pinhole, the convex central portion of the corneal inlay having a sloped wall surrounding the central pinhole, the central pinhole and the sloped wall of the convex central portion configured to correct hyperopia in the eye of the patient.

In accordance with yet one or more other embodiments of the present invention, there is provided a corneal inlay for implantation into a cornea of an eye. The corneal inlay includes an inlay body formed from natural tissue of a donor cornea; and a central pinhole disposed in the inlay body, the central pinhole being surrounded by a darkened bounding wall.

In a further embodiment of the present invention, the darkened bounding wall in the inlay body is formed by initially tattooing a central area of the inlay body using a biocompatible, non-toxic dark or black ink, and then subsequently cutting the central pinhole in the central area of the inlay body using a trephine or a femtosecond laser.

In yet a further embodiment, the tattooed central area of the inlay body is crosslinked so as to kill pathogenic organisms around the central pinhole and extend a length of time that the biocompatible, non-toxic dark or black ink remains embedded in the inlay body.

In still a further embodiment, the darkened bounding wall is formed by inserting a darkened ring member into an aperture formed in a central area of the inlay body.

In yet a further embodiment, the darkened ring member is formed from polyvinylidene fluoride carbon black mixed with an acrylic polymer.

In still a further embodiment, the central pinhole of the inlay body is a circular-shaped pinhole, an oval-shaped pinhole, or a slit-shaped pinhole.

In yet a further embodiment, a darkening compound used to form the darkened bounding wall in the inlay body comprises carbon black nanoparticles.

In accordance with still one or more other embodiments of the present invention, there is provided a pinhole lens structure that includes an insertable pinhole body defining a through pinhole of approximately 0.9-2.0 mm in diameter with a surrounding darkened wall having an approximately 0.1-1.0 mm wall thickness, the insertable pinhole body configured to be inserted into a corneal pocket in a cornea of an eye.

In a further embodiment of the present invention, the corneal pocket has a diameter of approximately 2.0-3.0 mm.

In yet a further embodiment, the insertable pinhole body is formed from polyvinylidene fluoride carbon black mixed with an acrylic polymer.

It is to be understood that the foregoing general description and the following detailed description of the present invention are merely exemplary and explanatory in nature. As such, the foregoing general description and the following detailed description of the invention should not be construed to limit the scope of the appended claims in any sense.

### BRIEF DESCRIPTION OF THE SEVERAL VIEWS OF THE DRAWINGS

The invention will now be described, by way of example, with reference to the accompanying drawings, in which:

FIG. 1A is a partial side cross-sectional view of an eye having a scarred cornea, wherein substantially the entire thickness of the cornea is scarred;

FIG. 1B is a partial side cross-sectional view of a donor cornea undergoing cross-linking;

FIG. 1C is a partial side cross-sectional view of the eye of FIG. 1A, wherein the scarred cornea is shown being removed;

FIG. 1D is a partial side cross-sectional view of the eye of FIG. 1A, wherein the cross-linked donor cornea is shown being implanted in the location previously occupied by the scarred cornea;

FIG. 2A is a partial side cross-sectional view of an eye having internal corneal scar tissue;

FIG. 2B is a partial side cross-sectional view of the eye of FIG. 2A, wherein the scarred corneal tissue has been externally removed from the eye;

FIG. 2C is a partial side cross-sectional view of the eye of FIG. 2A, wherein a cross-linked donor cornea is shown being implanted in the location previously occupied by the scarred corneal tissue;

FIG. 3A is a partial side cross-sectional view of an eye having internal corneal scar tissue;

FIG. 3B is a partial side cross-sectional view of the eye of FIG. 3A, wherein the scarred corneal tissue is shown being internally removed from the eye;

FIG. 3C is a partial side cross-sectional view of the eye of FIG. 3A, wherein a cross-linked donor cornea is shown being implanted in the location previously occupied by the scarred corneal tissue;

FIG. 4A is a partial side cross-sectional view of an eye having a T-shaped corneal scar and/or diseased tissue portion;

FIG. 4B is another partial side cross-sectional view of a donor cornea undergoing cross-linking;

FIG. 4C is a partial side cross-sectional view illustrating a T-shaped portion of the cross-linked donor cornea being cut out from a remainder of the donor cornea;

FIG. 4D is a partial side cross-sectional view of the eye of FIG. 4A, wherein the T-shaped scarred and/or diseased portion of corneal tissue has been removed from the eye;

FIG. 4E is a partial side cross-sectional view of the eye of FIG. 4A, wherein the cross-linked T-shaped donor cornea portion is shown being implanted in the location previously occupied by the scarred and/or diseased corneal tissue portion;

FIG. 5A illustrates an alternative configuration for the cross-linked donor cornea implant, wherein the donor cornea implant has a dumbbell shape;

FIG. 5B illustrates another alternative configuration for the cross-linked donor cornea implant, wherein the donor cornea implant has a reversed or upside down T-shape;

FIG. 6A is a side cross-sectional view of a host eye prior to an transplant procedure;

FIG. 6B is another side cross-sectional view of the host eye of FIG. 6A, which illustrates a creation of a corneal pocket therein;

FIG. 6C is another side cross-sectional view of the host eye of FIG. 6A, which illustrates an implantation of the cross-linked lamellar lenslet into the host eye;

FIG. 7A is a partial side cross-sectional view of a donor cornea being cross-linked prior to being shaped for use in a transplant procedure;

FIG. 7B is another partial side cross-sectional view of the donor cornea of FIG. 7A, which illustrates the cutting of a cross-linked lamellar lenslet from a remainder of the cross-lined donor cornea;

FIG. 7C is a side cross-sectional view of the cross-linked lamellar lenslet after it has been appropriately shaped and removed from the donor cornea of FIGS. 7A and 7B;

FIG. 8 is a partial side cross-sectional view illustrating the formation of a two-dimensional cut into a cornea of an eye, according to another embodiment of the invention;

FIG. 9 is another partial side cross-sectional view of the eye of FIG. 8, which illustrates the creation of a three-dimensional pocket in the cornea of the eye;

FIG. 10 is yet another partial side cross-sectional view of the eye of FIG. 8, which illustrates the injection of a photosensitizer into the three-dimensional pocket in the cornea of the eye;

FIG. 11A is still another partial side cross-sectional view of the eye of FIG. 8, which illustrates the irradiation of the stromal tissue surrounding the three-dimensional pocket of the eye using ultraviolet radiation delivered from outside of the cornea;

FIG. 11B is yet another partial side cross-sectional view of the eye of FIG. 8, which illustrates the irradiation of the stromal tissue surrounding the three-dimensional pocket of the eye using a fiber optic delivering ultraviolet radiation inside the three-dimensional pocket, according to an alternative embodiment of the invention;

FIG. 12 is still another partial side cross-sectional view of the eye of FIG. 8, which illustrates a lens implant inserted into the pocket so as to change the refractive properties of the eye;

FIG. 13 is yet another partial side cross-sectional view of the eye of FIG. 8, which illustrates the reinjection of a photosensitizer into the three-dimensional pocket with the lens implant disposed therein so that the cross-linking procedure may be repeated;

FIG. 14 is still another partial side cross-sectional view of the eye of FIG. 8, which illustrates the re-irradiation of the stromal tissue surrounding the three-dimensional pocket of the eye during the repetition of the cross-linking procedure;

FIG. 15 is a side cross-sectional view illustrating the creation of a lens implant from an organic block of polymer using a excimer laser;

FIG. 16 is a side cross-sectional view illustrating the cutting of a lens implant from an organic block of polymer using a femtosecond laser;

FIG. 17 is a side cross-sectional view illustrating a lens implant that has been formed using a three-dimensional printing technique or a molding technique;

FIG. 18 is a front view of a cornea of an eye, according to yet another embodiment of the invention;

FIG. 19 is another front view of the cornea of the eye of FIG. 18, wherein a square-shaped intrastromal pocket has been formed in the cornea of the eye;

FIG. 20 is yet another front view of the cornea of the eye of FIG. 18, wherein a circular three-dimensional portion of tissue having a first diameter has been removed from the area within the square-shaped intrastromal pocket;

FIG. 21 is still another front view of the cornea of the eye of FIG. 18, wherein a circular three-dimensional portion of tissue having second diameter has been removed from the area within the square-shaped intrastromal pocket, the second diameter of the circular three-dimensional portion of tissue in FIG. 21 being larger than the first diameter of the circular three-dimensional portion of tissue in FIG. 20;

FIG. 22 is yet another front view of the cornea of the eye of FIG. 18, wherein a circular lens implant has been implanted in the area where the circular three-dimensional portion of tissue has been removed, and wherein a photosensitizer is being injected into the pocket in the cornea of the eye;

FIG. 23 is still another front view of the cornea of the eye of FIG. 18, wherein the circular lens implant is shown in the area where the circular three-dimensional portion of tissue was removed;

FIG. 24A is a partial side cross-sectional view illustrating the forming of a pocket in an eye, according to an embodiment of the invention;

FIG. 24B is a front view of the eye of FIG. 24A, which illustrates the forming of the pocket in the eye;

FIG. 25A is another partial side cross-sectional view of the eye of FIG. 24A, which illustrates the irradiation of the stromal tissue surrounding the pocket of the eye;

FIG. 26A is yet another partial side cross-sectional view of the eye of FIG. 24A, which illustrates the insertion of a lens implant into the pocket so as to change the refractive properties of the eye;

FIG. 26B is a front view of the eye of FIG. 24A, which illustrates the insertion of the lens implant into the pocket of the eye;

FIG. 27A is still another partial side cross-sectional view of the eye of FIG. 24A, which illustrates the application of laser energy to the lens implant in the pocket so as to correct refractive errors of the lens implant and/or the eye in a non-invasive manner;

FIG. 27B is a front view of the eye of FIG. 24A, which illustrates the lens implant in the eye after the refractive power of the lens implant has been modified by the application of the laser energy;

FIG. 28 is a side view of a mold being used to form a synthetic concave lenslet;

FIG. 29 is a side view of a mold being used to form a synthetic convex lenslet;

FIG. 30 is a top view of a circular lenslet;

FIG. 31 is a top view of a circular lenslet optic with a rectangular peripheral edge;

FIG. 32 is a side view of a mold with a vertical lip that is separable from the base;

FIG. 33A is a side view of a mold with a vertical lip that is joined with the base;

FIG. 33B is a side view of a mold with a plunger for compressing the collagen hydrogel disposed in the mold;

FIG. 34A is a side view of a mold being used to form a synthetic lenslet with parallel front and back surfaces;

FIG. 34B is a side view of the synthetic lenslet with parallel front and back surfaces formed by the mold;

FIG. 34C is a side view of the synthetic lenslet being ablated using an excimer so as to form a convex lenslet;

FIG. 34D is a side view of the synthetic lenslet being ablated using an excimer laser so as to form a concave lenslet;

FIG. 35A is a side view of a synthetic lenslet being cut using a femtosecond laser so as to form a concave lenslet;

FIG. 35B is a side view of the synthetic concave lenslet after being cut using the femtosecond laser;

FIG. 36A is a side view of a synthetic lenslet being cut using a femtosecond laser so as to form a convex lenslet;

FIG. 36B is a side view of the synthetic convex lenslet after being cut using the femtosecond laser;

FIG. 37A is a front view of a corneal inlay, according to still another embodiment of the invention;

FIG. 37B is another front view of the corneal inlay after the central region has been darkened so as to be nontransparent;

FIG. 37C is yet another front view of the corneal inlay after a pinhole has been formed in the darkened central region;

FIG. 37D is still another front view of the corneal inlay, which depicts the crosslinking of the corneal inlay using ultraviolet (UV) radiation;

FIG. 37E is a side cross-sectional view of the corneal inlay of FIG. 37C;

FIG. 38A is a front view of a corneal inlay, according to yet another embodiment of the invention, wherein a darken flat ring is provided in the corneal inlay;

FIG. 38B is a side cross-sectional view of the corneal inlay of FIG. 38A;

FIG. 38C is a front view of the corneal inlay of FIG. 38A with transparent peripheral portion of the corneal inlay removed;

FIG. 39A is a side cross-sectional view of a cornea of an eye;

FIG. 39B is another side cross-sectional view of the cornea of FIG. 39A illustrating the formation of a corneal flap and an insertion of the corneal inlay underneath the flap;

FIG. 39C is another side cross-sectional view of the cornea and corneal inlay of FIG. 39B, wherein an excimer laser is being used to ablate the corneal inlay;

FIG. 39C is a side cross-sectional view of the cornea and corneal inlay of FIG. 39B after the flap has been closed, and the cornea and corneal inlay are being cross-linked using ultraviolet (UV) radiation;

FIG. 40A is a side cross-sectional view of a cornea of an eye, which illustrates a creation of a corneal pocket therein;

FIG. 40B is another side cross-sectional view of the cornea of FIG. 40A illustrating a corneal inlay inserted in the pocket;

FIG. 40C is another side cross-sectional view of the cornea and corneal inlay of FIG. 40B, wherein an excimer laser is being used to ablate the corneal inlay in the pocket as part of a PRK procedure;

FIG. 40D is yet another side cross-sectional view of the cornea and corneal inlay of FIG. 40B, wherein the cornea and corneal inlay are being cross-linked using ultraviolet (UV) radiation;

FIG. 41A is a perspective view of a cylinder with darkened outer walls for forming a pinhole in a corneal inlay;

FIG. 41B is a side cross-sectional view depicting the cylinder with darkened outer walls inserted into a corneal implant;

FIG. 41C is a side cross-sectional view depicting a corneal implant with a pinhole having darkened walls formed therein;

FIG. 41D is a side cross-sectional view of an eye with a corneal implant disposed in the cornea of the eye;

FIG. 42A is a front view of a corneal inlay, according to yet another embodiment of the invention;

FIG. 42B is another front view of the corneal inlay after a virtual pinhole has been formed in the corneal inlay;

FIG. 42C is a side cross-sectional view of the corneal inlay of FIG. 42A;

FIG. 42D is a side cross-sectional view of the corneal inlay of FIG. 42B with the virtual pinhole formed in the corneal inlay;

FIG. 43 is a cross-sectional view of an eye illustrating a formation of a corneal flap in the eye using a femtosecond laser;

FIG. 44A is another cross-sectional view of the eye of FIG. 43, wherein the corneal flap is shown being rotated to expose stromal tissue of the cornea underneath the flap;

FIG. 44B is a top plan view of the eye of FIG. 44A, wherein the corneal flap is shown rotated to expose the stromal tissue of the cornea underneath the flap;

FIG. 45A is yet another cross-sectional view of the eye of FIG. 43, wherein a corneal inlay has been placed on the exposed stromal tissue of the cornea, and an excimer laser is shown ablating the corneal inlay and a central portion of the host corneal stroma;

FIG. 45B is a top plan view of the eye of FIG. 45A, wherein an excimer laser is shown ablating the corneal inlay and the central portion of the host corneal stroma;

FIG. 46A is still another cross-sectional view of the eye of FIG. 43, wherein the corneal inlay and the central portion of the host corneal stroma has been ablated by the excimer laser under the control of a data processing device with software executed thereby;

FIG. 46B is a top plan view of the eye of FIG. 46A, wherein the corneal inlay and the central portion of the corneal stroma has been ablated by the excimer laser;

FIG. 47A is yet another cross-sectional view of the eye of FIG. 43, wherein the corneal flap is shown replaced back onto the cornea overlapping the corneal inlay;

FIG. 47B is a top plan view of the eye of FIG. 47A, wherein the corneal inlay and the surrounding corneal tissue is shown being irradiated with ultraviolet light, after administration of riboflavin, so as to crosslink and kill all pathogens in the corneal inlay and the surrounding corneal tissue, and strengthen the biomechanics of the cornea and correct the refractive errors;

FIG. 48A is a cross-sectional view of an eye illustrating a corneal pocket in an eye formed using a femtosecond laser;

FIG. 48B is another cross-sectional view of the eye of FIG. 48A, wherein a doughnut-shaped corneal inlay has been implanted in the corneal pocket;

FIG. 48C is a top plan view of the doughnut-shaped corneal inlay of FIG. 48B with a 1-3 mm diameter hole;

FIG. 49 is a top plan view of an eye illustrating a corneal flap that has been rotated to expose stromal tissue of the cornea underneath the flap;

FIG. 50A is another top plan view of the eye of FIG. 49, wherein a corneal inlay has been placed on the exposed stromal tissue of the cornea;

FIG. 50B is yet another top plan view of the eye of FIG. 49, wherein a central hole has been formed in the corneal inlay;

FIG. 51 is still another top plan view of the eye of FIG. 49, wherein an excimer laser is shown ablating the corneal inlay to correct the refractive error of the eye;

FIG. 52 is yet another top plan view of the eye of FIG. 49, wherein a central hole has been formed in the corneal inlay using a trephine;

FIG. 53 is still another top plan view of the eye of FIG. 49, wherein a pinhole ring has been inserted in the corneal inlay that has been irradiated with an excimer laser;

FIG. 54 is a top plan view of an eye illustrating a corneal inlay and a pinhole ring that has been inserted into a corneal pocket through an incision in the cornea;

FIG. 55 is a top plan view illustrating a cornea of an eye;

FIG. 56 is another top plan view of the eye of FIG. 55, wherein a corneal pocket has been formed in a central portion of the cornea, and the corneal pocket is accessible by means of a side incision and intrastromal channel in the cornea;

FIG. 57 is yet another top plan view of the eye of FIG. 55, wherein a pinhole ring has been inserted into the corneal pocket by means of the side incision and intrastromal channel in the cornea;

FIG. 58 is a partial side cross-sectional view of an eye with a normal cornea;

FIG. 59 is a partial side cross-sectional view of an eye with a thinned cornea due to keratoconus;

FIG. 60 is a partial side cross-sectional view of an eye with a scleral lens disposed over the cornea of the eye, according to an embodiment of the invention;

FIG. 61 is a cross-sectional view of a scleral lens formed from a soft internal polymer and a rigid external polymer, according to another embodiment of the invention, wherein a flanged synthetic pin or plug having a darkened wall and a clear transparent center is positioned in an opening in the rigid external polymer of the scleral lens;

FIG. 62A is a cross-sectional view of a transitional scleral lens formed from a soft internal polymer and a rigid external polymer, according to yet another embodiment of the invention, wherein the scleral lens is disposed in a dark condition such that the rigid external polymer, which contains light sensitive molecules of the transitional scleral lens, is light in color;

FIG. 62B is another cross-sectional view of the transitional scleral lens of FIG. 62A, wherein the scleral lens is disposed in a bright light condition such that the rigid external polymer, which contains light sensitive molecules of the transitional scleral lens, is dark in color;

FIG. 62C is a cross-sectional view of a scleral lens formed from a soft internal polymer and a rigid external polymer, according to still another embodiment of the invention, wherein a non-flanged synthetic pin or plug having a darkened wall and a clear transparent center is positioned in an opening in the rigid external polymer of the scleral lens;

FIG. 63A is a front view of the scleral lens of FIG. 62C;

FIG. 63B is a front view of a scleral lens with a central darkened wall pinhole formed from a double stained polymer, according to yet another embodiment of the invention;

FIG. 63C is a cross-sectional view of the scleral lens of FIG. 63B, wherein the central darkened wall pinhole formed from the double stained polymer has a transparent center bounded by dark walls;

FIG. 63D is a cross-sectional view of a scleral lens similar to that of FIG. 63C, except that the scleral lens is provided with a soft internal polymer in addition to the rigid external polymer;

FIG. 64 is a top plan view illustrating a cornea of an eye;

FIG. 65 is a side cross-sectional view of a corneal inlay with a constant thickness;

FIG. 66 is a side cross-sectional view of a concave corneal inlay, wherein the concave corneal inlay has been formed by molding;

FIG. 67 is a side cross-sectional view of a convex corneal inlay, wherein the convex corneal inlay has been formed by molding;

FIG. 68 is another top plan view of the cornea of the eye of FIG. 64, wherein a corneal flap, which has been formed in the eye, is shown rotated to expose stromal tissue of the cornea underneath the flap;

FIG. 69 is still another top plan view of the cornea of the eye of FIG. 64, wherein the concave corneal inlay of FIG. 66 has been placed on the stromal tissue disposed underneath the corneal flap;

FIG. 70 is yet another top plan view of the cornea of the eye of FIG. 64, wherein the convex corneal inlay of FIG. 67 has been placed on the stromal tissue disposed underneath the corneal flap;

FIG. 71 is a top plan view illustrating a cornea of an eye with a corneal pocket formed in the cornea, wherein a corneal inlay has been inserted into the corneal pocket;

FIG. 72 is another side cross-sectional view of the corneal inlay of FIG. 65, wherein a laser is being used to ablate the corneal inlay;

FIG. 73 is a side cross-sectional view of a concave corneal inlay that has been formed from the corneal inlay of FIG. 72 by ablation, and a central pinhole surrounded by a darkened wall has been formed in the corneal inlay;

FIG. 74 is a side cross-sectional view of a convex corneal inlay that has been formed from the corneal inlay of FIG. 72 by ablation, and a central pinhole surrounded by a darkened wall has been formed in the corneal inlay;

FIG. 75 is a side cross-sectional view of an eye;

FIG. 76 is another side cross-sectional view of the eye of FIG. 75 illustrating a formation of a corneal flap in the eye using a femtosecond laser;

FIG. 77 is yet another side cross-sectional view of the eye of FIG. 75 illustrating an ablation of corneal tissue underneath the corneal flap after the corneal flap has been rotated to expose the corneal tissue;

FIG. 78 is yet another side cross-sectional view of the eye of FIG. 75, wherein the corneal flap is shown replaced back onto the cornea over the ablated corneal tissue;

FIG. 79 is a side cross-sectional view of an eye illustrating a formation of a corneal flap in the eye using a femtosecond laser;

FIG. 80 is another side cross-sectional view of the eye of FIG. 79, wherein a corneal inlay has been placed on exposed corneal tissue after the corneal flap has been rotated to expose the corneal tissue;

FIG. 81A is yet another side cross-sectional view of the eye of FIG. 79, wherein an excimer laser is being used to ablate the corneal inlay;

FIG. 81B is an enlarged detail view of a portion of the inlay and cornea of FIG. 81A to illustrate that the ablated concave portion of the inlay does not extend into the corneal tissue beneath the inlay;

FIG. 82A is an enlarged detail view of a portion of an inlay, cornea, and corneal flap of an eye, which illustrates an ablated concave portion of an inlay that extends into corneal tissue beneath the inlay;

FIG. 82B is a side cross-sectional view of the eye of FIG. 82A having the inlay, cornea, and corneal flap disposed over the inlay;

FIG. 83 is a side cross-sectional view of a cornea of an eye, wherein a corneal inlay has been placed on exposed corneal tissue after a corneal flap has been rotated to expose the corneal tissue;

FIG. 84 is another side cross-sectional view of the cornea of the eye of FIG. 83, which illustrates an ablated concave portion of a corneal inlay formed by an excimer laser that extends into corneal tissue beneath the corneal inlay;

FIG. 85 is yet another side cross-sectional view of the cornea of the eye of FIG. 83, wherein the corneal flap is shown replaced back onto the cornea over the ablated concave portion of the corneal inlay that extends into corneal tissue beneath the corneal inlay, and the corneal inlay and the surrounding corneal tissue is undergoing crosslinking;

FIG. 86 is a side cross-sectional view of an eye;

FIG. 87 is another side cross-sectional view of the eye of FIG. 86 illustrating a formation of a corneal pocket in the eye using a spatula knife;

FIG. 88 is a partial side cross-sectional view of the cornea of the eye of FIG. 86, wherein a corneal inlay has been inserted into the corneal pocket;

FIG. 89 is another partial side cross-sectional view of the cornea of the eye of FIG. 86, wherein a corneal inlay with a central pinhole surrounded by a darkened wall has been inserted into the corneal pocket;

FIG. 90 is yet another partial side cross-sectional view of the cornea of the eye of FIG. 86, wherein a corneal inlay has an ablated concave portion formed by a femtosecond laser that extends into a corneal stroma beneath the corneal inlay;

FIG. 91 is a top view of a corneal inlay with a central pinhole and an ablated annular portion surrounding the central pinhole;

FIG. 92 is a side cross-sectional view of the corneal inlay of FIG. 91;

FIG. 93 is a side cross-sectional view of a cornea of an eye, wherein the corneal inlay of FIGS. 91 and 92 with the central pinhole, the darkened wall surrounding the central pinhole, and the ablated concave annular portion surrounding the darkened wall of the central pinhole has been inserted into a corneal pocket;

FIG. 94 is a top view of a corneal inlay formed from natural tissue, wherein the central area of the corneal inlay is tattooed;

FIG. 95 is another top view of the corneal inlay of FIG. 94, wherein a central portion of the corneal inlay has been removed using a trephine or femtosecond laser so as to leave a tattooed ring surrounding a central aperture through which light passes unhindered;

FIG. 96 is a top view of a corneal inlay formed from natural tissue, wherein the corneal inlay comprises a central pinhole aperture formed through a center thereof;

FIG. 97 is another top view of the corneal inlay of FIG. 96, wherein a synthetic darkened ring has been inserted into the central pinhole aperture, thereby providing a central pinhole with a darkened wall inside the corneal inlay;

FIG. 98 is a top view of a cornea of an eye, wherein a corneal pocket has been formed in the center of the eye;

FIG. 99 is another top view of the cornea of FIG. 98, wherein tissue inside the corneal pocket is being ablated using a femtosecond laser so that the tissue can be removed from the eye;

FIG. 100 is another top view of the cornea of FIG. 99, wherein the corneal pocket disposed in the corneal stroma underneath the Bowman's membrane is depicted; and

FIG. 101 is another top view of the cornea of FIG. 99, wherein a synthetic darkened ring with a central pinhole is inserted into the corneal pocket of the cornea through a small side opening in the corneal pocket.

Throughout the figures, the same elements are always denoted using the same reference characters so that, as a general rule, they will only be described once.

### DETAILED DESCRIPTION OF EMBODIMENTS OF THE INVENTION

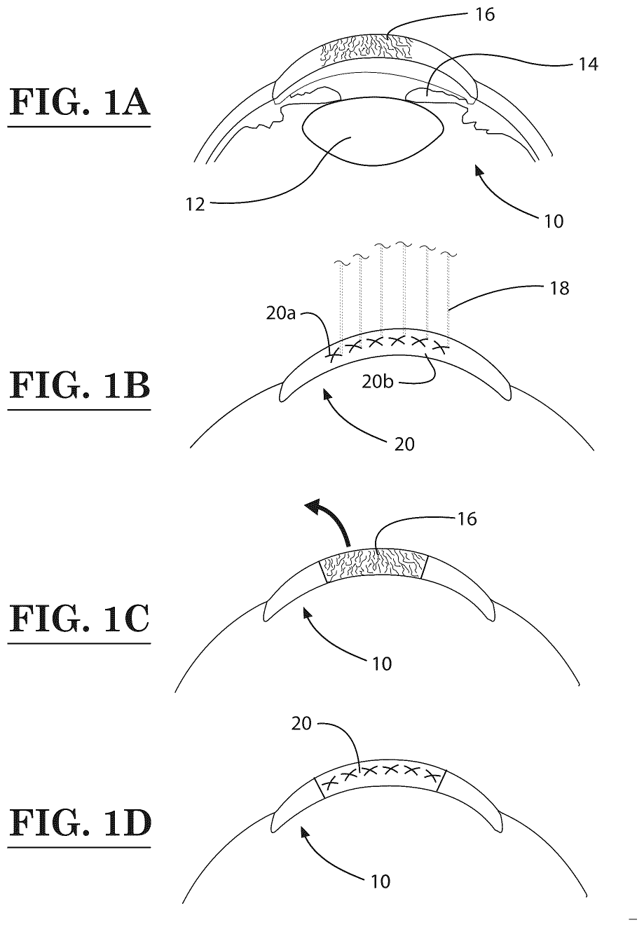

A first illustrative embodiment of a corneal transplant procedure with a cross-linked cornea is shown in FIGS. 1A-1D. The corneal transplant procedure illustrated in FIGS. 1A-1D involves full corneal replacement of the scarred or diseased cornea by the donor cornea. In other words, FIGS. 1A-1D illustrate a penetrating keratoplasty procedure wherein the full thickness of the scarred or diseased cornea is replaced with a cross-linked donor cornea (i.e., a full-thickness corneal transplant).

Referring initially to FIG. 1A, it can be seen that substantially the entire thickness of the cornea 16 of the eye 10 is scarred and/or diseased (i.e., scarred, diseased, or scarred and diseased). FIG. 1A also illustrates the lens 12 and iris 14 of the eye 10, which are located posteriorly of the cornea 16. In this embodiment, it is necessary to replace substantially the entire thickness of the cornea 16 with a donor cornea.

In FIG. 1B, the cross-linking 18 of the clear donor cornea 20 is diagrammatically illustrated. As depicted in FIG. 1B, only the front portion 20a of the donor cornea 20 is cross-linked. That is, the cross-linking does not extend all the way to the rear portion 20b of the donor cornea 20. It is to be understood that the cross-linking 18 of the donor cornea 20 may also be done after implanting the donor cornea into the eye of the patient, rather than before implantation as shown in the illustrative example of FIGS. 1A-1D. Also, it is to be understood that all or just a part of the donor cornea 20 may be cross-linked.

In the illustrative embodiments described herein (i.e., as depicted in FIGS. 1A-1D, 2A-2C, and 3A-3C), the cross-linking of the clear donor cornea may comprise the steps of: (i) applying a photosensitizer to the donor cornea, the photosensitizer facilitating cross-linking of the donor cornea; and (ii) irradiating the donor cornea with ultraviolet light so as to activate cross-linkers in the donor cornea and thereby strengthen the donor cornea. The photosensitizer may comprise riboflavin or a solution comprising a liquid suspension having nanoparticles of riboflavin. The cross-linker may have between about 0.1% Riboflavin to about 100% Riboflavin or any other suitable range or specific percentage therein. The ultraviolet radiation or rays used to irradiate the donor cornea may be between about 370 nanometers and about 380 nanometers (or between 370 nanometers and 380 nanometers). The radiation is preferably about 3 mW or more as needed and emanates from a laser source at about a 3 cm distance from the donor cornea for about 30 minutes or less. The time of the exposure can vary depending on the light intensity, focus, and the concentration of riboflavin. However, the ultraviolet radiation can be applied at any suitable distance, time or wavelength. Preferably, cross-linking the donor cornea does not significantly change the refractive power of the donor cornea; however, if desired, cross-linking can change the refractive power of the donor cornea to any suitable degree.

In addition to Riboflavin, other suitable cross linking agents are low carbon carbohydrates, such as pentose sugar (e.g., ribose) or hexose sugar (e.g., glucose), or complex carbohydrates. Other crosslinking agents may include Transaminidases, transglutaminases or a naturally-derived cross-linker named malic acid derivative (MAD) concentrations higher than 30 mM, commercially available cross-linkers such as 1-ethyl-3-(3 (′-dimethylaminopropyl) carbodiimide (EDC), or ethyl-3 (3-dimethylamino) propyl carbodiimide (EDC), etc. The cross-linking may also be done postoperatively by the application of other crosslinking agents, such as Triglycidylamine (TGA) synthesized via reacting epichlorhydrin and a carbodiimide, or the oxidized glycogen hexoses. The ribose, glucose and similar agents may penetrate the cornea easily using drops, gel, or the slow release mechanisms, nanoparticle, microspares, liposome sets. In addition, the crosslinkers may be delivered with Mucoadhesives.

In one or more embodiments, all or part of the donor cornea is cross-linked. Also, in one or more embodiments, a very high concentration of Riboflavin may be used because the in vitro cross-linking process may be stopped whenever needed prior to the transplantation of the donor cornea in the host eye. In addition, the power of the ultraviolet (UV) laser may also be increased so as to cross-link the tissue of the donor cornea faster. The use of a high concentration of Riboflavin, and the increasing of the ultraviolet (UV) laser power, are not possible during an in vivo cross-linking procedure because the aim of such an in vivo procedure is to protect the cells of the host cornea. Also, the in vivo process cannot be controlled as efficiently as in the vitro crosslinking of the corneal transplant.

In one or more embodiments, the donor cornea may be extracted from a human cadaver, or the cornea may be reconstructed as known in tissue engineering in vitro and three-dimensionally (3D) printed. Cross-linking of a culture-grown cornea eliminates the cellular structure inside the cornea. If needed again, the healthy corneal endothelium of the patient may be grown in vitro for these tissues by placing them on the concave surface of the cornea and encouraging their growth under laboratory control conditions prior to the transplantation.

In the embodiments where the donor cornea is tissue culture grown, the cornea may be formed from mesenchymal fibroblast stem cells, embryonic stem cells, or cells derived from epithelial stem cells extracted from the same patient, or a mixture of these cells. Using known tissue culture techniques, the cells may produce a transparent corneal stroma. This culture-grown corneal stroma will not have a corneal epithelium or a corneal endothelium. Thus, it eliminates the complexity of developing a full thickness cornea in the tissue culture. This stromal transplant may be used as a lamellar or partial thickness replacement of the existing host cornea. This transplant may also be used to augment or add to the thickness of the host cornea. This transparent corneal stroma may be transplanted either prior to, or after being cross-linked using various cross-linking methods.

In one or more embodiments, the cross-linked donor cornea may be sized and precisely cut with a femtosecond laser to the desired shape and curvature to replace the removed host cornea so that the refractive errors of the recipient are also automatically corrected with the cross-linked cornea.

Now, referring to FIG. 1C, it can be seen that the scarred and/or diseased cornea 16 is shown being removed from the eye 10. The scarred and/or diseased cornea 16 may be removed from the eye 10 by using various suitable means, such as mechanical means or cutting using a laser. When mechanical means are used to remove the scarred and/or diseased cornea 16 from the eye 10, the scarred and/or diseased cornea 16 may initially be cut away or dissected from the remainder of the eye 10 using a sharp mechanical instrument (e.g., a surgical micro-knife, a needle, a sharp spatula, a pair of micro-scissors), and then subsequently removed or extracted with a pair of micro-forceps. When laser cutting is used to remove the scarred and/or diseased cornea 16 from the eye 10, the scarred and/or diseased cornea 16 may be cut away using a suitable laser, such as a femtosecond laser. Also, in some embodiments, the mechanical means for cutting and extraction (e.g., the surgical micro-knife and/or pair of micro-scissors) may be used in combination with the laser means (e.g., the femtosecond laser).

In one or more embodiments, the donor cornea may be shaped and cut with the femtosecond laser prior to the cross-linking thereof so as to replace part or all of the recipient cornea which is cut with the femtosecond laser. In these one or more embodiments, the entire donor and host cornea together may be cross-linked with Riboflavin and UV radiation. These procedures may also be performed on a culture-grown transplant cornea.

Then, as shown in FIG. 1D, after the scarred and/or diseased cornea 16 has been removed from the eye 10, the cross-linked donor cornea 20 is implanted into the eye 10 of the patient in the location previously occupied by the scarred and/or diseased cornea 16. After implantation of the cross-linked donor cornea 20, sutures or a suitable adhesive may be utilized to secure the cross-linked donor cornea 20 in place on the eye 10. When sutures are used for holding the donor cornea 20 in place, the sutures may comprise nylon sutures, steel sutures, or another suitable type of non-absorbable suture. When the cornea 16 is subsequently ablated after the implantation of the donor cornea, as will be described hereinafter, additional sutures may be required after ablation.

In one or more embodiments, a biodegradable adhesive is used in a corneal transplantation procedure with the cross-linked donor cornea 20 described above, or with a non-cross-linked corneal transplant. In these one or more embodiments, the biodegradable adhesive obviates the need for a suture in the corneal transplant procedure. Sutures generally distort the surface of the cornea and can produce an optically unacceptable corneal surface. Also, the use of the biodegradable adhesive obviates the need for glues requiring exothermic energy. Glues that use an exothermic effect, such as Fibronectin, need thermal energy to activate their adhesive properties. This thermal energy, such as that delivered by a high-powered laser, produces sufficient heat to coagulate the Fibronectin and the tissue that it contacts. Any thermal effect on the cornea produces: (i) corneal opacity, (ii) tissue contraction, and (iii) distortion of the optical surface of the cornea. The tissue adhesion created by these glues, including Fibronectin or fibrinogen, is flimsy and cannot withstand the intraocular pressure of the eye.

In fact, sutures are superior to these types of adhesives because the wound becomes immediately strong with sutures, thereby supporting the normal intraocular pressure of between 18 and 35 mmHg. In contrast to the use of a suture in which distortion that is caused by suture placement can be managed by cutting and removing the suture, the distortion caused by the coagulated corneal tissue cannot be corrected.

Other glues, such as cyanoacrylate, become immediately solid after coming into contact with the tissue or water. These glues produce a rock-hard polymer, the shape of which cannot be controlled after administration. Also, the surface of the polymer created by these glues is not smooth. Thus, the eyelid will rub on this uneven surface, and the uneven surface scratches the undersurface of the eyelid when the eyelid moves over it. In addition, the cyanoacrylate is not biodegradable or biocompatible. As such, it causes an inflammatory response if applied to the tissue, thereby causing undesirable cell migration and vascularization of the cornea.

Thus, by using a biocompatible and absorbable acrylate or other biodegradable glues that do not need exothermic energy for the process of adhesion (i.e., like fibronectin or fibrinogen), one is able to maintain the integrity of the smooth corneal surface. In one or more embodiments, the biocompatible and biodegradable adhesive may be painted only at the edges of the transplant prior to placing it in the host or diseased cornea. In these embodiments, the biocompatible and biodegradable adhesive only comes into contact with the host tissue at the desired predetermined surface to create a strong adhesion. The adhesion may last a few hours to several months depending on the composition of the molecule chosen and the concentration of the active component.

Other suitable biodegradable adhesives or glues that may be used in conjunction with the transplant include combinations of gallic acid, gallic tannic acid, Chitosan, gelatin, polyphenyl compound, Tannic Acid (N-isopropylacrylamide (PNIPAM), and/or Poly(N-vinylpyrrolidone) with polyethylene glycol (PEG). That is, polyethylene glycol (PEG) may be mixed with any one or plurality of gallic acid, gallic tannic acid, Chitosan, gelatin, polyphenyl compound, Tannic Acid (N-isopropylacrylamide (PNIPAM), and Poly(N-vinylpyrrolidone), so as to form a molecular glue. These adhesives are suitable for the use on the cornea because they create a tight wound that prevents leakage from the corneal wound and maintain the normal intraocular pressure shortly after their application and also do not distort the wound by causing traction on the tissue.

In one or more embodiments, the donor cornea may be temporarily sutured to the host cornea by only a few single sutures to the host cornea. Then, the sutures may be removed immediately after donor cornea is fixed to the host cornea with a suitable adhesive.

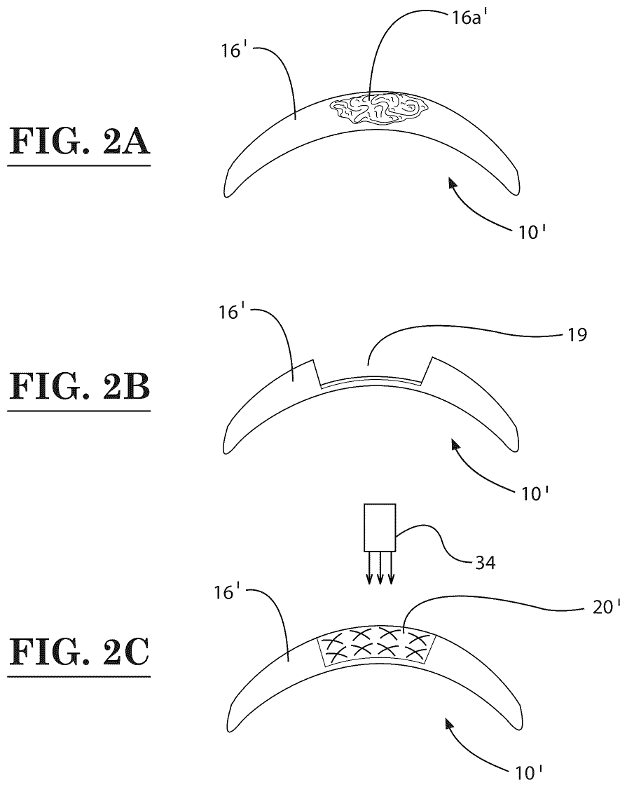

A second illustrative embodiment of a corneal transplant procedure with a cross-linked cornea is shown in FIGS. 2A-2C. Unlike the first embodiment described above, the corneal transplant procedure illustrated in FIGS. 2A-2C does not involve full corneal replacement of the scarred or diseased cornea by the donor cornea. Rather, FIGS. 2A-2C illustrate a lamellar keratoplasty procedure wherein only a portion of the cornea 16′ of the eye 10′ contains scarred and/or diseased tissue (i.e., a full-thickness corneal section is not removed). In the procedure of FIGS. 2A-2C, an internal scarred and/or diseased portion 16a′ of the cornea 16′ is externally removed from the eye 10′ of a patient.

Referring initially to FIG. 2A, it can be seen that only an internal portion 16a′ of the cornea 16′ is scarred and/or diseased. As such, in this embodiment, it is not necessary to replace the entire thickness of the cornea 16 with a donor cornea as was described above in conjunction with FIGS. 1A-1D, but rather just a portion of the cornea 16′.

Next, referring to FIG. 2B, it can be seen that the scarred and/or diseased portion 16a′ has been externally removed from the cornea 16′ of the eye 10′ such that the cornea 16′ comprises a cavity 19 disposed therein for receiving the donor cornea. Because an external approach was utilized for removing the scarred and/or diseased portion 16a′ of the cornea 16′, the cavity 19 comprises a notch-like void in the outside or anterior surface of the cornea 16′. As described above for the first embodiment, the scarred and/or diseased corneal portion 16a′ may be removed from the remainder of the cornea 16′ using various suitable means, such as mechanical means or the laser cutting means (e.g., femtosecond laser) described above.

Finally, as shown in FIG. 2C, after the scarred and/or diseased portion 16a′ has been removed from the remainder of the cornea 16′ of the eye 10′, the cross-linked donor cornea or cross-linked donor corneal portion 20′ is implanted into the eye 10′ of the patient in the location previously occupied by the scarred and/or diseased corneal portion 16a′. As described above, after implantation of the cross-linked donor corneal portion 20′ into the eye 10′, sutures or a suitable adhesive (e.g., the biocompatible and biodegradable adhesive described above) may be utilized to secure the cross-linked donor corneal portion 20′ in place on the host cornea of the eye 10′.

After the cross-linked donor corneal portion 20′ is implanted into the eye 10′ of the patient, a portion of the cornea 16′ may be ablated so as to change the refractive properties of the eye (e.g., to give the patient perfect or near perfect refraction). The ablation of the portion of the cornea 16′ may be performed using a suitable laser 34, such as an excimer laser. The ablation by the laser causes the ablated tissue to essentially evaporate into the air. Also, the ablation of the portion of the cornea 16′ may be done intrastromally, as with LASIK (laser-assisted in situ keratomileusis), or on the surface of the cornea, as with PRK (photorefractive keratectomy). The ablation may be performed a predetermined time period after the corneal transplantation so as to enable the wound healing process of the recipient's cornea to be completed. It is to be understood that the ablation, which follows the corneal transplantation, may be performed in conjunction with any of the embodiments described herein.

It is also to be understood that, in some alternative embodiments, the ablation may be performed prior to the transplantation of the donor cornea, rather than after the transplantation of the donor cornea. For example, in one or more alternative embodiments, a lenticle may be precisely cut in the tissue of a culture-grown stroma of a donor cornea by using a femtosecond laser so that when implanted into the host cornea, it corrects the residual host eye's refractive error.

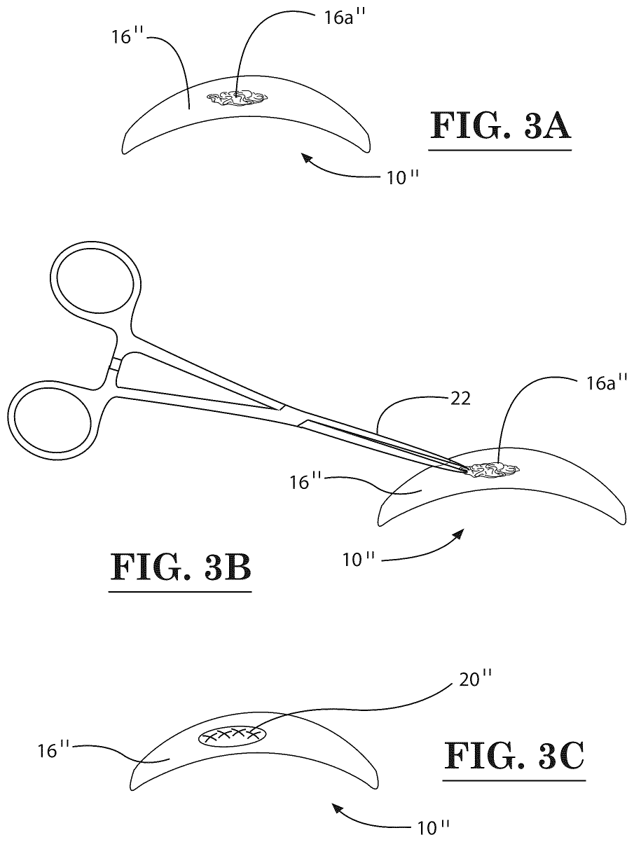

A third illustrative embodiment of a corneal transplant procedure with a cross-linked cornea is shown in FIGS. 3A-3C. Like the second embodiment described above, the corneal transplant procedure illustrated in FIGS. 3A-3C only involves replacing a scarred and/or diseased portion 16a″ of the cornea 16″ with a donor corneal portion. Thus, similar to the second embodiment explained above, FIGS. 3A-3C illustrate a lamellar keratoplasty procedure wherein only a portion of the cornea 16″ of the eye 10″ contains scarred and/or diseased tissue (i.e., a full-thickness corneal section is not removed). Although, in the procedure of FIGS. 3A-3C, an internal scarred and/or diseased portion 16a″ of the cornea 16″ is internally removed from the eye 10″ of a patient, rather than being externally removed as in the second embodiment of FIGS. 2A-2C.

Referring initially to FIG. 3A, it can be seen that only an internal portion 16a″ of the cornea 16″ of the eye 10″ is scarred and/or diseased. As such, in this embodiment, like the preceding second embodiment, it is not necessary to replace the entire thickness of the cornea 16′″ with a donor cornea, but rather just a portion of the cornea 16″.

Next, referring to FIG. 3B, it can be seen that the scarred and/or diseased portion 16a″ is being internally removed from the remainder of the cornea 16″ using a pair of forceps 22 (i.e., mechanical means of removal are illustrated in FIG. 3B). Advantageously, because an internal approach is being utilized for removing the scarred and/or diseased portion 16a″ of the cornea 16″, the cornea 16″ will not comprise the notch-like cavity 19 disposed in the outside or anterior surface of the cornea, which was described in conjunction with the preceding second embodiment. As described above for the first and second embodiments, the scarred and/or diseased corneal portion 16a″ may be removed from the remainder of the cornea 16″ using other suitable alternative means, such as laser cutting techniques (e.g., using a femtosecond laser). Advantageously, the femtosecond laser is capable of cutting inside the tissue without involving the surface of the tissue. The cut part of the tissue can then be removed by other means (e.g., micro-forceps).

Finally, as shown in FIG. 3C, after the scarred and/or diseased corneal portion 16a″ has been removed from the remainder of the cornea 16″ of the eye 10″, the cross-linked donor cornea or cross-linked donor corneal portion 20″ is implanted into the eye 10″ of the patient in the location previously occupied by the scarred and/or diseased corneal portion 16a″. After implantation of the cross-linked donor corneal portion 20″, sutures or a suitable adhesive (e.g., the biocompatible and biodegradable adhesive described above) may be utilized to secure the cross-linked donor corneal portion 20″ in place on the host cornea of the eye 10″. Advantageously, the cross-linked donor corneal portion 20″, which is strengthened by the cross-linking performed thereon, reinforces the cornea 16″ and greatly reduces the likelihood of corneal graft rejection.

It is to be understood that the scarred and/or diseased corneal portion 16a″ that is removed from the cornea 16″ may also be replaced with stroma stem cells or mesenchymal stem cells, which can be contained in a medium, and then injected in the internal cavity previously occupied by the scarred and/or diseased corneal tissue 16a″.

In one or more embodiments, mesenchymal stem cells also may be injected inside the donor cornea before or after transplantation. In addition, in one or more embodiments, daily drops of a Rho Kinase inhibitor may be added to the host eye after the surgery. The use of a medication, such as a Rho Kinase inhibitor, with the stem cells will encourage stem cell proliferation.

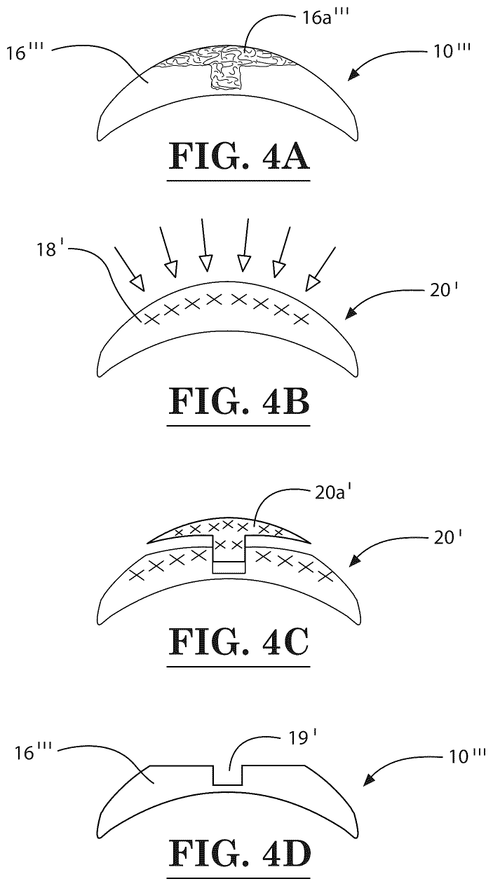

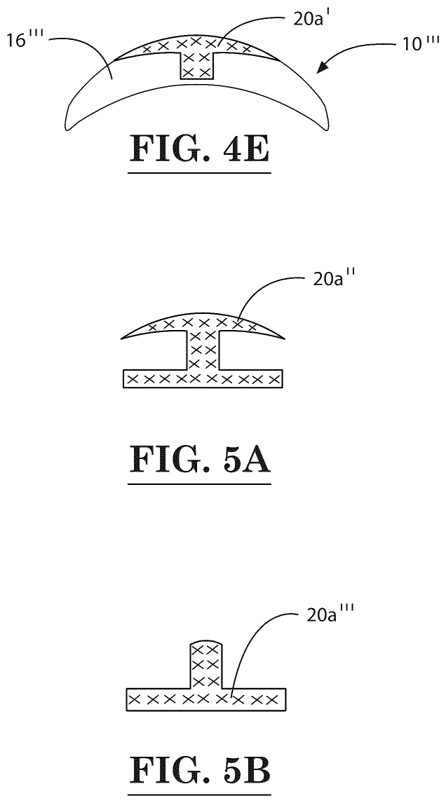

A fourth illustrative embodiment of a corneal transplant procedure with a cross-linked cornea is shown in FIGS. 4A-4E. Like the second and third embodiments described above, the corneal transplant procedure illustrated in FIGS. 4A-4E only involves replacing a scarred and/or diseased portion 16a″″′ of the cornea 16′″ with a donor corneal portion. Thus, similar to the second and third embodiments explained above, FIGS. 4A-4E illustrate a lamellar keratoplasty procedure wherein only a portion of the cornea 16″″′ of the eye 10′″ contains scarred and/or diseased tissue (i.e., a full-thickness corneal section is not removed). Although, in the procedure of FIGS. 4A-4E, a different-shaped scarred and/or diseased portion 16a″″′ of the cornea 16′″″ is removed.

Referring initially to FIG. 4A, it can be seen that only a portion 16a′″ of the cornea 16″″′ having a T-shape or “top hut” shape is scarred and/or diseased. As such, in this embodiment, it is not necessary to replace the entire thickness of the cornea 16″″′ with a donor cornea as was described above in conjunction with FIGS. 1A-1D, but rather just a portion 16a″″ of the cornea 16″″′. In this illustrative embodiment, the back side of the cornea 16′″″ is maintained (see e.g., FIG. 4D).

In FIG. 4B, the cross-linking 18′ of the clear donor cornea 20′ is diagrammatically illustrated. As mentioned above, it is to be understood that all or just a part of the donor cornea 20′ may be cross-linked. Then, in FIG. 4C, it can be seen that a portion 20a′ of the clear donor cornea 20′, which has a T-shape or “top hut” shape that matches the shape of the scarred and/or diseased portion 16a″″ of the cornea 16′″, is cut out from the remainder of the clear donor cornea 20′ such that it has the necessary shape. In one or more embodiments, the portion 20a′ may be cut from the clear donor cornea 20′ and appropriately shaped using a femtosecond laser. As shown in FIGS. 5A and 5B, other suitably shaped cross-linked corneal portions may be cut from the clear donor cornea 20′, such as a dumbbell-shaped corneal portion 20a″ (see FIG. 5A) or a corneal portion 20a′″ having a reversed T-shape or “reversed top hut” shape (see FIG. 5B), in order to accommodate correspondingly shaped scarred and/or diseased areas in the host cornea.

Next, referring to FIG. 4D, it can be seen that the scarred and/or diseased portion 16a″″ having the T-shape or “top hut” shape has been removed from the cornea 16″″′ of the eye 10” such that the cornea 16′″ comprises a cavity 19′ disposed therein for receiving the donor cornea. As described above for the first three embodiments, the scarred and/or diseased corneal portion 16a″″′ may be removed from the remainder of the cornea 16′″″ using various suitable means, such as mechanical means or the laser cutting means (e.g., femtosecond laser) described above.

Finally, as shown in FIG. 4E, after the scarred and/or diseased portion 16a′″″ has been removed from the remainder of the cornea 16′″ of the eye 10′″″, the cross-linked donor corneal portion 20a′ is implanted into the eye 10′″ of the patient in the location previously occupied by the scarred and/or diseased corneal portion 16a″. Because the shape of the transplant corresponds to that of the removed portion 16a′″ of the cornea 16′″, the transplant sits comfortably in its position in the host cornea. As described above, after implantation of the cross-linked donor corneal portion 20a′ into the eye 10′″, sutures or a suitable adhesive (e.g., the biocompatible and biodegradable adhesive described above) may be utilized to secure the cross-linked donor corneal portion 20a′ in place on the host cornea 16″″ of the eye 10″. For example, if a biocompatible and biodegradable adhesive is used to secure the cross-linked donor corneal portion 20a′ in place in the cornea 16′″ of the eye 10′″, the edges of the donor corneal portion 20a′ are coated with the biocompatible and biodegradable adhesive so as to give the transplant a reliable stability. In this case, it is desirable to have the attachment of the transplant maintained by the biocompatible and biodegradable adhesive for a period of months (i.e., it is desirable for the transplant to be secured in place by the biocompatible and biodegradable adhesive for as long as possible).

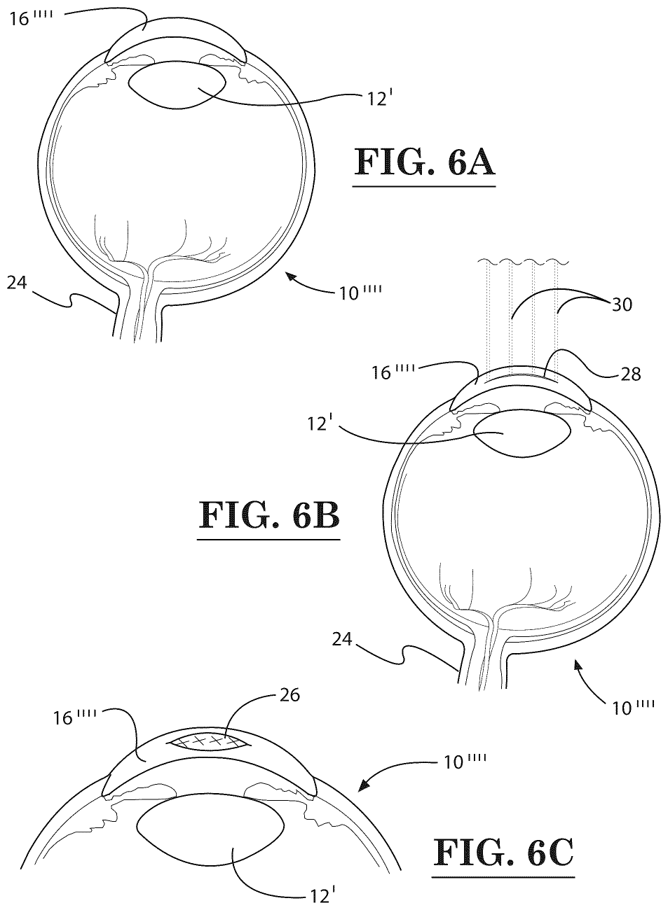

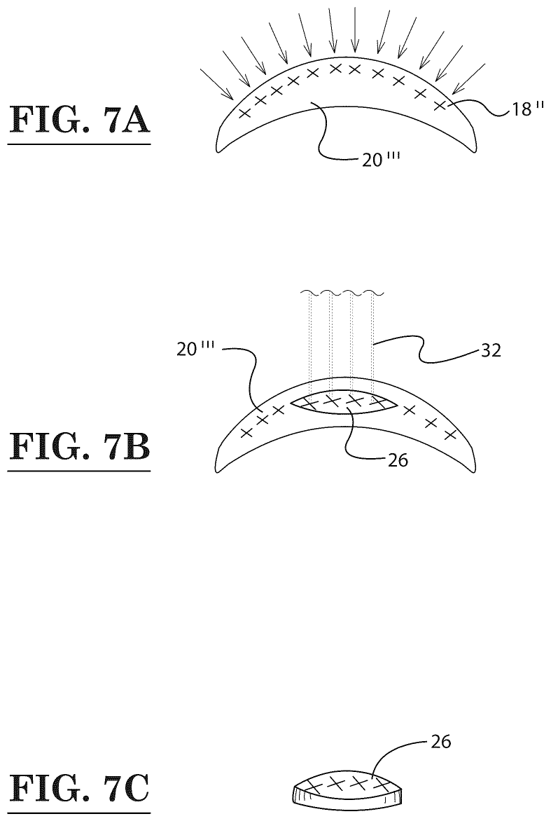

An illustrative embodiment of a corneal lenslet implantation procedure with a cross-linked cornea is shown in FIGS. 6A-6C and 7A-7C. Similar to the second, third, and fourth embodiments described above, FIGS. 6A-6C and 7A-7C illustrate a lamellar keratoplasty procedure wherein only a portion of the cornea 16″″ of the host eye 10″″ is removed during the procedure (i.e., a full-thickness corneal section is not removed). Although, the procedure of FIGS. 6A-6C and 7A-7C differs in several important respects from the above described procedures. In this embodiment, the corneal transplant is cross-linked in vitro. Then, using a femtosecond laser or an excimer laser, the surgeon carves out or ablates a three-dimensional (3D) corneal cross-linked augment from the donor cornea 20″″′ that exactly compensates for the refractive error of the recipient of the transplant. That is, the corneal cross-linked augment or inlay may be cut to the desired shape using a femtosecond laser, or the inlay may be shaped in vitro using an excimer laser prior to its implantation in the cornea 16″″ of the host eye 10″″. After making an internal pocket 28 in the recipient cornea 16′″″ of the host eye 10″″ with a femtosecond laser, the cross-linked transplant is folded and implanted in a predetermined fashion inside the host's corneal pocket 28 to provide stability to the eye 10″″ having keratoconus, keratoglobus, a thin cornea or abnormal corneal curvature, thereby preventing future corneal ectasia in this eye 10″″ and correcting its refractive errors. Advantageously, the procedure of this embodiment comprises a lamellar cross-linked corneal transplantation, which additionally results in simultaneous correction of the refractive error of the eye 10″″ of the patient. As used herein, the term “lenslet” refers to a lens implant configured to be implanted in a cornea of an eye. The lens implant may be formed from an organic material, a synthetic material, or a combination of organic and synthetic materials.

Now, with reference to FIGS. 6A-6C and 7A-7C, the illustrative embodiment will be described in further detail. The host eye 10″″ with lens 12′, cornea 16″″, and optic nerve 24 is shown in FIG. 6A, while the donor cornea 20′″″ is depicted in FIG. 7A. The donor cornea 20′″ of FIG. 7A may be a cross-linked cornea of a cadaver or a tissue culture-grown cornea that has been cross-linked. Turning to FIG. 6B, it can be seen that an internal corneal pocket 28 is created in the cornea 16″″ of the host eye 10″″ (e.g., by using a suitable laser, which is indicated diagrammatically in FIG. 6B by lines 30).

In FIG. 7A, the cross-linking 18″ of the donor cornea 20″″′ is diagrammatically illustrated. As mentioned in the preceding embodiments, it is to be understood that all or just a part of the donor cornea 20′″″ may be cross-linked. Then, after the donor cornea 20′″″ of FIG. 7A has been cross-linked (e.g., by using a photosensitizer in the form of riboflavin and UV radiation as described above), it can be seen that a cross-linked lamellar lenslet 26 is cut out from the remainder of the donor cornea 20′″ (e.g., by using a suitable laser, which is indicated diagrammatically in FIG. 7B by lines 32) such that it has the necessary shape for implantation into the host eye 10″″. As explained above, the cross-linked lamellar lenslet 26 may be cut from the donor cornea 20′″″ and appropriately shaped using a femtosecond laser or an excimer laser. The cross-linked lamellar lenslet 26 is capable of being prepared to any requisite shape using either the femtosecond laser or the excimer laser. FIG. 7C illustrates the shaped cross-linked lamellar lenslet 26 after it has been removed from the remainder of the donor cornea 20′″.

Finally, as shown in FIG. 6C, the cross-linked lamellar lenslet 26 is implanted into the cornea 16″″ of the host eye 10″″ of the patient in the location where the pocket 28 was previously formed. Because the shape of the transplant corresponds to that of the pocket 28 formed in the eye 10″″, the transplant sits comfortably in its position in the host cornea 16″″ As described above, after implantation of the cross-linked lamellar lenslet 26 into the eye 10″″ the refractive errors of the eye 10″″ have been corrected because the cross-linked lamellar lenslet 26 has been appropriately shaped to compensate for the specific refractive errors of the host eye 10″″ prior to its implantation into the eye 10″″. In addition, as explained above, the implantation of the cross-linked lamellar lenslet 26 provides additional stability to an eye having keratoconus, keratoglobus, a thin cornea, or abnormal corneal curvature.

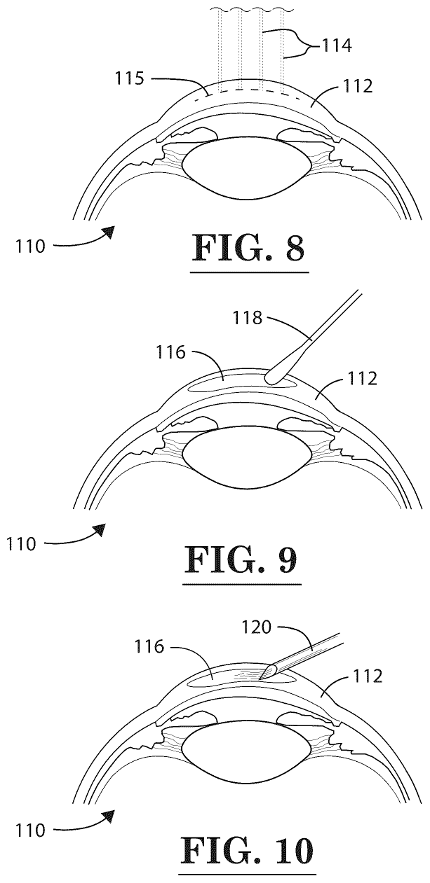

Another illustrative embodiment of a corneal lenslet implantation procedure with a cross-linked cornea is shown in FIGS. 8-14. In general, the procedure illustrated in these figures involves forming a two-dimensional cut into a cornea of an eye; creating a three-dimensional pocket in the cornea of the eye, cross-linking the interior stroma, and inserting a lenslet or lens implant into the three-dimensional pocket after the internal stromal tissue has been cross-linked.

Initially, in FIG. 8, the forming of a two-dimensional cut 115 into the cornea 112 of the eye 110 is diagrammatically illustrated. In particular, as shown in the illustrative embodiment of FIG. 8, the two-dimensional cut 115 is formed by making an intrastromal incision in the cornea 112 of the eye 110 using a femtosecond laser (i.e., the incision is cut in the cornea 112 using the laser beam(s) 114 emitted from the femtosecond laser). Alternatively, the two-dimensional cut 115 may be formed in the cornea 112 of the eye 110 using a knife.

Then, in FIG. 9, the forming of a three-dimensional corneal pocket 116 in the cornea 112 of the eye 110 is diagrammatically illustrated. In particular, as shown in the illustrative embodiment of FIG. 9, the three-dimensional corneal pocket 116 is formed by using a spatula 118. The formation of the intracorneal pocket 116 in the cornea 112 of the eye 110 allows one to gain access to the tissue surrounding the pocket 116 (i.e., the interior stromal tissue surrounding the pocket 116).

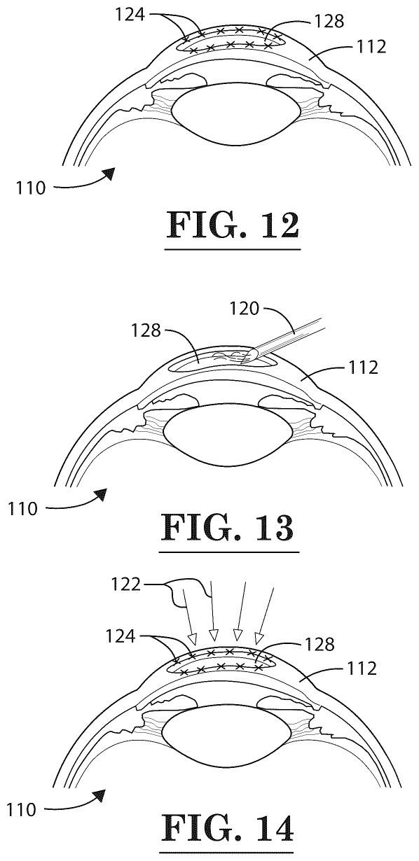

Turning again to FIGS. 8 and 9, in the illustrative embodiment, the corneal pocket 116 formed in the cornea 112 of the eye 110 may be in the form of an intrastromal corneal pocket cut into the corneal stroma. A femtosecond laser may be used to form a 2-dimensional cut into the cornea 112, which is then opened with a spatula 118 to create a 3-dimensional pocket 116. In one embodiment, a piece of the cornea 112 or a cornea which has a scar tissue is first cut with the femtosecond laser. Then, the cavity is cross-linked before filling it with an implant or inlay 128 to replace the lost tissue with a clear flexible inlay or implant 128 (see FIG. 12).

In one embodiment, a three-dimensional (3D) uniform circular, oval, or squared-shaped corneal pocket 116 is cut with a femtosecond laser and the tissue inside the pocket is removed to produce a three-dimensional (3D) pocket 116 to be cross-linked with riboflavin and implanted with a prepared implant.

After the pocket 116 is formed using the spatula 118, a photosensitizer is applied inside the three-dimensional pocket 116 so that the photosensitizer permeates the tissue surrounding the pocket 116 (see FIG. 10). The photosensitizer facilitates the cross-linking of the tissue surrounding the pocket 116. In the illustrative embodiment, the photosensitizer is injected with a needle 120 inside the stromal pocket 116 without lifting the anterior corneal stroma so as to cover the internal surface of the corneal pocket 116. In one or more embodiments, the photosensitizer or cross-linker that is injected through the needle 120 inside the stromal pocket comprises riboflavin, and/or a liquid suspension having nanoparticles of riboflavin disposed therein. Preferably, the cross-linker has between about 0.1% riboflavin to about 100% riboflavin therein (or between 0.1% and 100% riboflavin therein). Also, in one or more embodiments, an excess portion of the photosensitizer in the pocket 116 may be aspirated through the needle 120 until all, or substantially all, of the excess portion of the photosensitizer is removed from the pocket 116 (i.e., the excess cross-linker may be aspirated through the same needle so that the pocket 116 may be completely emptied or substantially emptied).

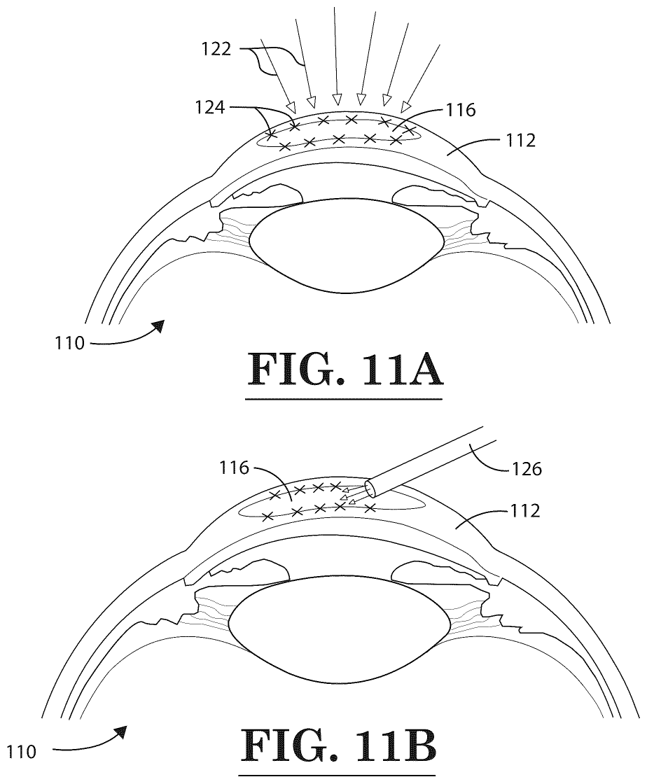

Next, turning to the illustrative embodiment of FIG. 11A, shortly after the photosensitizer is applied inside the pocket 116, the cornea 112 of the eye 110 is irradiated from the outside using ultraviolet (UV) radiation 122 so as to activate cross-linkers in the portion of the tissue surrounding the three-dimensional pocket 116, and thereby stiffen the cornea 112, prevent corneal ectasia of the cornea 112, and kill cells in the portion of the tissue surrounding the pocket 116. In the illustrative embodiment, the ultraviolet light used to irradiate the cornea 112 may have a wavelength between about 370 nanometers and about 380 nanometers (or between 370 nanometers and 380 nanometers). Also, in the illustrative embodiment, only a predetermined anterior stromal portion 124 of the cornea 112 to which the photosensitizer was applied is cross-linked (i.e., the surrounding wall of the corneal pocket 116), thereby leaving an anterior portion of the cornea 112 and a posterior stromal portion of the cornea 112 uncross-linked. That is, in the illustrative embodiment, the entire corneal area inside the cornea 112 exposed to the cross-linker is selectively cross-linked, thereby leaving the anterior part of the cornea 112 and the posterior part of the stroma uncross-linked. The portion of the cornea 112 without the cross-linker is not cross-linked when exposed to the UV radiation. In an alternative embodiment, the cornea 112 may be irradiated using wavelengths of light other than UV light as an alternative to, or in addition to being irradiated using the ultraviolet (UV) radiation 122 depicted in FIG. 11A. Also, microwave radiation may be used synergistically or additively to correct non-invasively the remaining refractive error(s) of the cornea.

Alternatively, as shown in FIG. 11B, a fiber optic 126 may be inserted into the corneal pocket 116 so as to apply the ultraviolet radiation and activate the photosensitizer in the wall of the corneal pocket 116. When the fiber optic 126 is used to irradiate the wall of the pocket 116, the ultraviolet radiation is applied internally, rather than externally as depicted in FIG. 11A.

Now, with reference to FIG. 12, it can be seen that, after the wall of the corneal pocket 116 has been stiffened and is devoid of cellular elements by the activation of the cross-linkers, a lens implant 128 is inserted into the corneal pocket 116 in order to change the refractive properties of the eye. In particular, in the illustrated embodiment, the lens implant 128 is inserted through a small incision, and into the corneal pocket 116, using forceps or microforceps. In one or more embodiments, the lens implant 128 that is inserted inside the pocket 116 in the cornea 112 is flexible and porous. Also, in one or more embodiments, the lens implant 128 may comprise a hybrid lens implant with an organic outer portion and a synthetic inner portion. The organic outer portion of the hybrid lens implant may be made from a transparent, hydrophilic organic polymer, while the synthetic inner portion of the hybrid lens implant may be made from a transparent, gas permeable, porous flexible polymer. For example, the transparent, hydrophilic polymer forming the organic outer portion may be formed from collagen, chitosan, poloxamer, polyethylene glycol, or a combination thereof (or any other transparent hydrophilic coating which can be deposited over the entire lens surface), while the flexible polymer forming the synthetic inner portion of the hybrid lens implant may be formed from silicone, acrylic, polymetacrylate, hydrogel, or a combination thereof. The surface of the lens implant 128 may have the appropriate shape to reshape the cornea 112 or the dioptric power to nullify the remaining spheric or astigmatic error of the eye. More particularly, in one or more embodiments, the lens implant 128 may have one of: (i) a concave surface to correct myopic refractive errors (i.e., a minus lens for correcting nearsightedness), (ii) a convex surface to correct hyperopic refractive errors (i.e., a plus lens for correcting farsightedness), or (iii) a toric shape to correct astigmatic refractive errors.

In the illustrative embodiment, the irradiation of the cornea 112 using the ultraviolet (UV) radiation 122 only activates cross-linkers in the portion of the stromal tissue surrounding the three-dimensional pocket 116, and only kills the cells in the portion of the tissue surrounding the pocket 116, so as to leave only a thin layer of cross-linked collagen to prevent an immune response and rejection of the lens implant 128 and/or encapsulation by fibrocytes, while preventing post-operative dry eye formation. In addition to preventing encapsulation of the lens implant 128 by fibrocytes, the cross-linking of the stromal tissue surrounding the pocket 116 also advantageously prevents corneal haze formation around the lens implant 128. That is, the cross-linking of the stromal tissue surrounding the lens implant 128 prevents formation of myofibroblast from surrounding keratocytes, which then convert gradually to fibrocytes that appear as a haze, and then white encapsulation inside the cornea, thereby causing light scattering in front of the patient's eye.

As shown in FIGS. 13 and 14, the crosslinking procedure described above may be repeated after the lens implant 128 is implanted so as to prevent any cellular invasion in the area surrounding the implant 128. Initially, with reference to FIG. 13, the photosensitizer is reinjected inside the space between the lens implant 128 and the surrounding corneal tissue using a needle 120. In one or more embodiments, the needle 120 for injecting the photosensitizer may comprise a 30-32 gauge needle. Then, after the reinjection of the cross-linker, the cornea 112 is re-irradiated with ultraviolet radiation 122 to cross-link the tissue surrounding the lens implant 128 so as to prevent cellular migration towards the lens implant 128 (see FIG. 14).

In one or more embodiments, the lens implant or inlay 128 may be prepared ahead of time with known techniques, wherein the inlay 128 may be coated with a biocompatible material, such as collagen, elastin, polyethylene glycol, biotin, streptavidin, etc., or a combination thereof. The inlay 128 and the coating may be cross-linked with a photosensitizer or cross-linker, such as riboflavin, prior to being implanted into the pocket 116 in the cornea 112 of the eye.

In another embodiment, the lens implant or inlay 128 may be silicone, methacrylate, hydroxyethylmethacrylate (HEMA), or any other biocompatible transparent material, or a mixture thereof. The lens implant or inlay 128 also may be coated with materials, such as collagen or elastin, and may have a desired thickness of from 2 microns to 70 microns or more.

In yet another embodiment, the lens implant or inlay 128 is formed from an eye bank cornea, or a cross-linked eye bank cornea, etc. In general, there is a tremendous paucity of normal cadaver corneas for total or partial implants, such as for a corneal transplant of a corneal inlay. Because all the cellular elements are killed during the crosslinking of the corneal inlay, and because the corneal collagen is cross-linked and denatured, the remaining collagenous elements are not immunogenic when implanted inside the body or in the cornea of a patient. Advantageously, the prior cross-linking of the organic material, such as in the cadaver cornea, permits transplantation of the corneal inlay from an animal or human cornea or any species of animal to another animal or human for the first time without inciting a cellular or humoral response by the body, which rejects the inlay. Thus, cross-linking transparent cadaveric tissue for corneal transplantation, or as an inlay to modify of the refractive power of the eye, is highly beneficial to many patients who are on the waiting list for a corneal surgery. In addition, the surgery may be planned ahead of time without necessitating the urgency of the surgery when a fresh cadaver eye becomes available. In one or more embodiments, the collagens may be driven from the animal cornea, and cross-linked. Also, in one or more embodiments, the implant or inlay 128 may be made of cross-linked animal cornea or human cornea that is cut using a femtosecond laser to any desired shape and size, and then ablated with an excimer laser or cut with a femtosecond laser to a have a desired refractive power.

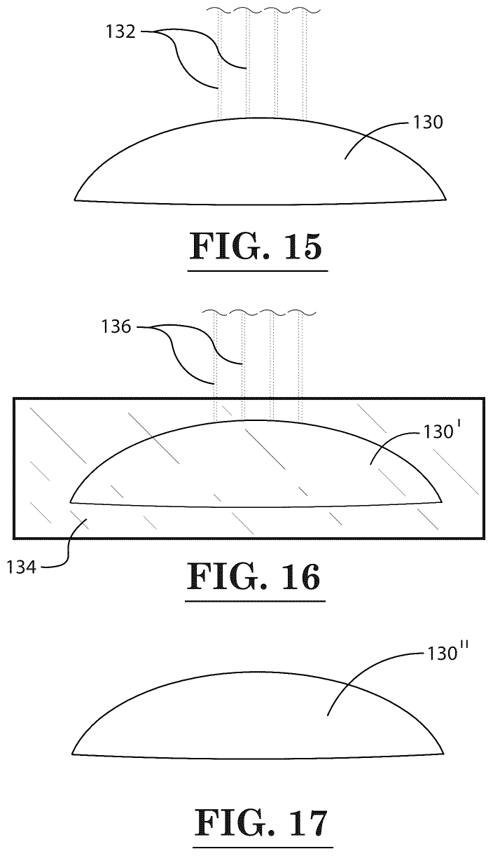

For example, as shown in FIG. 15, the lens implant or inlay 130 may be formed from an organic block of a polymer (e.g., donor cornea) by cutting the lens implant 130 using an excimer laser (e.g., by using the laser beam(s) 132 emitted from the excimer laser). Alternatively, referring to FIG. 16, the lens implant or inlay 130′ may be formed from an organic block 134 of a polymer (e.g., donor cornea) by cutting the lens implant 130′ from the block 134 using a femtosecond laser or a computerized femto-system (e.g., by using the laser beam(s) 136 emitted from the femtosecond laser).

In still another embodiment, as depicted in FIG. 17, the lens implant or inlay 130″ is made using three-dimensional (3D) printing technology or a molding technique in order to form the lens implant or inlay 130″ into the desired shape, size or thickness. The transparent material of the 3D-printed implant or inlay 130″ may be coated with one or more biocompatible polymers and cross-linked prior to the implantation.

In yet another embodiment, after the implantation of an intraocular lens, the remaining refractive error of the eye may be corrected by the implantation of a lens implant or inlay 128 in the cross-linked pocket 116 of the cornea 112, thereby eliminating the need for entering the eye cavity to replace the original intraocular lens.

In still another embodiment, the remaining refractive error of the eye is corrected after an intraocular lens implantation by placing an inlay 128 on the surface of the cornea 112 of the patient while the shape of the cornea 112 is corrected with an excimer laser and wavefront optimized technology so that the patient is provided instant input on its effect on his or her vision. In this embodiment, an inlay similar to a contact lens is placed on the cornea 112 that, after correction, matches the desired refractive correction of the eye, and then, subsequently, the inlay 128 is implanted inside the cross-linked corneal pocket 116.

In yet another embodiment, the implant or inlay 128 may be ablated with an excimer laser for implantation in the cross-linked pocket 116, or after cross-linking the exposed corneal stroma in LASIK surgery.

In still another embodiment, a small amount of hyaluronic acid or a viscous fluid is injected into the pocket 116 prior to the implantation of the implant or inlay 128 so as to simplify the insertion of the implant or inlay 128 in the corneal pocket 116.

In yet another embodiment, the implant or inlay 128 is prepared having four marking holes of 0.1-2 millimeter (mm) in diameter in the inlay periphery at an equally sized distances so that the implant 128 may be rotated with a hook, if desired, after the implantation as needed to match the axis of an astigmatic error of the eye during the surgery as measured simultaneously with a wavefront technology system, such as an Optiwave Refractive Analysis (ORA) system or Holos® system, which are commercially available for measurement of astigmatism or its axis.

In still another embodiment, the implant or inlay 128 is located on the visual axis and may provide 1 to 3 times magnification for patients whose macula is affected by a disease process needing magnifying glasses for reading, such as in age-related macular degeneration, macular edema, degenerative diseases of the retina, etc. Because these eyes cannot be used normally for reading without external magnifier glasses, providing magnification by a corneal implant to one eye assists the patients in being able to read with one eye and navigate the familiar environment with their other eye.

In yet another embodiment, the surface of the cornea 112 is treated after surgery in all cases daily with an anti-inflammatory agent, such as steroids, nonsteriodal anti-inflammatory drugs (NSAIDs), immune-suppressants, such as cyclosporine A or mycophenolic acid, anti-proliferative agents, antimetabolite agents, or anti-inflammatory agents (e.g., steroids, NSAIDS, or antibiotics etc.) to prevent inflammatory processes after the corneal surgery, inlay implantation or crosslinking, while stabilizing the integrity of the implant 128 and preventing future cell growth in the organic implant or the adjacent acellular corneal tissue. In this embodiment, the medication is injected in the corneal pocket 116 along with the implantation or the implant 128 is dipped in the medication first, and then implanted in the cross-linked corneal pocket 116.

In still another embodiment, a cross-linked corneal inlay is placed over the cross-linked corneal stroma after a LASIK incision, and is abated to the desired size with an excimer laser using a topography guided ablation. By means of this procedure, the refractive power of the eye is corrected, while simultaneously providing stability to an eye prone to conceal ectasia postoperatively after a LASIK surgery. Then, the LASIK flap is placed back over the implant.

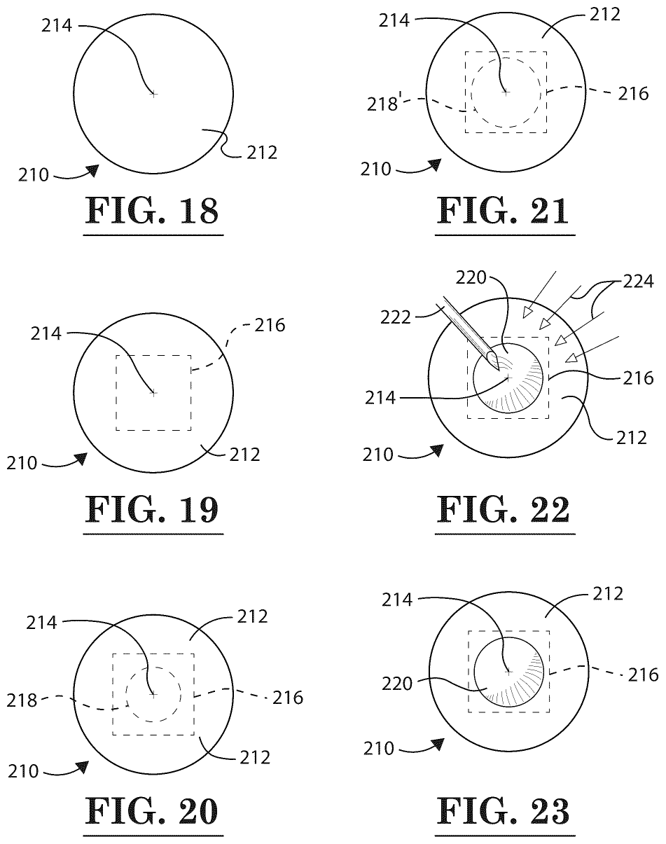

Yet another illustrative embodiment of a corneal lenslet implantation procedure with a cross-linked cornea is shown in FIGS. 18-23. In general, the procedure illustrated in these figures involves initially making an intrastromal square pocket surrounding the visual axis of the eye, and then, after forming the initial square pocket, a three-dimensional circular portion of diseased or weak stromal tissue is cut, removed, and replaced with a circular implant which fits into the circle that borders the four sides of the square. A front view of the cornea 212 of the eye 210 with the centrally-located visual axis 214 is illustrated in FIG. 18. Advantageously, in the illustrative embodiment of FIGS. 18-23, corneal tissue removal around the visual axis is greatly facilitated, and nearly perfect centration of the lens implant or inlay 220 about the visual axis is possible because the lens implant 220 fits within a depressed circular recess at the bottom of the pocket 216. As such, the undesirable decentering of the lens implant is prevented.

Initially, in FIG. 19, the forming of an intrastromal square-shaped pocket 216 surrounding the visual axis 214 (represented by a plus sign) in the cornea 212 of the eye 210 is diagrammatically illustrated. In particular, as shown in the illustrative embodiment of FIG. 19, the square-shaped pocket 216 is formed by making a two-dimensional intrastromal incision in the cornea 212 of the eye 210 using a femtosecond laser (i.e., the incision is cut in the cornea 212 using the laser beam(s) emitted from the femtosecond laser).

Then, in FIG. 20, the removal of a three-dimensional circular portion 218 of diseased or weak stromal tissue in the cornea 212 of the eye 210 is diagrammatically illustrated. In particular, as shown in the illustrative embodiment of FIG. 20, the three-dimensional circular stromal tissue portion 218 has a first diameter, which is less than a width of the square-shaped pocket 216 so that the three-dimensional circular stromal tissue portion 218 is disposed within the boundaries of the square-shaped pocket 216. The three-dimensional circular stromal tissue portion 218′ depicted in FIG. 21 is generally similar to that illustrated in FIG. 20, except that the three-dimensional circular stromal tissue portion 218′ depicted in FIG. 21 has a second diameter that is slightly larger than the first diameter of the three-dimensional circular stromal tissue portion 218 in FIG. 20. As such, the periphery of the three-dimensional circular stromal tissue portion 218′ depicted in FIG. 21 is disposed closer to the square-shaped pocket 216, but still within the confines of the square-shaped pocket 216. In the illustrative embodiment, the three-dimensional circular stromal tissue portion 218, 218′ may be removed using forceps or micro-forceps. In an exemplary embodiment, the diameter of the circular stromal tissue portion 218, 218′ that is removed from the cornea 212 is between approximately 5 millimeters and approximately 8 millimeters, inclusive (or between 5 millimeters and 8 millimeters, inclusive).

In an alternative embodiment of the corneal lenslet implantation procedure, three (3) sequential cuts may be made in the stromal portion of the cornea 212 of the eye 210 using a femtosecond laser in order to form the pocket. First, a lower circular cut or incision centered about the visual axis (i.e., a lower incision with the patient in a supine position) is made using the femtosecond laser. Then, a second vertical cut is made above the lower incision using the femtosecond laser to form the side(s) of a circular cutout portion. Finally, a third square or circular cut (i.e., an upper incision) is made above the vertical cut using the femtosecond laser. In the illustrative embodiment, the lower incision is parallel to the upper incision, and the vertical cut extends between lower incision and the upper incision. In this alternative embodiment, the three-dimensional circular stromal tissue cutout portion bounded by the lower incision on the bottom thereof, the vertical cut on the side(s) thereof, and the upper incision on the top thereof is removed from the cornea 212 of the eye 210 using a pair of forceps. A cavity formed by the upper incision facilitates the removal of the three-dimensional circular stromal tissue cutout portion. As described above, the third cut or incision formed using the femtosecond laser may be an upper circular cut that is larger than the lower circular cut, rather than an upper square cut that is larger than the lower circular cut.

Turning to FIG. 22, after the three-dimensional circular stromal tissue portion 218, 218′ is removed, a photosensitizer is applied inside the pocket 216 so that the photosensitizer permeates the tissue surrounding the pocket 216. The photosensitizer facilitates the cross-linking of the tissue surrounding the pocket 216. In the illustrative embodiment, the photosensitizer is injected with a needle 222 inside the stromal pocket 216. In one or more embodiments, the photosensitizer or cross-linker that is injected through the needle 222 inside the stromal pocket 216 comprises riboflavin, and/or a liquid suspension having nanoparticles of riboflavin disposed therein. Preferably, the cross-linker has between about 0.1% riboflavin to about 100% riboflavin therein (or between 0.1% and 100% riboflavin therein). Also, in one or more embodiments, an excess portion of the photosensitizer in the pocket 216 may be aspirated through the needle 222 until all, or substantially all, of the excess portion of the photosensitizer is removed from the pocket 216 (i.e., the excess cross-linker may be aspirated through the same needle 222 so that the pocket 216 may be completely emptied or substantially emptied).

Next, turning again to the illustrative embodiment of FIG. 22, shortly after the photosensitizer is applied inside the pocket 216, the cornea 212 of the eye 210 is irradiated from the outside using ultraviolet (UV) radiation 224 so as to activate cross-linkers in the portion of the tissue surrounding the three-dimensional pocket 216, and thereby stiffen the cornea 212, prevent corneal ectasia of the cornea 212, and kill cells in the portion of the tissue surrounding the pocket 216. In the illustrative embodiment, the ultraviolet light used to irradiate the cornea 212 may have a wavelength between about 370 nanometers and about 380 nanometers (or between 370 nanometers and 380 nanometers). Also, in the illustrative embodiment, only a predetermined anterior stromal portion of the cornea 212 to which the photosensitizer was applied is cross-linked (i.e., the surrounding wall of the corneal pocket 216), thereby leaving an anterior portion of the cornea 212 and a posterior stromal portion of the cornea 212 uncross-linked. That is, in the illustrative embodiment, the entire corneal area inside the cornea 212 exposed to the cross-linker is selectively cross-linked, thereby leaving the anterior part of the cornea 212 and the posterior part of the stroma uncross-linked. The portion of the cornea 212 without the cross-linker is not cross-linked when exposed to the UV radiation. In an alternative embodiment, the cornea 212 may be irradiated using wavelengths of light other than UV light as an alternative to, or in addition to being irradiated using the ultraviolet (UV) radiation 224 depicted in FIG. 22. Also, microwave radiation may be used synergistically or additively to correct non-invasively the remaining refractive error(s) of the cornea. In addition, in an alternative embodiment, the ultraviolet (UV) radiation may be applied after the implantation of the lens implant 220 to perform the crosslinking, rather than before the implantation of the lens implant 220 as described above. Further, rather than applying the ultraviolet (UV) radiation from outside the cornea 212, the stromal tissue of the pocket 216 may be irradiated from inside by means of a fiber optic, before or after the implantation of the lens implant 220.

Now, with combined reference to FIGS. 22 and 23, it can be seen that, before or after the wall of the corneal pocket 216 has been stiffened and is devoid of cellular elements by the activation of the cross-linkers, a circular lens implant 220 is inserted into the circular recess at the bottom of the pocket 216 formed by the three-dimensional circular stromal tissue cutout portion 218, 218′ that was removed. That is, the circular lens implant 220 fits within the periphery of the circular recess that borders the four sides of the squared-shaped pocket 216. In particular, in the illustrated embodiment, the circular lens implant 220 is inserted through a small incision, and into the circular recess at the bottom of the pocket 216 using forceps or microforceps. In the illustrative embodiment, the flexible lens implant 220 may be folded, inserted through the small incision, placed inside the circular recess at the bottom of the pocket 216, and finally unfolded through then small incision. In one or more embodiments, the lens implant 220 that is inserted inside the pocket 216 in the cornea 212 is flexible and porous. Also, in one or more embodiments, the lens implant 220 may comprise a hybrid lens implant with an organic outer portion and a synthetic inner portion. The organic outer portion of the hybrid lens implant may be made from a transparent, hydrophilic organic polymer, while the synthetic inner portion of the hybrid lens implant may be made from a transparent, gas permeable, porous flexible polymer. For example, the transparent, hydrophilic polymer forming the organic outer portion may be formed from collagen, chitosan, poloxamer, polyethylene glycol, or a combination thereof (or any other transparent hydrophilic coating which can be deposited over the entire lens surface), while the flexible polymer forming the synthetic inner portion of the hybrid lens implant may be formed from silicone, acrylic, polymetacrylate, hydrogel, or a combination thereof.

Advantageously, the lens implant 220 of the aforedescribed illustrative embodiment always remains perfectly centered around the visual axis 214 of the eye 210, and will not move because it is disposed within the circular recess at the bottom of the pocket 216. As explained above, the lens implant 220 may be formed from an organic material, synthetic material, polymeric material, and combinations thereof. The lens implant 220 may replace either a diseased tissue or create a new refractive power for the eye 210, as explained hereinafter.

In the illustrative embodiment, the lens implant 220 may correct the refractive errors of the eye 210. The refractive error correction may be done by the lens implant 220 having a curvature that changes the corneal surface of the cornea 212. Alternatively, the lens implant 220 may have a different index of refraction that corrects the refractive power of the cornea 212. In the illustrative embodiment, the lens implant 220 may have the appropriate shape to reshape the cornea 212 or the dioptric power to nullify the remaining spheric or astigmatic error of the eye. More particularly, in one or more embodiments, the lens implant 220 may have one of: (i) a concave anterior surface to correct myopic refractive errors (i.e., a minus lens for correcting nearsightedness), (ii) a convex anterior surface to correct hyperopic refractive errors (i.e., a plus lens for correcting farsightedness), or (iii) a toric shape to correct astigmatic refractive errors.

In the illustrative embodiment, the irradiation of the cornea 212 using the ultraviolet (UV) radiation 224 only activates cross-linkers in the portion of the stromal tissue surrounding the three-dimensional pocket 216, and only kills the cells in the portion of the tissue surrounding the pocket 216, so as to leave only a thin layer of cross-linked collagen to prevent an immune response and rejection of the lens implant 220 and/or encapsulation by fibrocytes, while preventing post-operative dry eye formation. In addition to preventing encapsulation of the lens implant 220 by fibrocytes, the cross-linking of the stromal tissue surrounding the pocket 216 also advantageously prevents corneal haze formation around the lens implant 220. That is, the cross-linking of the stromal tissue surrounding the lens implant 220 prevents formation of myofibroblast from surrounding keratocytes, which then convert gradually to fibrocytes that appear as a haze, and then white encapsulation inside the cornea, thereby causing light scattering in front of the patient's eye.

It is readily apparent that the aforedescribed corneal transplant procedures offer numerous advantages. First, the implementation of the aforedescribed corneal transplant procedures reduces the likelihood that the implanted cornea will be rejected by the patient. Secondly, the aforedescribed corneal transplant procedures enable the clarity of the transplanted cornea to be preserved. Finally, the aforedescribed corneal transplant procedures reduce the likelihood that the transplanted cornea will be invaded by migrating cells, such as migrating cells that might initiate an immune response such as macrophage, lymphocytes or leucocytes or vascular endothelial cells. These types of migrating cells are discouraged by the cross-linked corneal collagen which does not provide an easily accessible tissue to invade. In addition, the use of above described tissue adhesives reduces the surgical procedure significantly. Moreover, the aforedescribed corneal lenslet implantation procedures modify the cornea so as to better correct ametropic conditions. Furthermore, the corneal lenslet implantation procedures described above prevent the lens implant from moving around inside the cornea once implanted, thereby ensuring that the lens implant remains centered about the visual axis of the eye.

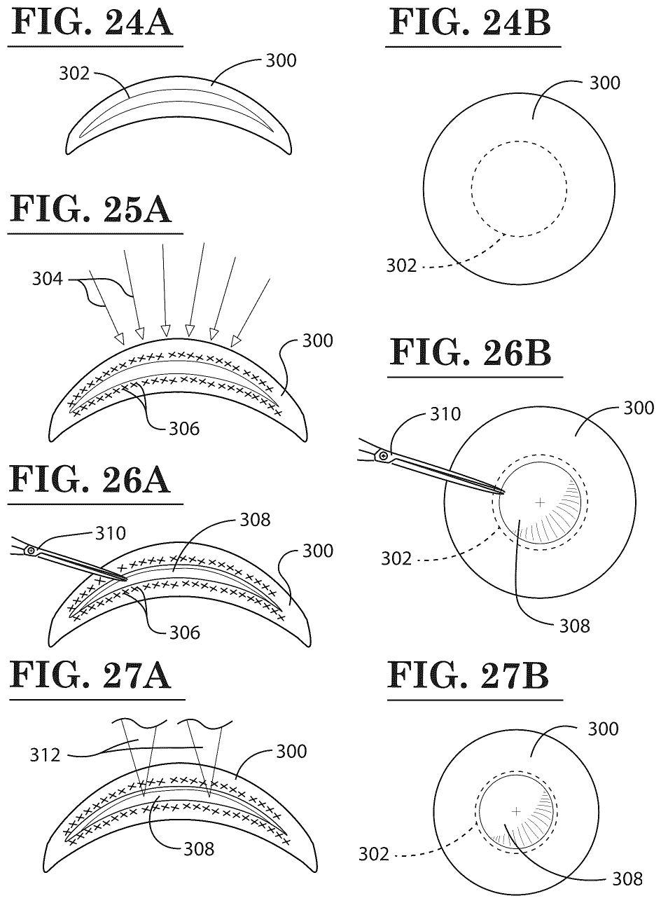

With reference to the embodiment of FIGS. 24A-27B, a first illustrative intracorneal lens implantation procedure with a cross-linked cornea will be explained. In general, the procedure illustrated in these figures involves forming a pocket in the cornea of an eye, cross-linking the interior stroma, inserting a lens implant into the pocket, and then applying laser energy to the lens implant in the pocket using a laser to correct refractive errors of the lens implant and/or the eye in a non-invasive manner. In this embodiment, no flap is formed in the cornea 300 of the eye.

In FIGS. 24A and 24B, the forming of a corneal pocket 302 in the cornea 300 of the eye is diagrammatically illustrated. FIG. 24A illustrates a cross-sectional view of the eye, whereas FIG. 24B illustrates a front view of the eye. The formation of the intracorneal pocket 302 in the cornea 300 of the eye allows one to gain access to the tissue bounding the pocket 302 (i.e., the interior stromal tissue bounding the pocket 302). In particular, as shown in the illustrative embodiment of FIG. 24A, the pocket 302 is formed by making an intrastromal incision in the cornea 300 of the eye using either a femtosecond laser (i.e., the incision is cut in the cornea 300 using the laser beam(s) emitted from the femtosecond laser) or a mechanical keratome (e.g., a mechanical microkeratome).

After the pocket 302 is cut using the femtosecond laser or mechanical keratome, a photosensitizer is applied inside the pocket so that the photosensitizer permeates the tissue bounding the pocket 302. The photosensitizer facilitates the cross-linking of the tissue bounding the pocket 302. In the illustrative embodiment, the photosensitizer is injected with a needle inside the stromal pocket without lifting the anterior corneal stroma so as to cover the internal surface of the corneal pocket 302 (e.g., as shown in FIG. 10). In one or more embodiments, the photosensitizer or cross-linker that is injected through the needle inside the stromal pocket comprises riboflavin, and/or a liquid suspension having nanoparticles of riboflavin disposed therein. Preferably, the cross-linker has between about 0.1% riboflavin to about 100% riboflavin therein (or between 0.1% and 100% riboflavin therein). Also, in one or more embodiments, an excess portion of the photosensitizer in the pocket 302 may be aspirated through the needle until all, or substantially all, of the excess portion of the photosensitizer is removed from the pocket 302 (i.e., the excess cross-linker may be aspirated through the same needle so that the pocket 302 may be completely emptied or substantially emptied).

Next, turning to the illustrative embodiment of FIG. 25A, shortly after the photosensitizer is applied inside the pocket, the cornea 300 of the eye is irradiated from the outside using ultraviolet (UV) radiation 304 so as to activate cross-linkers in the portion of the tissue bounding the pocket 302, and thereby stiffen the cornea 300, prevent corneal ectasia of the cornea 300, and kill cells in the portion of the tissue bounding the pocket 302. In the illustrative embodiment, the ultraviolet light used to irradiate the cornea 300 may have a wavelength between about 370 nanometers and about 380 nanometers (or between 370 nanometers and 380 nanometers). Also, in the illustrative embodiment, only a predetermined anterior stromal portion 306 of the cornea 300 to which the photosensitizer was applied is cross-linked (i.e., the bounding wall of the corneal pocket 302), thereby leaving an anterior portion of the cornea 300 and a posterior stromal portion of the cornea 300 uncross-linked. That is, in the illustrative embodiment, the entire corneal area inside the cornea 300 exposed to the cross-linker is selectively cross-linked, thereby leaving the anterior part of the cornea 300 and the posterior part of the stroma uncross-linked. The portion of the cornea 300 without the cross-linker is not cross-linked when exposed to the UV radiation. In an alternative embodiment, the cornea 300 may be irradiated using microwaves as an alternative to, or in addition to being irradiated using the ultraviolet (UV) radiation 304 depicted in FIG. 25A.

Now, with reference to FIGS. 26A and 26B, it can be seen that, after the cornea 300 has been stiffened and is devoid of cellular elements by the activation of the cross-linkers, a lens implant 308 is inserted into the corneal pocket 302 in order to change the refractive properties of the eye. FIG. 26A illustrates a cross-sectional view of the eye depicting the implantation of the intracorneal lens implant 308, whereas FIG. 26B illustrates a front view of the eye depicting the implantation of the intracorneal lens implant 308. In particular, in the illustrated embodiment, the lens implant 308 is inserted through a small incision, and into the corneal pocket 302, using forceps or microforceps 310. In one or more embodiments, the lens implant 308 that is inserted inside the pocket 302 in the cornea 300 is flexible and porous. Also, in one or more embodiments, the lens implant 308 may comprise a hybrid lens implant with an organic outer portion and a synthetic inner portion. The organic outer portion of the hybrid lens implant may be made from a transparent, hydrophilic organic polymer, while the synthetic inner portion of the hybrid lens implant may be made from a transparent, gas permeable, porous flexible polymer. For example, the transparent, hydrophilic polymer forming the organic outer portion may be formed from collagen, chitosan, poloxamer, polyethylene glycol, or a combination thereof (or any other transparent hydrophilic coating which can be deposited over the entire lens surface), while the flexible polymer forming the synthetic inner portion of the hybrid lens implant may be formed from silicone, acrylic, polymetacrylate, hydrogel, or a combination thereof. The surface of the lens implant 308 may have the appropriate shape to reshape the cornea 300 or the dioptric power to nullify the remaining spheric or astigmatic error of the eye. More particularly, in one or more embodiments, the lens implant 308 may have one of: (i) a concave surface to correct myopic refractive errors (i.e., a minus lens for correcting nearsightedness), (ii) a convex surface to correct hyperopic refractive errors (i.e., a plus lens for correcting farsightedness), or (iii) a toric shape to correct astigmatic refractive errors. In one or more embodiment, the lens implant 308 may have any suitable shape (e.g., circular, annular, etc.) for correcting a particular error of the eye, and may be implanted in any suitable location within the cornea 300 for correcting the particular error of the eye.

In the illustrative embodiment, the irradiation of the cornea 300 using the ultraviolet (UV) radiation 304 only activates cross-linkers in the portion of the stromal tissue bounding the pocket 302, and only kills the cells in the portion of the tissue bounding the pocket 302, so as to leave only a thin layer (e.g., between 20 and 30 microns) of cross-linked collagen to prevent rejection of the lens implant 308 and/or encapsulation by fibrocytes, while preventing post-operative dry eye formation. In addition to preventing encapsulation of the lens implant 308 by fibrocytes, the cross-linking of the stromal tissue bounding the pocket 302 also advantageously prevents corneal haze formation around the lens implant 308. That is, the cross-linking of the stromal tissue surrounding the lens implant 308 prevents formation of myofibroblast from surrounding keratocytes, which then convert gradually to fibrocytes that appear as a haze, and then white encapsulation inside the cornea, thereby causing light scattering in front of the patient's eye.

In one or more further embodiments, after the lens implant 308 has been inserted into the pocket 302, an additional amount of photosensitizer (e.g., an additional amount of riboflavin) is injected into the pocket 302, and the cornea 300 is irradiated an additional time so as to further stiffen stromal tissue of the cornea and expand the area of acellular collagenous stromal tissue surrounding the lens implant 308 to prevent rejection of the lens implant 308 and/or encapsulation of the lens implant 308 by fibrocytes, while preventing post-operative dry eye formation. That is, the area of acellular collagenous stromal tissue surrounding the lens implant 308 is able to be cross-linked repeatedly through the use of additional riboflavin injections so that the area of intrastromal crosslinking may be extended, and to prevent implant rejection and cellular fibrosis formation at any time after the initial procedure. This additional cross-linking still leaves the anterior stromal nerves intact and uncross-linked so as to not produce dry eye formation.

Referring again to the illustrative embodiment of FIGS. 24A-27B, after the lens implant 308 has been inserted into the pocket 302 in the cornea 300 of the eye, laser energy is applied to the lens implant 308 in the pocket 302 using a laser 312 so as to correct refractive errors of the lens implant 308 and/or the eye in a non-invasive manner (refer to FIG. 27A). In the illustrative embodiment, a two-photon or multi-photon laser 312 is used to apply the laser energy to the lens implant 308 in the pocket 302 so as to modify the index of refraction of a discrete internal part of the lens implant 308 in a non-invasive manner, while preventing post-operative dry eye formation. In the illustrative embodiment, the laser energy applied by the two-photon or multi-photon laser has a predetermined energy level below an optical breakdown power level of the two-photon or multi-photon laser. The fast-acting, short laser pulse of the two-photon or multi-photon laser 312 is used to modify the refractive power of the lens implant 308. In the illustrative embodiment, the two-photon or multi-photon laser 312 is not used to modify the shape of the lens implant. In the illustrative embodiment, the multi-photon laser may comprise a three-photon laser, etc. Also, in the illustrative embodiment, the two-photon or multi-photon laser is a specific type of femtosecond laser.

In the illustrative embodiment, the laser beam(s) emitted by the two-photon or multi-photon laser 312 heats up the lens implant 308, and thereby modifies the index of refraction of the lens implant 308 (i.e., it creates a more positive or negative lens). Because a two-photon or multi-photon laser 312 comprises two or more laser beams that come together at the focal point of the laser, less energy is passing through the anterior corneal tissue disposed in front of the lens implant 308. Thus, advantageously, in the illustrative embodiment, the two-photon or multi-photon laser 312 does not damage the surface of the cornea or the corneal tissue anteriorly disposed relative to the lens implant 308. In the illustrative embodiment, the two-photon or multi-photon laser 312 modifies the interior of the lens implant 308 (i.e., by modifying its refractive index), but it does not modify the surface of the lens implant 308 or the corneal tissue disposed anteriorly disposed relative to the lens implant 308. In the illustrative embodiment, the laser beam(s) of the two-photon or multi-photon laser 312 may have a wavelength between about 700 nanometers and about 1100 nanometers (or between 700 nanometers and 1100 nanometers). In the illustrative embodiment, the two-photon or multi-photon laser 312 does not require a photosensitizer, and the laser beams emitted thereby may penetrate between 100 and 400 microns into the interior of the cornea.

In one or more embodiments, prior to the application of the laser energy to the lens implant 308 in the pocket 302 by the two-photon or multi-photon laser 312, a virtual model of the lens implant 308 is generated, and the two-photon or multi-photon laser 312 is focused in accordance with the virtual model. In particular, a specially programmed data processing device (i.e., a specially programmed computing device or computer) is used to generate a virtual model of the lens implant 308 so that a new index of refraction of the lens implant 308 at the focal point of the two-photon or multi-photon laser 312 is capable of being determined prior to the application of the two-photon or multi-photon laser 312. Then, the specially programmed data processing device (i.e., a specially programmed computing device or computer) is used to focus the two-photon or multi-photon laser 312 non-invasively outside the eye in accordance with the virtual model generated for the lens implant 308.

In one or more further embodiments, a femtosecond laser, a two-photon laser, or a multi-photon laser may be used to apply laser energy to the lens implant 308 in the pocket 302 in order to increase the index of refraction of a particular area of the lens implant (e.g., by creating a prismatic line on the surface of the lens or inside of the lens), and thereby convert the lens implant from a monofocal lens to a bifocal lens or trifocal lens. In these further embodiments, the particular area of the lens implant 308 that the index of refraction is increased may comprise one of: (i) an area slightly below the cornea or the central visual axis of the eye, (ii) a central area centrally located on the central visual axis of the eye, and (iii) a peripheral area circumscribing the central visual axis of the eye. For example, in one embodiment, the particular area of the lens implant 308 that is modified may be 2-3 mm in diameter to correct presbyopia in an older person. The index of refraction of the particular area of the lens implant 308 may be modified to correct myopic refractive errors (i.e., nearsightedness), hyperopic refractive errors (i.e., farsightedness), or astigmatic refractive errors. Because the lens implant 308 can be removed from the eye (e.g., using a spatula), and replaced, the entire refractive error correction process described above can be reversible, and is capable of being repeated.

Also, in one or more further embodiments, a femtosecond laser, a two-photon laser, or a multi-photon laser may be used to apply laser energy to the lens implant 308 in the pocket 302 in order to create diffractive portions within the lens implant 308, thereby resulting in a bifocal lens comprising both refractive and diffractive lens portions.

In the method described above, as illustrated in FIGS. 24A-27B, a photorefractive keratectomy (PRK) procedure is not performed on the front surface of the cornea 300 so that the front surface of the cornea 300 is not required to be ablated by an excimer laser. Also, a laser-assisted in situ keratomileusis (LASIK) procedure is not performed on the cornea 300 of FIGS. 24A-27B so that a flap is not required to be formed in the cornea 300, thereby preventing a formation of dry eye in a patient resulting from the severing of the corneal nerves supplying the front surface of the cornea 300. That is, with the method described above, it is not necessary to form a LASIK flap, which requires severing the corneal nerves about a 300 degree area of the cornea. In some patients, it can take over a year to recover from the dry eye that results from the formation of the flap during the LASIK procedure.

In a second illustrative embodiment of the intracorneal lens implantation procedure with the cross-linking of the cornea, a lens implant is soaked in a crosslinking solution prior to be inserted into the eye of the patient. As will be described in further detail hereinafter, this method generally includes soaking a lens implant in a crosslinking solution, forming a pocket in the cornea of an eye, inserting the lens implant in the pocket, cross-linking the interior stroma of the cornea, and then applying laser energy to the lens implant in the pocket using a laser to correct refractive errors of the lens implant and/or the eye in a non-invasive manner. As in the first illustrative embodiment of the intracorneal lens implantation procedure explained above, no flap is formed in the cornea of the eye. Also, the front surface of the cornea is not ablated using a PRK procedure.

Initially, in the second illustrative embodiment of the intracorneal lens implantation procedure, a lens implant is soaked in a cross-linking solution held in a container prior to its insertion into a corneal pocket in the eye so that the lens implant is pre-coated with the cross-linking solution thereon. The lens implant has a predetermined shape for changing the refractive properties of an eye, and is flexible and porous so that fluids (e.g., oxygen, electrolytes, glucose, etc.) are able to freely pass through the lens implant. In the second illustrative embodiment, the lens implant may comprise a hybrid lens implant as described above with regard to the first illustrative embodiment, or may comprise any of the other characteristics described above with regard to the lens implant 308. The coated surface of the hybrid lens implant may be organic and hydrophilic, and may formed using a desired thickness that can be cross-linked with UV light and riboflavin before or after its implantation. Also, in the second illustrative embodiment, the cross-linking solution may comprise a photosensitizer in the form of riboflavin, and/or a liquid suspension having nanoparticles of riboflavin disposed therein. Preferably, the cross-linker has between about 0.1% riboflavin to about 100% riboflavin therein (or between 0.1% and 100% riboflavin therein).

Next, in the second illustrative embodiment of the intracorneal lens implantation procedure, a pocket is formed in the cornea of the eye. The formation of the corneal pocket in the cornea of the eye allows one to gain access to the tissue bounding the pocket (i.e., the interior stromal tissue bounding the pocket). In particular, in the second illustrative embodiment, the pocket is formed by making an intrastromal incision in the cornea of the eye either by using a femtosecond laser (i.e., the incision is cut in the cornea using the laser beam(s) emitted from the femtosecond laser) or by using a mechanical keratome (e.g., a mechanical microkeratome).

After the pocket is formed in the cornea of the eye, the lens implant with the photosensitizer provided thereon (e.g., riboflavin) is inserted inside the pocket so that the photosensitizer permeates at least a portion of the tissue bounding the pocket. In particular, in the illustrated embodiment, the lens implant is inserted into the corneal pocket through a very small incision using a pair of forceps or microforceps. The photosensitizer facilitates the cross-linking of the portion of the tissue bounding the pocket.

Then, shortly after the lens implant with the photosensitizer is inserted inside the pocket, the cornea of the eye is irradiated from the outside using ultraviolet (UV) radiation so as to activate cross-linkers in the portion of the tissue bounding the pocket, and thereby stiffen the cornea, prevent corneal ectasia of the cornea, and kill cells in the portion of the tissue bounding the pocket. In the illustrative embodiment, the ultraviolet light used to irradiate the cornea may have a wavelength between about 370 nanometers and about 380 nanometers (or between 370 nanometers and 380 nanometers). Also, in the illustrative embodiment, only a predetermined anterior stromal portion of the cornea to which the photosensitizer was applied from the lens implant is cross-linked (e.g., only the bounding wall of the corneal pocket), thereby leaving an anterior portion of the cornea and a posterior stromal portion of the cornea uncross-linked. That is, in the illustrative embodiment, the entire corneal area inside the cornea exposed to the cross-linker is selectively cross-linked, thereby leaving the anterior part of the cornea and the posterior part of the stroma uncross-linked. The portion of the cornea without the cross-linker is not cross-linked when exposed to the UV radiation. In an alternative embodiment, the cornea may be irradiated using microwaves as an alternative to, or in addition to being irradiated using ultraviolet (UV) radiation.

In the second illustrative embodiment of the intracorneal lens implantation procedure, the irradiation of the cornea using the ultraviolet (UV) radiation only activates cross-linkers in the portion of the stromal tissue bounding the pocket, and only kills the cells in the portion of the tissue bounding the pocket, so as to leave only a thin layer of cross-linked collagen to prevent rejection of the lens implant and/or encapsulation by fibrocytes, while preventing post-operative dry eye formation. In addition to preventing encapsulation of the lens implant by fibrocytes, the cross-linking of the stromal tissue bounding the pocket also advantageously prevents corneal haze formation around the lens implant. That is, the cross-linking of the stromal tissue surrounding the lens implant prevents formation of myofibroblast from surrounding keratocytes, which then convert gradually to fibrocytes that appear as a haze, and then white encapsulation inside the cornea, thereby causing light scattering in front of the patient's eye.

After the lens implant has been inserted into the pocket in the cornea of the eye, laser energy is applied to the lens implant in the pocket using a laser so as to correct refractive errors of the lens implant and/or the eye in a non-invasive manner. In the second illustrative embodiment, a two-photon or multi-photon laser is used to apply the laser energy to the lens implant in the pocket so as to modify the index of refraction of a discrete internal part of the lens implant in a non-invasive manner, while preventing post-operative dry eye formation. In the second illustrative embodiment, the laser energy applied by the two-photon or multi-photon laser has a predetermined energy level below an optical breakdown power level of the two-photon or multi-photon laser.

As described above with regard to the first illustrative embodiment of the intracorneal lens implantation procedure, prior to the application of the laser energy to the lens implant in the pocket by the two-photon or multi-photon laser, a virtual model of the lens implant may be generated, and the two-photon or multi-photon laser may be focused in accordance with the virtual model. In particular, a specially programmed data processing device (i.e., a specially programmed computing device or computer) is used to generate a virtual model of the lens implant so that a new index of refraction of the lens implant at the focal point of the two-photon or multi-photon laser is capable of being determined prior to the application of the two-photon or multi-photon laser. Then, the specially programmed data processing device (i.e., a specially programmed computing device or computer) is used to focus the two-photon or multi-photon laser non-invasively outside the eye in accordance with the virtual model generated for the lens implant.

In a third illustrative embodiment of the intracorneal lens implantation procedure with the cross-linking of the cornea, the procedure may be performed in a similar manner to that described above with regard to the second illustrative embodiment, except that the laser energy may be applied to the lens implant in the pocket by the laser prior to the irradiation of the cornea, rather than after the irradiation of the cornea as described above in the second embodiment.

In further illustrative embodiments, synthetic lenslets are created from collagen, which is modified in the process of lenslet production and subsequently after implantation, to prevent rejection of these lenslets by the host tissue. In his previous published patents (see e.g., U.S. Pat. Nos. 9,937,033, 10,278,920, 10,314,690, and 10,583,221, which are hereby incorporated by reference as if set forth in their entirety herein), the present inventor described that crosslinking the human corneal implant eliminates the human corneal immune response to the human crosslinked cornea and crosslinking the wall of the host cavity practically eliminates the host's tendency to induce an immune reaction against the implanted tissue. As described in these patents, the host's cells surrounding a corneal cavity are killed by crosslinking and the host corneal collagen in that area is also crosslinked. The crosslinking changes the molecular structure and bounding of the amino acids, peptides, and proteins creating many crosslinked bonds that make the tissue more resilient, while maintaining the transparency of the lenslet and eliminating their immunogenicity since they do not have free molecular attachments. Therefore, practically, one creates an immune privileged space inside the host cornea.

In one embodiment, the synthetic lenslet will have a compensatory refractive surface that modifies the refractive error of the host after its implantation, that is myopia, hyperopia astigmatism, and presbyopia by using either an excimer laser or a femtosecond laser with a Shack-Hartmann sensor and wavefront technology to modify the surface of the lenslet prior to its implantation in the host corneal cavity. In addition, this procedure adds to mechanical stability of the cornea because it is crosslinked.

In one embodiment, crosslinking of the synthetic inlay can be done by one or a combination of crosslinkers, such as riboflavin, xanthine, derivatives Rose Bengal, erythrosin, eosin, and phthalocyanine, porphyrin hypericin, and Rose Bengal and mixtures thereof, or other synthetic dyes, such as porphyrins, 5-aminolevulinic acid, polymeric photosensitizers, using an appropriate wavelength of a laser light.

In one embodiment, the corneal synthetic implant is produced by 3-D printing technology or molding of organic collagen material or a combination collagen with other polymers of group of chitosan, elastin, hyaluronic acid, having an index of refraction of 1.3 building a refractive lenslet of a predetermined shape that corrects the refractive power of an eye having either myopic, hyperopic, astigmatic, or presbyopia or a combination thereof, wherein the synthetic lenslet is implanted in a preformed corneal pocket created with a femtosecond laser where the synthetic lenslet along with a photosensitizer is injected inside the stromal pocket followed by crosslinking the implant and the wall of the corneal stroma by ultraviolet (UV) radiation to prevent rejection of the implant and provide resiliency to the cornea and correct refractive error of the eye.

In one embodiment, a slurry fluid containing collagen at a concentration of 1%-98% w/w or 10%-30% or 15% w/w to 50% w/w or 50% to 80% w/w or combined with other polymers, such chitosan or elastin or hyaluronic acid, etc. and a photosensitizer, such as riboflavin or Rose Bengal at concentration of 0.1%-1% injected in a corneal pocket created with a femtosecond laser wherein the synthetic collagen can correct the hyperopic refraction or presbyopia under control of a Shack-Hartmann sensor along with a photosensitizer injected inside the stromal pocket followed by crosslinking the implant and the wall of the corneal stroma by UV radiation using a UV laser at power of 3 mW/cm2 to 20 mW/cm2 for a short period or time of 1 minute to 10 minutes depending on the power of the UV laser and the concentration of the riboflavin to solidify the gel to correct hyperopia or presbyopia under the control of a Shack-Hartmann sensor and to prevent rejection of the crosslinked collagen and provide resiliency to the cornea.

In one embodiment, the corneal synthetic lenslet is produced by injection printing technology under control of a Shack-Hartmann sensor and optical coherence tomography (OCT), having a composition of organic collagen type I or a combination collagen 15% to 30% w/w or along with other polymers of group of chitosan, elastin, hyaluronic acid of less than 3% w/w and riboflavin concentration of 0.1-3% w/w or 1% w/w to 5% w/w or more is injected inside a 3-D printer unit to create a synthetic refractive lenslet which is crosslinked partially with UV radiation and injected in a corneal pocket with riboflavin and crosslinked the synthetic lenslet and the wall of the corneal stroma with the UV radiation, wherein the synthetic lenslet corrects the hyperopic, myopia, or astigmatic refraction or presbyopia and OCT.

In one embodiment, synthetic implant is made of a collagen material having a concentration of 15% w/w and other polymeric compounds are less than 1% w/w to 5% w/w or more.

In one embodiment, the synthetic lenslet contains collagen and polyethylene glycol stabilized chitosan in addition to collagen type I with some Type III collagen.

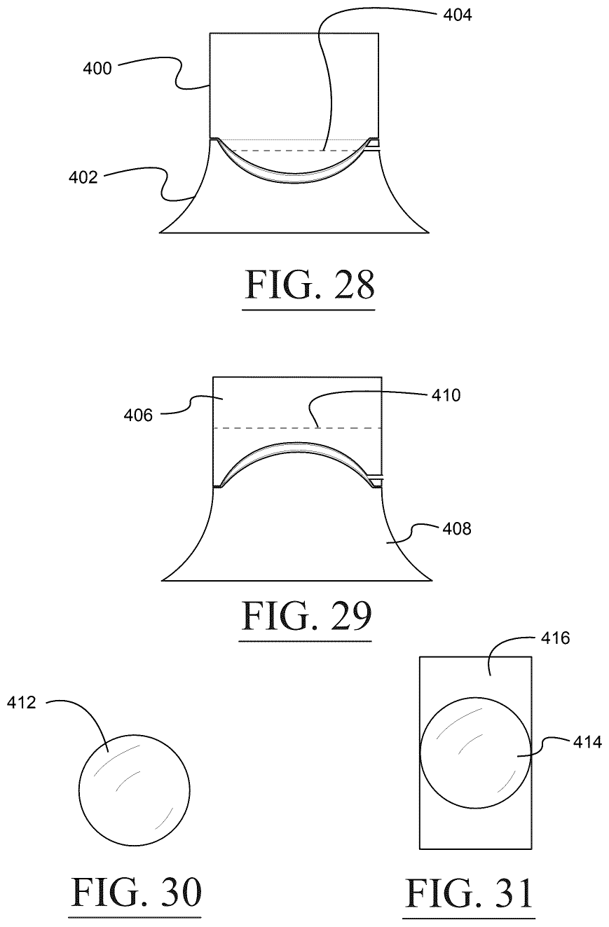

In one embodiment, the container or the mold has a predetermined surface that produces either a convex or concave or an astigmatic lenslet (see FIGS. 28-31, 34C, 34D, 35A, 35B, 36A, and 36B) in any outer shape (e.g., circular, rectangular, or square etc.). For example, a circular synthetic lenslet 412 is depicted in FIG. 30, while a circular lenslet optic 414 with a rectangular peripheral edge 416 is depicted in FIG. 31. In FIG. 28, a concave synthetic lenslet is formed from collagen 404 by using an upper mold portion 400 and a lower base mold portion 402. In FIG. 29, a convex synthetic lenslet is formed from collagen 410 by using an upper mold portion 406 and a lower base mold portion 408.

In one embodiment, the mold is from 2-14 millimeters (mm) in diameter or more.

In one embodiment, the lenslet can have a thickness of 50 microns to 2 mm or more.

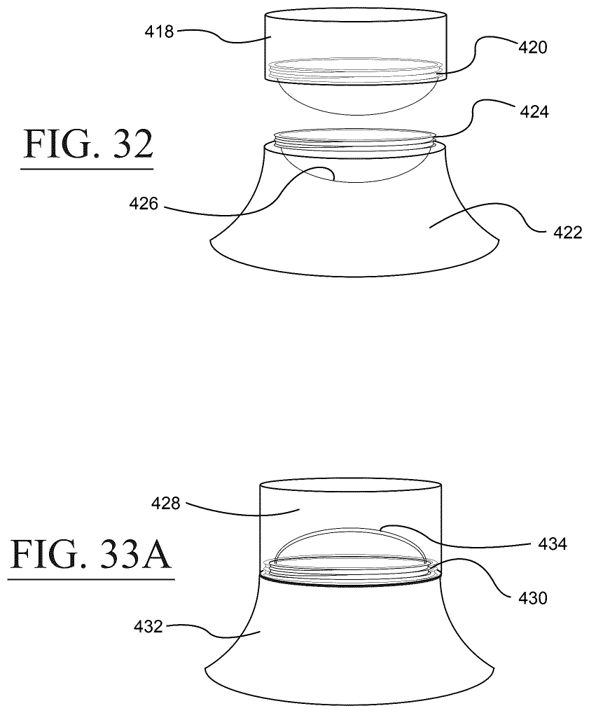

In one embodiment, the container has a surrounding lip which has a vertical, 90 degree or 45 degree or more tilt to the surface of the mold and extends beyond the bottom surface of the mold to create a circular stable support in which the mold is separated from the horizontally placed smooth surface such as glass (see FIGS. 32 and 33A). For example, the bases 402, 408, 422, 432, 440 of the molds depicted in FIGS. 28, 29, 32, 33A and 33B are all flared radially outward at the bottoms thereof to add more stability to the molds.

In one embodiment, the vertical lip fits in a groove at the edge of the surface of the horizontal back plate (see FIG. 32) that can be rotated in or out thereby releasing the lenslet from the lip. In FIG. 32, the base 422 of the mold has concave surface 426 for forming a concave synthetic lenslet. The upper portion 418 of the mold is threadingly engaged to the base 422 of the mold by means of the upper internal threads 420 on the upper portion 418 engaging with the corresponding external threads 424 on the base 422 of the mold. In FIG. 33A, the base 432 of the mold has convex surface 430 for forming a convex synthetic lenslet. The upper portion 428 of the mold is threadingly engaged to the base 432 of the mold by means of the upper and lower internal threads 430.

In one embodiment, the plate and the lip can be screwed in or out or in another method known in the art, releasing the formed lenslet after it is crosslinked.

In one embodiment, the corneal synthetic implant is produced by 3-D printing technology using collagen type 1 or in combination with type III collagen with or without other polymers, such as chitosan, elastin, hyaluronic acid, having an index of refraction of 1.3 and having short acting photosensitizer carbodiimide and riboflavin is used in building a lenslet of predetermined shape and/or refractive power where the carbodiimide initiate a slight crosslinking without a complete crosslinking, while the addition of riboflavin is activated after 3-D printing with UV radiation to provide a more permanent crosslinking of the lenslet that corrects the refractive power of an eye having a refractive error of myopic, hyperoptic, astigmatic, or presbyopia or a combination thereof, when implanted in a corneal pocket through a small incision made with a femtosecond laser of 1-2 mm in diameter along with 0.1% or more riboflavin solution which crosslinks the implant and the wall of the corneal stroma by UV radiation and prevents rejection of the implant and dry eye formation in the post-operative period by protecting majority of the sub-bowman nerve plexus.

In one embodiment, the two stage crosslinking permits the moderately crosslinked collaged scaffold to pass through the nozzle of the 3-D printer while the activation of the riboflavin with the UV laser subsequently completes the crosslinking of the lenslet and the wall of the corneal pocket to prevent future rejection.

In one embodiment, the lip 438 of the mold has micro holes 439 therein that permits a part of the fluid to escape if a plunger 436 (see FIG. 33B) is pressed on the synthetic collagen 442 (in the down direction of arrow 444) so that the thickness of the lenslets be reduced less than 50%-75% or less of its original volume, while the temperature of the mold is gradually increased to 37 degree C. body temperature and simultaneously the lenslet composite is irradiated with UV laser radiation of 300-420 nm wave length, preferably 375-380 nm wavelength, for a period of a few minutes which convert the liquid collagen to a collagenous resilient scaffold to make the ultimate shape of the lens stable after its implantation in a corneal pocket with further UV radiation of the lenslet and the wall of the corneal pocket to prevent its rejection by killing the corneal cells in the wall of the corneal pocket around the synthetic collagen lenslet.

In one embodiment, the shape of the collagen does not change by the crosslinking but converts a part of the collagen to crosslinked collagen scaffold and increases its biomechanical stability.

In one embodiment, the synthetic lenslet is made of mostly collagen type I alone or with some type III collagen as found in the cornea and crosslinked with a photosensitizer, such as riboflavin or Rose Bengal or other crosslinkers and UV radiation. In one embodiment, the lenslet will have a final thickness of 250 microns or less.

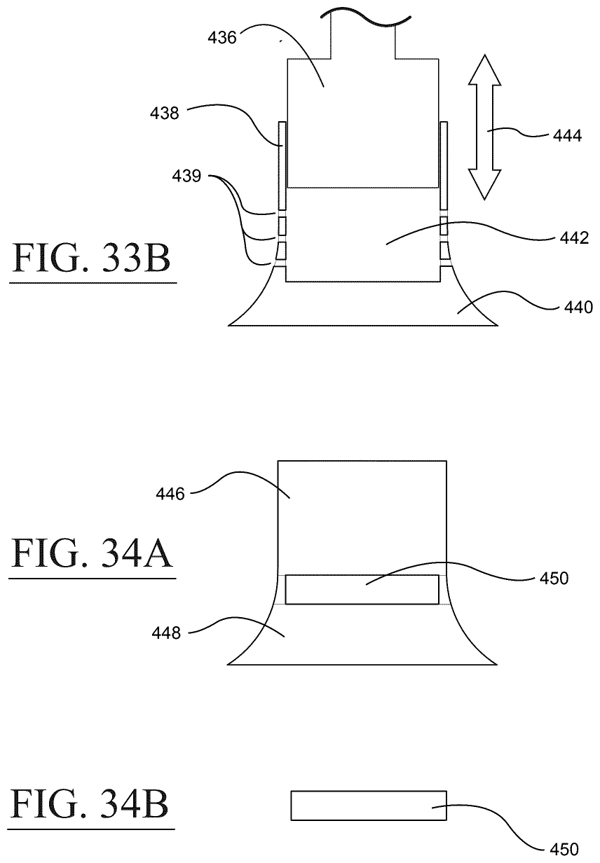

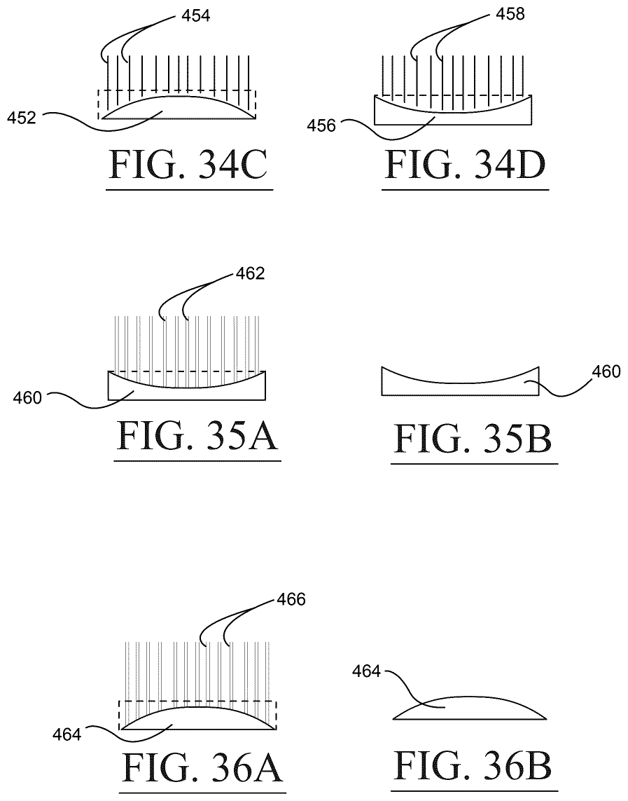

In one embodiment, the lenslets 452, 456 are created from +0.1 to 20.00 dioptric power, or alternatively from −0.1 to 20.00 dioptric power, or the lenslet is irradiated with an excimer laser 454, 458 (see FIGS. 34C and 34D) to create a surface from −0.1 to 20.00 dioptric power or an astigmatic refraction of −0.1 to −5.00 D or 0.1 to 5.00 D or in combination.

In one embodiment, with reference to FIGS. 34A-34D, the mold with lip 446 and base 448 is created with a parallel synthetic organic lenslet 450 while a combination of spherical or astigmatic power can be formed with an excimer laser and a Shack-Hartmann sensor to the exact dioptric power for the correction of each person's refractive error in the operating room prior to the implantation in a prepared corneal pocket that has been prepared with a femtosecond laser or mechanically.

In one embodiment, the front surface of the synthetic implant is flat and the posterior surface is convex or concave as shaped by the mold, but the lenslet is implanted with its refractive surface facing out toward the corneal epithelium and the flat surface of the lenslet lies on the posterior corneal surface of the corneal pocket.

In another embodiment, the synthetic collagen lenslet 460, 464 has a parallel surface which can be shaped to any surface curvature (e.g., concave or convex) by cutting away a part of the lens surface with a femtosecond laser 462, 466 (see FIGS. 35A, 35B, 36A, and 36B) under an OCT observation and a software to the desired lenslet's surface or refractive power to correct the refractive power of an eye, where a corneal pocket is created with a femtosecond laser in the cornea, the synthetic lenslet is soaked in a solution of riboflavin, and a small incision is made with a femtosecond laser in the corneal pocket, followed by implantation of the lenslet and injection of riboflavin in the corneal pocket and subsequent UV irradiation to completely crosslink the lenslet and its surrounding wall of the corneal stromal pocket.

In another embodiment, the solution of collagen alone or with another polymer is mixed with a photosensitizer, such as riboflavin or Rose Bengal, and is poured in a 3-D printer, and heated to 37 degrees C. and irradiated with UV radiation of 3 mW/square for 1-2 minutes to create some scaffold collagen, then the 3-D printer unit is activated with appropriate software to print out in 3-D the synthetic collagen/polymer combinations to a desired shape and refractive power with an index or the refraction of 1.3, the lenslet is implanted in the corneal stroma after soaking it in the operating room, and creating a corneal pocket injected with riboflavin and hyaluronic acid for ease of the implantation, after which the cornea is UV radiated.

In one embodiment, the synthetic lenslet is placed in an injector having a needle and the lenslet combined with hyaluronic acid as a lubricant and riboflavin simultaneously are injected inside the prepared corneal pocket followed with UV radiation to crosslink the lenslet and the wall of the corneal pocket and prevent an immune response to the lenslet.

In one embodiment, the lenslet carries slow release polymeric nanoparticles which have medication(s) such as anti-inflammatory and antibacterial medications, etc.

Since the water content of the normal cornea is about 78-80% of the cornea and the collagen component is about 15-20%, the composite of the synthetic collagen hydrogel corneal lenslet can be made in the similar range of water concentration or less, e.g., 50% to 10% or less without changing the refractive index of the cornea by crosslinking it while increasing the density of the fibrils in the lenslet.

In one embodiment, the collagen used can be Type I, Type II, Type III, Type IV, Type V, Type VI, Type XI collagen, or a combination thereof, but preferably Collagen type I.

In one embodiment, the molded lenslet is removed from the mold by removing the part around the lenslet or immersing the mold in the phosphate buffered solution containing riboflavin that penetrated the lenslet and the lenslet and the wall of the corneal cavity are crosslinked subsequently with UV radiation that crosslinks the lenslet, the wall of the corneal cavity and eliminate potential infective germs by photodynamic therapy.

In one embodiment, the composition of the collagen hydrogel, e.g., type I collagen is generated by mixing collagen powder with a desired concentrations mixture with only one crosslinker, such as riboflavin in 0.1% to 2%, etc. that does not crosslink the collagen molecules without presence of the UV light including UVA and UVB or UVC; other non-photoactivated chemical crosslinker starting crosslinking immediately after their addition to the collagen hydrogel are not as well controlled as the photoactivated ones, wherein the composite is heated to 37 degrees C. to initiate a phase transition and some febrile mesh from in the gel, then the fluid is expressed out of the mixture mechanically to the desired thickness by compressing the collagen hydrogel in the container, then exposing the hydrogel lenslet plus crosslinked or photosensitizer with UV radiation or other wavelength of light depending on the photosensitizer to achieve a lenslet texture for its refined surface modification with an excimer laser or femtosecond laser and implantation in the cornea pocket that is crosslinked with riboflavin and UV radiation.

In one embodiment, for molding, the collagen and water plus riboflavin are mixed, then compressed to reach a thickness of about ½-⅓ of its original thickness or less and crosslinked with one crosslinker, such as riboflavin/UV laser light with a power of 15-30 mW/cm2. Since the surface of the lenslet defines the refractive correction needed not its index of the refraction, the lenslet is crosslinked, then shaped by modifying its already refractive surface with an excimer laser or cut with a femtosecond laser to the desired refractive power as needed to compensate for the patient's refractive errors, using a wavefront technology and Shack-Hartmann sensor prior to its implantation.

In one embodiment, the bottom surface of the mold where the collagen gel is formed defines if the lenslet will be a convex lenslet or concave lenslet or an astigmatic lenslet, etc.

In one embodiment, the photosensitizer can be xanthine, or any other photosensitizer that generates a photodynamic effect producing singlet oxygen and reactive species that initiate crosslinking of the proteins, such as collagen when exposed to a laser light that is absorbed by the photosensitizer.

In one embodiment, the density of the lenslet collagen hydrogel mesh is increased to prevent cell migration in the lenslet by removing >95% of its water content, then further cross-linking and its surface is shaped to create a lens let that is difficult to be invaded by inflammatory cells, etc. and the corneal pocket's wall is crosslinked after lenslet implantation to create an amorphous crosslinked lenslet with a crosslinked wall of the host corneal cavity which now will not induce an immune response in the host cornea or its erosion.

In one embodiment, the lenslets are combined with polymeric slow drug release nanoparticles such as polylactic, polyglycolic acid, or a mixture thereof or polycaprolactone, polyanhydrides, micelles etc. that can penetrate the lenslet and release medication after their implantation as described in U.S. Pat. No. 10,278,920 describing a corneal drug delivery system, which is hereby incorporated by reference as if set forth in its entirety herein.

In one embodiment, the concentration of the collagen gel, e.g., 1-15% and water of 50 to 70%/riboflavin mixture or 0.1%-2% and the temperature of the medium 37 degrees C. defines how slurry the mixture of collagen gel/fibrils is to pass through the nozzle of the 3-D printer to make a 3-D lenslet to the desired width of 2-14 mm and thickness of 0.1-1 mm, and curvature of its surface, convex or concave or astigmatic, and the mechanical stability of the lenslet is defined by the degree of its crosslinking with the UV radiation of 10 mW/cm2 to 30 mW/cm2 or more after the initial lenslet is formed by the 3-D printer.

In one embodiment of the 3-D printed lenslet, the polymeric slow release nanoparticles of polylactic or polyglycolic acid, etc. are added to the collagen gel before it is pressed out of the nozzle of the 3-D printer unit, to release the medication after implantation in the corneal pocket which is crosslinked simultaneously with a photosensitizer along with implantation of the lenslet, which is crosslinked with UV radiation.

In one embodiment of the molded lenslet, the mold container is made of two sections: (a) a hollow cylindrical upper portion and (b) a base portion with a curved smooth surface that combine to make a container where the surface of the base can be convex, concave, astigmatic, or flat. In the mold, collagen type I can be used, or collagen type I in combination with collagen type II or III, and/or recombinant collagen can be used. The percentage of the collagen in a physiological solution can be from 1% to 25%, and water to which riboflavin is added at desired 0.1%-2% concentrations are pored inside the mold container, the collagen swells in the water and expands without being dissolved, thus building a collagen mesh that is not crosslinked. One can add any medications desired in a non-toxic concentration to be released as slow release polymeric nanoparticles, to the solution at this stage, so as to prevent infection or inflammation, etc.

In one embodiment, by increasing the temperature of the mold and the collagen hydrogel/riboflavin solution to 37 degrees C., small fibrils are formed inside the mold. This mixture can be used in developing a lenslet by molding or using a 3-D printer by exposing the collagen mesh to low power UV radiation such as 1-3 mW/cm2 for 1-2 minutes, the fibrils are slightly more interconnected, but they are not resilient and can be manipulated to pass through either through the nozzle of the 3-D printer or they can be used for molding.

In one embodiment of the molded lenslet, the excessive water content of the mold can be removed by using a software controlled plunger 436 that fits inside the mold and pushes the gel 442 to a desired degree forcing the fluid in the collagen hydrogel to exit through the small side holes in the wall of the mold (see FIG. 33B), thus condensing the collagen fibrils and non-crosslinked mesh to a smaller space as needed thereby reducing gradually the water content of the gel that is not completely crosslinked, but can be manipulated to become thinner to the desired thickness. The combination of increased temperature and pressure applied to the hydrogel mixture with controlled crosslinking using various amount of the UV power defines the precise degree of thickness and density or crosslinked collaged that ultimately determines its resiliency.

In one embodiment of the 3-D printed lenslet, the mixture of hydrogel at 37 degrees C. and partially crosslinked hydrogel is compressed to the degree that it can pass through the 3-D printer's nozzle to form the lenslet under the control of the printer's software and build a lenslet this is convex, concave, astigmatic, or any combination thereof and to the desired shape. At this stage, the shape or refractive power of the molded lenslet or 3-D printed lenslet can be further modified with the use of a femtosecond laser or an excimer laser under the control of software using wavefront technology and a Shack-Hartmann system to create a lenslet surface to the desired convexity, concavity, or astigmatism to correct precisely the refractive power of the patient as needed.

In one embodiment, a corneal stromal cavity in the patient's corneal stroma is prepared to the desired size so that the lenslet can be implanted in it with ease or with the help of an injector and viscoelastic material, such as heparin through a small incision made to access the cavity in the peripheral cornea.

In one embodiment, a small amount (0.05 ml) of riboflavin or other photosensitizer is injected in the stromal cavity over the lenslet to penetrate the lenslet and the wall of the cavity.

In one embodiment, ultraviolet (UV) radiation is applied at desired power of 3-30 mW/cm2 or more and a desired time of 1-5 minutes through the corneal surface to further crosslink the lenslet and the wall of the stromal cavity preventing an immune response from the host.

In a further embodiment, one uses a human cornea or cornea from genetically modified animal, such as a pig or collagen obtained mainly from a cornea (type III collagen) or a mixture of various collagen types used as inlay for creation of a supporting tissue, or modification for refractive errors or as pinhole and crosslinked because, the crosslinked corneal tissue does not inhibit transport of the water and nutrients through it regardless where in the corneal stroma it is implanted, the water and nutrients pass from the back side of cornea through the inlay (implant) to the other side of the cornea or water etc. passes from the front to the back of the cornea. As a result, the crosslinked corneal inlay does not create tissue anoxia or foreign body immune response, etc. to lead to cellular immune stimulation that would cause rejection of the inlay as seen with previous polymeric, e.g., acrylic solid lenses, when implanted inside the cornea.

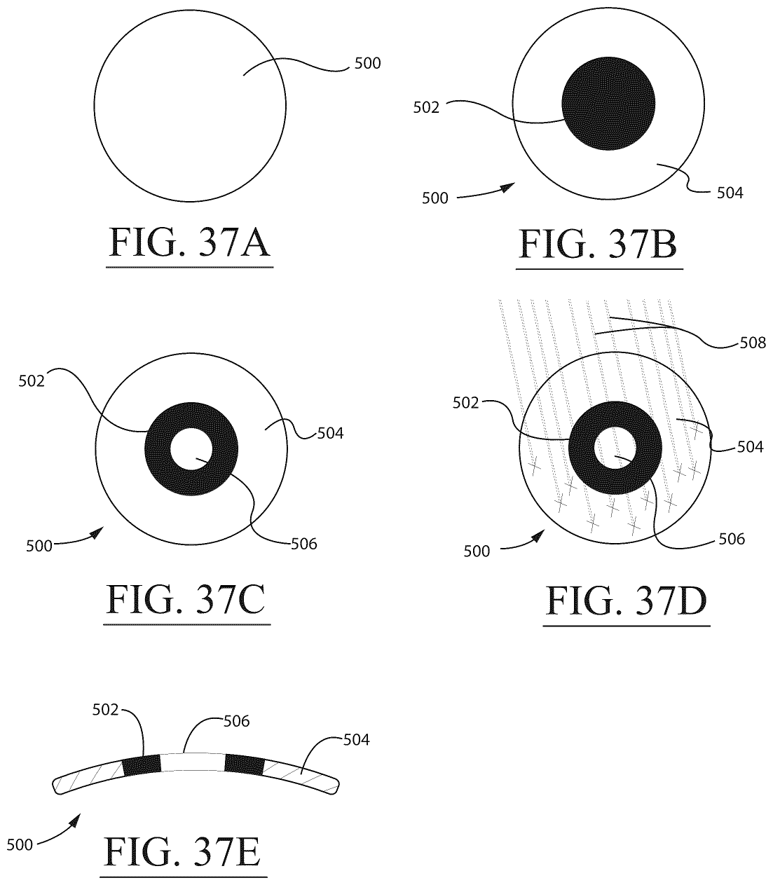

In one embodiment, the corneal inlay of 5-9 mm in diameter and a thickness of 10-300 microns is obtained from human eye bank eye, or genetically modified cornea of an animal, or 3D printed corneal collagen is tattooed at its center with a biocompatible non-toxic dark or black India ink or acrylic black ink, etc., the central tattooed area 502 of the inlay 500 encompasses an area of 1 mm-5 mm (see FIGS. 37A-37C) of the inlay, and is placed in a solution of riboflavin at a concentration of 0.1-1% with or without methylene blue at concentration of 1 microgram/ml for 10 minutes or more to damage the RNA or DNA of the cells or any contaminated organism, viral, fungal, bacteria or parasites, etc. that might be present in the human or animal corneal inlay, then using a trephine or a femtosecond laser, etc., a central inlay tissue of 1-3 mm, preferably <2 mm, is cut and removed, (FIGS. 37B and 37C) leaving a rim of tattooed tissue of at least 1-1.5 mm or more having a dark tattooed area 502 around the through and through hole 506, then the inlay is crosslinked with a UV laser light 508 at a wavelength of about 320-380 nm and power of 3 mW/cm2-10 mW/cm2 for a period of 2-10 minutes or more to crosslink (see FIG. 37D) the proteins, glycoproteins, and the RNA or DNA of the cells in the inlay are crosslinked making the inlays less antigenic or not immunogenic for the host cornea, and killing all pathogenic organism that might be present in it, then the crosslinked inlay with its central hole 506 and dark rim 502 having a clear remaining peripheral area 504 (FIG. 37C) can be kept in a physiologic solution or sterile albumin etc. and or may be irradiated with gamma radiation and stored for a year or more as needed in a sterile container.

In one embodiment, a corneal inlay with a central hole is made from a human or a genetically modified animal cornea, such as a pig. A central hole is cut in the inlay, the inlay is cross-linked with a photosensitizer, such as riboflavin and or methylene blue, and irradiated with UV radiation. A darkened ring has a central hole that is made with a diameter of 2-4 mm formed from flat darkened stainless steel or any other polymeric organic, laser ablatable or synthetic non-magnetic material with a thickness of 10-50 microns or more to which tissue adhesive is applied to its outer border and placed in the center of the inlay and irradiated with a laser to create a tight adhesion to the inlay.

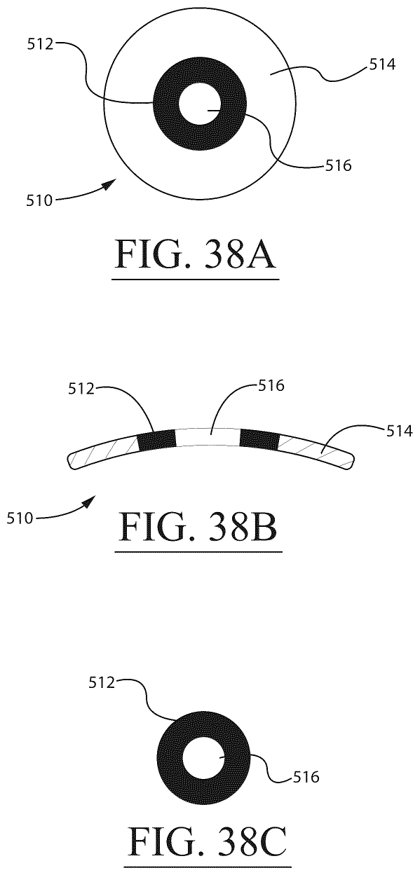

In one embodiment, a corneal inlay is made from human eye bank eye, or genetically modified cornea of pig or another animal, or 3-D printed collagen or molded from collagen and other proteins, then the central 4-5 mm area of the inlay will be tattooed with a biocompatible non-toxic dark or black India ink or acrylic black ink, or a combination of polyvinylidene fluoride and carbon nanoparticles, etc., a hole with a diameter of <2 mm (see FIGS. 37B-37C and FIGS. 38A-38B) is cut away from its center, then the inlay is crosslinked with a photosensitizer, such as riboflavin, and irradiated with UV radiation for use as an inlay where its surface can be modified with an excimer laser after its placement over the corneal stroma under a LASIK flap to correct the refractive power of the eye for far and simultaneously create a pinhole effect in the center of the cornea to extend the focal point of the cornea for near vision in presbyopia or even in younger patients with myopia, hyperopic, with or without astigmatism or keratoconus cornea to see in focus the near objects and strengthen the mechanical stability of the cornea.

In one embodiment, as described the pinhole is created inside a corneal inlay with >5 mm in diameter then a central 4 mm tattooed area is cut with a trephine of femtosecond laser creating a hole 506 with a tattooed rim of <2 mm in diameter or more (see FIGS. 37B-37C) this organic pinhole can be implanted in the center of the cornea inside a cavity produced with a femtosecond laser using an injector to deposit the inlay in the center of the eye cavity correcting the presbyopia in older patients, the inlay and the cavity is crosslinked with riboflavin injected in the corneal cavity and irradiated with UV radiation to crosslink the inlay and the tissue around it preventing rejection of the pinhole.

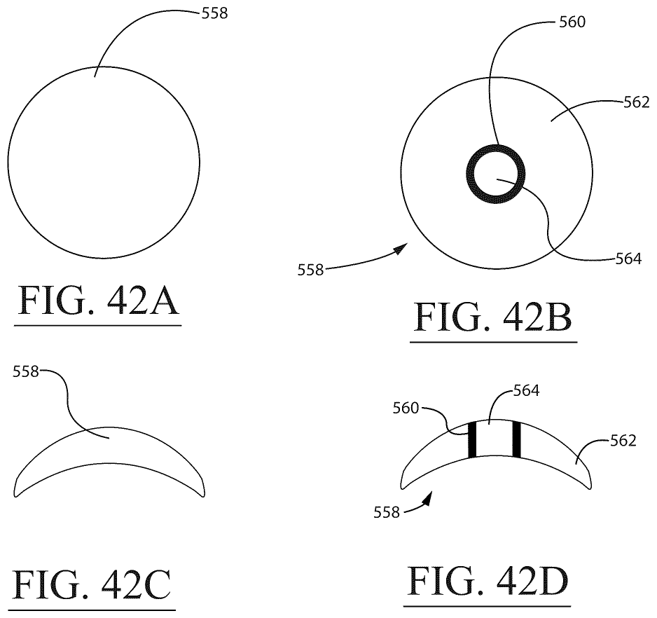

In one embodiment, a virtual pinhole with the diameter of 3-4 mm is created by tattooing the peripheral 2 mm diameter of the corneal inlay 558, having a thickness of 10-30 micron creating a virtual pinhole 564 (see FIGS. 42B and 42D) using a biocompatible non-toxic dark or black India ink or acrylic black ink or carbon nanoparticles etc. creating a virtual central virtual pinhole of 1-2 mm with a darkened wall and a clear peripheral area 562, then the inlay 558 is crosslinked with riboflavin with or without methylene blue combination and UV radiation, implanted in the host corneal stroma of a presbyopia eye after making a pocket in the corneal stroma with a femtosecond laser unilaterally or bilaterally that does not need to be corrected for its refractive error, where the corneal pinhole inlay provides an extended focal point for the eye permitting the person to read near and also see far objects in focus through the pinhole and the corneal pocket are simultaneously crosslinked with riboflavin and UV radiation to prevent rejection of the inlay having a virtual hole in the center of it since the center part of the inlay is not tattooed.

In one embodiment, a pinhole inlay having a diameter of 3-5 mm with a thickness of 10-20 microns is formed using a femtosecond laser, and the inlay is entirely tattooed with a biocompatible non-toxic dark or black India ink or acrylic black ink, or a combination of polyvinylidene fluoride and carbon nanoparticles, etc., then the inlay is crosslinked with riboflavin and/or methylene blue combination and UV radiation, and using a trephine or a femtosecond laser, a central hole of 1-2 mm is cut in the tattooed inlay, the pinhole dark corneal inlay is implanted inside the central part of the host cornea using an injector and hyaluronic acid after creating a 5 mm in diameter pocket in the center of the host cornea to treat presbyopia eliminating the potential rejection of the pinhole inlay.

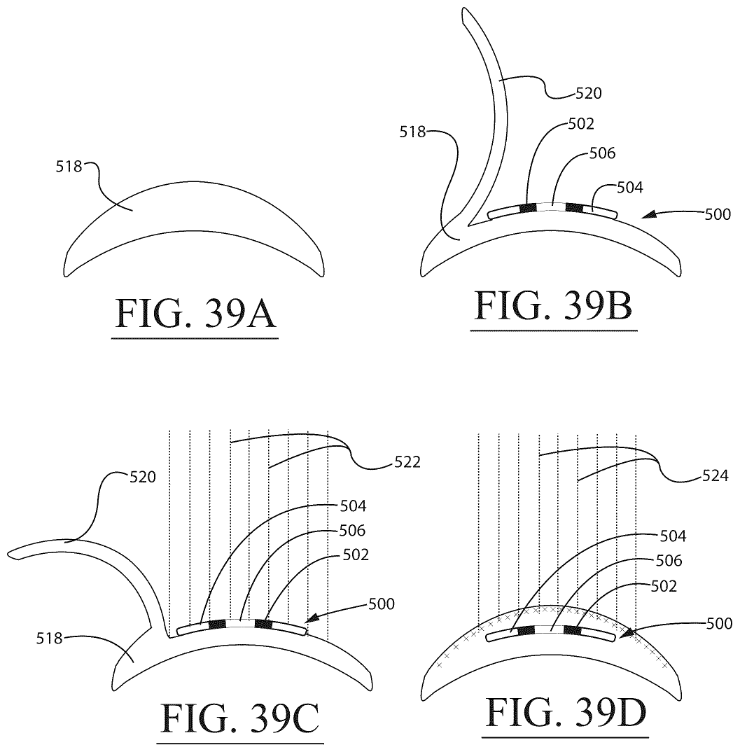

In one embodiment, the inlay 500 from human eye bank eye, or genetically modified cornea, or 3-D printed collagen or molded from collagen and other proteins with the central virtual or a regular hole 506 and darkened tattooed rim 502 is prepared for implantation in the human cornea using a LASIK flap 520 in the host cornea 518 (see FIGS. 39A-39B). Initially, the visual axis of the eye is located on the corneal surface is marked with a pen, then using a femtosecond laser a 300 degree corneal flap 520 is prepared and turned to the side to expose the corneal stroma (see FIGS. 39A-39B), the location of the visual axis is marked with a pen on the corneal stroma and the inlay 500 is placed so that the center of the inlay's virtual/or hole coincides with the visual axis, then using an excimer laser 522 the surface of the inlay is ablated under control of wavefront technology to correct refractive error of the entire eye (see FIG. 39C), then a few drops of riboflavin is placed over the surface of the inlay 500 and the LASIK flap 520 is repositioned back over the inlay 500 to absorb the riboflavin. The cornea 518 is then irradiated with UV light 524 to crosslink the tissue surrounding the inlay and prevent its rejection (see FIG. 39D). The corneal crosslinking can be repeated with external application of riboflavin and UV light in the post-operative period to stabilize the inlay and prevent its rejection, these can be combined with topical application of any one of anti-inflammatory agents or antibiotics known to the experts. In one embodiment of synthetic cornea, there may not be need for the crosslinking step.

In one embodiment, the corneal inlay with a central hole or virtual hole is 3-D printed or molded as described above to which a darkened polymeric ring 512 (see FIGS. 38A-38C) with a biocompatible non-toxic dark or black India ink or acrylic black ink etc. is produced inside the collagen molecules which will have a transparent peripheral part inlay rim 514 while an acrylic India ink etc. is injected with collagen in process of 3-D printing using the computer to form a 2-4 mm flat darkened ring 512 in the center of the inlay 510 with a transparent central part 516 including polymeric organic material with a thickness of 10-100 micron or more, while the central opening 516 or virtual aperture in the ring is between 1-2 mm and having a rim of 1-2 mm dark ring 512 surrounded by the central optically transparent collagen and outer transparent part 514 with a diameter of 5-9 mm to which riboflavin at a concentration of 0.1-1% is added after the inlay 510 is formed, in order to crosslink the inlay 510 with UV radiation prior to the implantation and kill potential pathogenic organisms and making the inlay 510 less or not immunogenic. The inlay 510 can be used under a corneal LASIK flap and its surface is modified with an excimer laser or the surface of the inlay is modified with an excimer laser prior to implantation or to be placed inside a corneal pocket, and after implantation, the inlay and its surrounding stroma is treated with riboflavin of 0.1-1% solution or more and the eye is irradiated with UV radiation for a short period of time of 1-10 minutes, and the eye is treated with topical anti-inflammatory agents.

In one embodiment, the inlay with a dark central ring is used after a LASIK flap is formed in the host cornea and the flat inlay 500 is positioned over the corneal stroma and its surface can be modified with an excimer laser 522 ablating the entire inlay to correct the refractive error (see FIGS. 39A-39D) of the eye using a wavefront technology and excimer laser 522 while providing a pinhole (ring) inside the cornea, to extend the focal point of the eye for the patient to see far and near or in between and the rest of the clear peripheral inlay 504 is treated with an excimer laser to correct refractive error of the eye such as myopia, hyperopia and astigmatism, then riboflavin solution is applied over the inlay 500 and the corneal flap 520 covers the inlay 500 while the riboflavin penetrates the inlay and the surrounding tissue of the cornea, the inlay and the surrounding tissue is simultaneously crosslinked by irradiating the eye from the outside with UV radiation 524 for 3-9 minutes and a power of laser adjusted at 3-9 miliW/cm2 to crosslink the inlay 500 and the surrounding corneal tissue preventing the rejection of the inlay. In this procedure, the refractive error of the eye is corrected on the peripheral transparent part 504 of the inlay 500 while simultaneously providing a central pinhole to extend the depth of focus for the patient looking at an object in the far or near (see FIGS. 39A-39D) with a biocompatible non-toxic dark or black India ink or acrylic black ink, etc. for correction of the refractive errors of the eye. The inlay procedure is reversible, where the inlay can be removed or replaced in the post-operative period.

In one embodiment, the inlay can be removed prior to a cataract extraction and the refractive error is corrected with an intraocular lens with or without a through central hole.

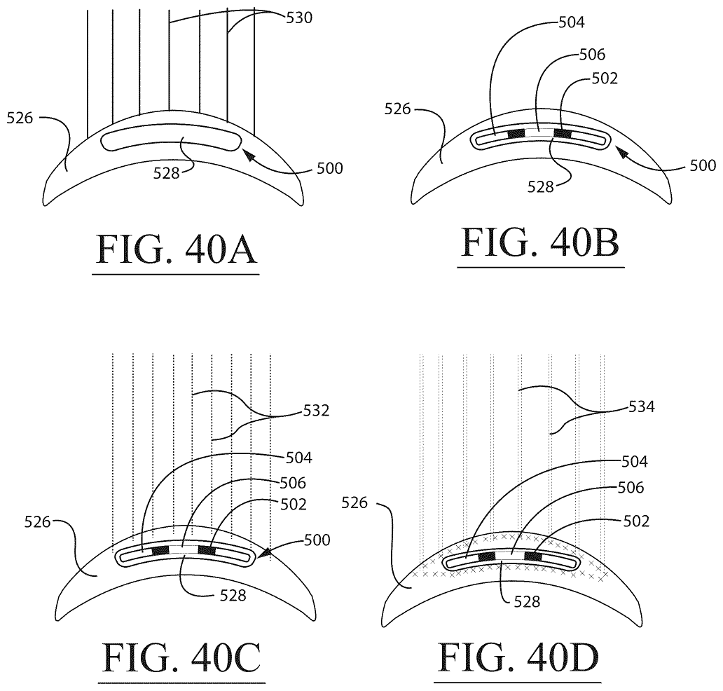

In one embodiment, with a corneal pocket procedure where a pocket 528 is formed in the cornea 526 of the eye using a femtosecond laser 530, the crosslinked corneal inlay 500 with the central hole 506 is implanted in the pocket 528 of the cornea 526, and crosslinked as above, then after a period of time if one desires, one can perform a Photo-Refractive keratectomy (PRK) (see FIGS. 40A-40D) procedure using an excimer laser 532 with wavefront technology on the surface of the cornea 526, limited only to 5-7 mm diameter area of the corneal surface and correct the refractive error of the eye, waiting 1-2 days for the corneal epithelium to heal permitting the eye to see far and near, while preventing the side effect of the standard PRK which are severe pain or loss of corneal sensation for 1-2 months or dry eye since the corneal nerves are partially cut which significantly reduces the pain sensation in this combination procedure with LASIK, SMILE procedure, or PRK. As shown in FIG. 40D, as part of this procedure, the inlay 500 in the pocket 528 is crosslinked by irradiating the eye from the outside with UV radiation 534 for 3-9 minutes and a power of laser adjusted at 3-9 miliW/cm2 to crosslink the inlay 500 and the surrounding corneal tissue preventing the rejection of the inlay.

In one embodiment, the dark ring can be made from the eye bank cornea or genetically modified animal cornea tattooed with a biocompatible non-toxic dark or black India ink or acrylic black ink, or a combination of polyvinylidene fluoride and carbon black nanoparticles, etc., or with PEGylated carbon nonospheres or nanotubes of 5-10 micron long that are mixed with polymeric nanoparticles such as PMMA, hydrogels, silicone, PVDF, polypropylene, polycarbonate, PVC, polysulfone, PEEK, polyethylene, acrylic copolymers, polystyrene, or collagen gel and crosslinked subsequently or its surface can be modified prior to implantation in the corneal stroma and combined with crosslinking the implant and the stromal tissue.

In one embodiment, for 3-D printing an organic corneal inlay and crosslinking it with riboflavin or another photosensitizer and UV radiation while the 3-D structure of the ring with carbon nanotubes or carbon nanoparticles, in acellular collagen or another polymer, absorb 99.99% of the incoming light preventing light from escaping, thus creating a very dark ring inside the inlay with a clear peripheral organic inlay.

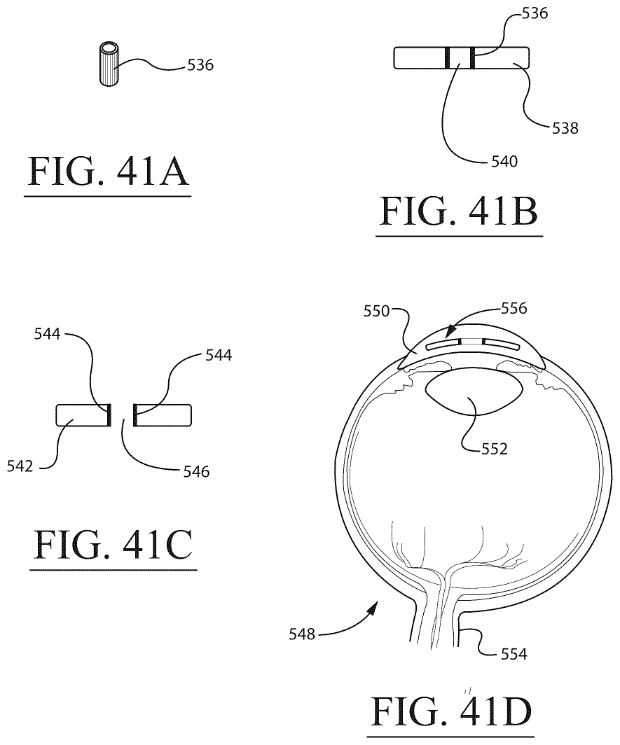

In one embodiment, after 3-D printing or creating an inlay from human or animal cornea, a 1-2 mm wide dark cylinder 536 (see FIG. 41A) with sharp edges and a length of <400 microns, can pushed through the center of the inlay 538 to penetrate the inlay 538 (see FIG. 41B) and stay in place to act as a pinhole 540 with dark wall, and the inlay 538 is crosslinked before or after implantation to act like a pinhole 540 extending the focal point of the eye with or without correcting the refractive surface of the inlay 538.

In one embodiment, a collagenous dark ring can be made by tattooing the cornea with acrylic ink, carbon black or carbon nanoparticles or PEGylated carbon nanoparticles or PEGylated nanotubes of 5-10 micron long that are mixed with polymers such as PMMA, hydrogels, silicone, PVDF, polypropylene, polycarbonate, PVC, polysulfone, PEEK, polyethylene, acrylic copolymers, polystyrene, polyvinyl proline, polyvinyl fluorine or collagen gel and the ring placed over the host corneal stroma, having a LASIK flap before or after correcting the refractive error of the eye on the inlay's surface with an excimer laser and the inlay and the host tissue are crosslinked with the riboflavin and UV radiation to prevent rejection of the inlay.

In one embodiment, an injectable mixture of carbon nanotubes or nanoparticles made from carbon or other material and acrylic polymers etc. can be used for tattooing the corneal inlay at the inlay's central area so that a circular part of its center can be cut away with a trephine or a femtosecond laser, to create the central through and through hole in the inlay while leaving a dark rim around the hole and a clear peripheral donor cornea, that is modified with an excimer laser and crosslinked with riboflavin and UV radiation to be implanted inside a corneal pocket, which is then crosslinked with riboflavin and UV radiation.

In one embodiment, the central 1-2 mm of the transparent corneal inlay is left untouched while the tissue surrounding it is tattooed with an injectable carbon nanoparticles and an acrylic polymer to create a dark circular ring with a total width of 2-3 mm or more functioning as a virtual pinhole 560 (see FIG. 42B), without a through and through actual hole, and the surface of the remaining clear inlay of the 6-9 mm in diameter can be modified with an excimer laser to correct the refractive error of the eye before or after implantation to correct myopia, hyperopia or astigmatism while the central clear acts as a pinhole to extend the focal point of the eye for correction of presbyopia and strengthen the biomechanical property of the cornea.

In one embodiment, a circular ring of a diameter of 1-2 mm or more can be tattooed through the surface of the cornea for a distance of 10-50 microns to create a pinhole in the cornea without implanting an inlay inside the cornea, thereby creating a semi-permanent pinhole for the eye with or without standard corneal crosslinking of human animals to provide them with an extended focal point for far and near.

In one embodiment, after the inlay implantation inside the corneal stroma the inlay and its surrounding tissue is crosslinked with riboflavin and UV radiation to prevent an immune response and rejection of the inlay.

In one embodiment, the central pinhole 546 and its dark surrounding rim 544 of tissue occupies the central 3.5 mm in diameter part of the inlay 542 (see FIG. 41C) which has a peripheral diameter of 3.5-9 mm of clear transparent donor tissue that can be modified to correct refractive error of the eye and crosslinked. In FIG. 41D, the host eye 548 with lens 552, cornea 550, and optic nerve 554 is depicted with a corneal inlay 556 having a darkened wall or implanted darkened cylinder.

In one embodiment, the corneal inlay with a central hole and darkened peripheral hole is made by tattooing from human eye bank, or animal of genetically modified animal or not modified animal cornea or 3-D printer collagen or molded collagen with the diameter of 5 mm is cut with a trephine, or microkeratome, or femtosecond laser, the inlay is tattooed with the inlay is cut with a microkeratome or a femtosecond laser or an excimer laser to a thickness of 10 or more microns and in diameter and the inlay and the surrounding corneal tissue after implantation is crosslinked to prevent rejection.

In one embodiment, the corneal inlay is made from human eye bank eye or genetically modified animal cornea or animal corneal is made non-immunogenic with a combination of riboflavin and riboflavin solution and irradiated with a UV laser to crosslink the RNA, DNA, proteins, and glycoproteins inside the cornea to be modified subsequently with an excimer laser or femtosecond laser for implantation inside the cornea of human or an animal to correct their refractive error and crosslinked the wall of the corneal pocket to prevent their rejection by crosslinking the corneal cavity surrounding the inlay.

In another embodiment, the corneal inlay is made from the animal corneas such as dog, horse, pig or any other animals or an animal whose genes are modified first with recombinant genetic technology to prevent HLA histocompatibility response in human tissue, the inlay is initially de-cellularized with a solution of sodium dodecyl sulfate and or benzalkonium chloride, and/or treated with riboflavin and methylene blue photosensitizers and UV radiation to crosslink all cellular protein, glycoprotein and RNA and DNA of the cells and make the tissue non-antigenic while the inlay remains transparent, but permits transport of water and other molecules through it, it is then processed for providing a central through hole and with or without tattooing the edges of the holes or implanting a thin polymeric darkened ring or a combination of polyvinylidene fluoride and carbon nanoparticles inside the inlay having the same thickness as the inlay and can be ablated with an excimer laser to modify the refractive error of the eye and after implantation and the central part of the inlay does not lift up selectively the central area of the cornea and finally the inlay and tissue surrounding the inlay are also crosslinked to kill the corneal cells and pathogens, creating an immune privileged space for the inlay implantation while correcting the refractive error for far and near and enhancing the mechanical stability of the host cornea.

In one embodiment, the animal cornea or human corneas is decellularized chemically e.g. sodium dodecyl and or undergoes a corneal crosslinking with 0.1% to 5% riboflavin and/or methylene blue at concentration of 4 mg/L or more and UV radiation at 320-380 nm wavelength and the power of 3-10 mw/cm2 to crosslink the acellular organic inlay with intercellular and cellular protein, glycoproteins and RNA or DNA, damaging all cells, bacteria, viruses, or parasites in the cornea, sterilizing the inlay maintaining the transparency of the inlay and simultaneously preventing an immune response to the inlay.

In one embodiment, the inlay is crosslinked, then is ablated with an excimer laser equipped with a wavefront technology to correct precisely the refractive power of the eye after the inlay is implanted inside the host cornea and crosslinked.

In one embodiment, the refractive power of the inlay is corrected prior to the corneal crosslinking, using an excimer laser and wavefront technology and Shack-Hartmann sensor to reshape the inlay and to correct refractive error of the patient after implantation, the inlay is crosslinked with riboflavin alone or in combination with methylene blue and irradiated with UV radiation killing the cells and potential bacteria, viruses or parasites in the corneal inlay then is implanted inside a corneal pocket created by a femtosecond laser in the host cornea, then Riboflavin is injected in the corneal pocket over the inlay and the surrounding corneal inlay is crosslinked with UV radiation from the outside the cornea to prevent an immune response from the host cornea.

In one embodiment, the synthetic organic corneal inlay is made of collagen or a combination with other organic materials with or without a central hole 3-D printed or molded as described above to which a darkened polymeric ring may be simultaneously 3-D printed inside the collagen molecules which will have a transparent peripheral part while an acrylic India ink, etc. is injected with collagen in process of 3-D printing using a computer to form a 2-4 mm flat darkened ring in the center of the inlay with a transparent central part including polymeric organic or synthetic non-magnetic materials with a thickness of 10-50 micron or more, while the central opening or virtual aperture in the ring is between 1-2 mm and having a rim of 1-2 mm surrounded by the optically transparent collagen with the diameter of 5-9 mm to which riboflavin at a concentration of 0.1-1% may be added after the inlay is formed, if the inlay needs to be crosslinked with UV radiation prior or after it implantation. The inlay can be used with a LASIK flap and its surface may be modified with an excimer laser or the surface of the inlay is modified with an excimer laser prior to implantation to be placed inside a corneal pocket after implantation the inlay is treated with riboflavin or a 0.1-1% solution or more and the eye is irradiated with UV radiation.

In one embodiment, the procedure is as LASIK procedure, thus creating a corneal flap with a microkeratome or an excimer laser.

In another embodiment, the corneal inlay with the central hole and dark wall is implanted in a corneal pocket of human or animals such as horse dog, etc. which is created by a femtosecond laser application inside the corneal stroma at desired level inside the stroma.

In one embodiment, prior to the implantation of the corneal inlay a central hole is created in the inlay then the circular polymeric dark ring is place inside the inlay's producing through and through central hole so that the ring and the inlay have the same thickness and after their implantation the corneal flap is not elevated forward and increases the mechanical stability of the cornea in high myopia and or in keratoconus.

In one embodiment, the wall of the hole in the inlay is tattooed for a diameter of 0.5-2 mm or more as needed, the dark tattoo can be applied uniformly to either surface of the inlay with dark non-toxic particles such as carbon, the tattooing can be done before cutting out the central part of the inlay with a trephine or a femtosecond laser, leaving a the dark rim of tattooed tissue.

In the femtosecond SMILE pocket procedure, the refractive power of the eye is corrected on the inlay with the central hole prior to its implantation and the inlay and the corneal wall surrounding it is crosslinked by injection of riboflavin solution inside the corneal pocket and the eye is irradiated with UV laser light from the outside, to crosslink the inlay and the corneal tissue preventing rejection of the inlay while correcting for presbyopia and other refractive errors of the eye.

In one embodiment, in the LASIK flap procedure, the inlay with its central opening is placed over the corneal stromal after creating a corneal flap and the inlay's refractive power is corrected with an excimer laser by ablating the inlay's surface, and crosslinking it, strengthening the biomechanical stability of the cornea with the inlay in high myopia or eyes with keratoconus.

In one embodiment, in a corneal flap procedure, after correction of the refractive power of the eye on the inlay, it is crosslinked by placing a few drops of a solution of a photosensitizer, such as riboflavin, with or without methylene blue over the cornea and the corneal flap is repositioned over it, the cornea is crosslinked with UV radiation done from outside the eye, crosslinking the inlay and the surrounding stromal tissue of the flap and the corneal stroma to prevent rejection of the inlay.

In one embodiment, after making a corneal pocket with a femtosecond laser, the excimer laser ablated corneal inlay with its central ring or darkened tissue around the central opening, is implanted inside the pocket so that the central hole coincides with optical axis of the eye then a few drops of riboflavin at 0.1-1% solution alone or in combination with methylene blue at <2 mg/L concentration are injected over the inlay to penetrate it and the surrounding corneal tissue, then the eye is irradiated with UV radiation to kill all cells in the inlay or surrounding cornea along with potential bacteria, viruses or parasites while crosslinking the host corneal tissue around the corneal pocket.

In both LASIK or SMILE corneal pocket procedure, an antibiotic or an anti-inflammatory agent is applied to the cornea or injected in the corneal pocket to protect the cornea from infection, or deliver the medications by using slow release polylactic or glycolic or combination of them or other slow release compound and release them for 4-6 weeks.

In one embodiment, an inlay with a desired thickness of 20 micron to 500 micron or more and a diameter of 6-9 mm is obtained from the human eye bank or genetically modified animal or not modified, such as pig or molded or 3D printed from collagen, etc. and crosslinked to make them non-immunogenic for transplantation. The inlay may also be produced as a synthetic collagen inlay, etc.

In one embodiment, the corneal inlay made of human or genetically modified animal cornea, crosslinked and its surface is modified as described, one can implant it in almost any depth from the surface of the host cornea and does not need to be implanted at a depth beyond 120-200 microns from the surface, which cannot transmit the change of the inlay surface to the corneal surface precisely which is important in refractive surgery this is followed with crosslinking the tissue around the inlay as described above.

In one embodiment, the corneal inlay made of a human or genetically modified animal cornea, or synthetically produced from collagen and other polymers may be crosslinked and its surface may be modified as described above. The inlay may be implanted after the correction of its refractive power as needed with an excimer laser, then implanted in the corneal pocket in almost any depth from the surface of the host cornea and does not need to be implanted at a depth beyond 120-200 microns from the surface, a deeper pocket that has a front surface tissue >150-200 micron from the surface may not sufficiently deform to transmit the change of the inlay's surface to the corneal surface precisely. This may be followed with crosslinking the tissue around the inlay as described above prior to the implantation.

In one embodiment, all refractive surgeries in human or animals can be done on the corneal inlay with central hole can be repeated again by replacing the old inlay with a new corneal inlay with a central hole to treat refractive errors of the eye and presbyopia simultaneously or perform a bilateral procedure permitting the patient to see stereoscopically the objects located at different focal points in front the eye and regardless of its original eye's dioptric power or age of the patient for correction of myopia, hyperopia, stigmatism, and presbyopia since no tissue is removed from the cornea, without the risk of rejection of the crosslinked inlay surrounded by crosslinked host corneal tissue and without causing corneal haze.

In one embodiment, the inlay is used to correct hyperopia by increasing the convexity of the central part of the cornea. In this embodiment, the implant is a small decellularized corneal inlay with the diameter of 2-4 mm and a thickness of 10 to 40 microns with a central hole of 1-2 mm and a tattooed 1-2 mm peripheral rim that can be implanted in a cavity of 5 mm in diameter made in the cornea with a femtosecond laser to achieve a precise dioptric power in the center of the retina which create addition convex curvature in the center with a pinhole effect with or without crosslinking the inlay and the corneal tissue achieving simultaneously a bifocal near and far vision for the patient or animal. The inlay and the surrounding tissue is crosslinked with a solution of riboflavin with or without methylene blue and UV laser radiation to prevent rejection of the inlay and simultaneous crosslinking of the adjacent corneal tissue.

In one embodiment, the pinhole is oval with a vertical diameter of 1-2 mm and horizontal diameter of 1.5 mm-3 mm with the dark wall made of polyvinylidene fluoride carbon black or polyester derived from chemical reaction of polyester (polyethylene terephthalate).

In one embodiment, the synthetic corneal inlay is made from collagen and 3-D printed to any shape desired for the corneal inlay to correct hyperopia, and its refractive error is corrected as needed with an excimer laser and femtosecond laser to be implanted after making a SMILE pocket in the corneal stroma where a part of tissue is removed to create space for the inlay implant having a central hole with a surrounding dark pinhole or without a hole.

In one embodiment, the above-described organic inlays with a central hole can be used for myopia, hyperopia, and/or astigmatism, etc. for any age group of patients even after cataract surgery or previous refractive surgery to create a multifocal view from far to near for the patient's eyes.

In one embodiment, in the postoperative period, topical anti-inflammatory agents along with antibiotics or nerve growth factors and Rock inhibitors alone or with anti-VEGFs are applied as topical drops or with slow release compounds to enhance corneal nerve regeneration.

In one embodiment, the synthetic cornea can be used for implantation in the human eye that requires refractive surgery.

In one embodiment, the synthetic inlay is made to be placed inside the human cornea to correct the refractive error of the eye where corneal inlay is cut as a circle or oval or squared or another shape for implantation the inside the host cornea, inside a corneal pocket or a under a LASIK flap. The synthetic or organic lens is either molded or 3-D printed to have a refractive function, e.g., for correction of myopia with spherical aberration. In this embodiment, the circular inlay may be molded or 3-D printed having a diameter of 5-7 mm and various thicknesses of 10-200 microns or more and its surface is either shaved by milling or using a keratome or using an excimer laser to ablate its surface or a femtosecond laser to cut a desired part of the inlay by removing a central part of the inlay to function as a myopic lens with −0.25 to −10.00 or −15 or −20.00 diopter power or more, or the lens's refractive power if modified using a femtosecond laser to cut the superior part of the lens and removing a desired amount of the lens to become a minus lens of desired refractive power to be implanted inside a LASIK flap or inside a corneal pocket, as in a SMILE procedure, with or without removing any host tissue, thereby the inlay acts as a minus lens for implantation inside the patient's cornea to correct the myopic refraction of the patient, or one can create a convex lens with the laser ablating or cutting away from the inlay for implantation inside an hyperopic host cornea where the lens is prepared with a flat surface lying over the corneal stroma and the convex side (surface) toward the corneal pocket or LASIK flap, or for correction of astigmatism, an astigmatic lens may be formed using a femtosecond laser or an excimer laser to cut or ablate the synthetic inlay with a desired axis and power where the lens is implanted so that the flat surface lays on the patient corneal stroma and the refractive surface toward the corneal flap or LASIK flap created with a femtosecond laser where the upper corneal layer (flap) has the corneal epithelium.

In one embodiment, if one has access to a femtosecond laser or preferably an excimer laser, the implant can be placed over the surface of the corneal stroma after producing a corneal LASIK flap and the surface of the inlay is modified with a femtosecond laser or an excimer laser to obtain the desired refractive power under the control of a Shack-Hartmann sensor to precisely correct the needed refractive power of the inlay and the eye as a whole.

In one embodiment, the synthetic inlay is 3-D printed so that the central part of the inlay has a circular ring of 1-2 mm made of a mixture of collagen and polyvinylidene fluoride and black carbon particles to produce a virtual pinhole with a central clear area of 1-2 mm and a black wall located inside the transparent synthetic, organic, or eye bank inlay acting as a pinhole.

In one embodiment, the 1-3 mm central area of the inlay is printed with darkened polyvinylidene fluoride carbon black or another polymer, such as polyethylene terephthalate black carbon, where a central through hole can be cut away using a trephine of desired sized diameter leaving a central through pinhole or an oval pinhole with a horizontal diameter of 2-2.5 mm and a vertical diameter of 1-2 mm as desired to eliminate problems of decentration of the pinhole inlay with its dark wall.

In another embodiment, a pinhole is cut and removed inside the center of the inlay with a femtosecond laser to create a desired shaped pinhole, such as round or oval, etc.

In one embodiment, a synthetic inlay, a 3-D printed organic inlay, or an organic collagen inlay with a central pinhole of 1-2.5 mm and a darkened wall is used in deep lamellar keratoplasty deep inside the host cornea behind the Descemet's membrane, as known as Descemet's Membrane Endothelial Keratoplasty (DMEK), where the synthetic or organic cornea has a uniform thickness of 10-70 microns or thicker and a diameter of 3-7 mm.

In one embodiment of synthetic, organic collagen, or 3-D printed corneal implantation, a corneal crosslinking procedure is performed using riboflavin and UV radiation during or in the early postoperative period, and to remove any existing sutures within a reasonable time of 1-2 months or more to prevent postoperative immune response with migration of macrophages, t-cell leucocytes, and lymphocytes and humoral response with TNF production, etc. Any inflammatory response in the postoperative period should be addressed with topical administration of Rock, GSK, integrin, and/or Wnt inhibitors with or without anti-VEGF drops and an antibiotic to prevent an infection. In addition, corneal crosslinking adds to the mechanical stability of the inlay and the cornea, which otherwise with time loosens the stability of the connections of the implant with the edges at the host tissue interface. For this reason alone, a synthetic or organic implant is more stable inside the existing tissue after corneal crosslinking and becomes less erodible. In addition, corneal crosslinking kills potential pathogens, such as bacteria, viruses, etc.

In one embodiment, the genetically modified cornea can be used for full thickness corneal transplantation while crosslinking one-half or more of the thickness of the cornea with riboflavin and UV radiation.

In one embodiment, the ablatable corneal inlay for simultaneously correcting refractive errors and presbyopia is provided with a virtual hole or actual hole surrounded by a clear transparent fluid which is a permeable organic or non-organic composition, etc.

In one embodiment, using a method of corneal inlay implantation where the inlay can be made either from collagen, molded, or 3-D printed to the desired shape or it is made from corneal stromal tissue harvested from human eye bank eyes, from deceased patients, or from animals, such as pigs, etc., with genetic modification or the animal eyes are obtained from the slaughter house, then prepared so that their nucleus and proteins, etc. lose their immunogenicity using high concentration of methylene blue >4 microgram/ml combined with peptide nucleic acid and/or PARP inhibitors to block the cells RNA and mRNA of the corneal cells or pathogens, in a solution with high osmolality of more than 300 mOS, that both damages the nucleic acid and withdraws fluid from the cornea or combined with riboflavin, irradiated with UV radiation or a wavelength that is absorbed by methylene blue (MB) at 660 nm to crosslink the antigenic proteins, glycoproteins of the corneal inlay, reduce or eliminate their antigenicity prior to implantation or during the implantation in the cornea after radiation with UV or another wavelength, etc.

In one embodiment the human or animal corneas are separated from the sclera, conjunctiva, or retina, etc., stored in a solution, such as preservatives, to kill and eliminate the cellular elements or pathogens as described in U.S. Pat. No. 10,881,503 to Peyman using Benzalkonium chloride (BAC) solution or with 0.1% sodium dodecyl sulfate (SDS) or combination, etc. and washed in a physiological solution with slightly higher osmolarity to reduce toxicity of SDS, followed by washing, etc. U.S. Pat. No. 10,881,503 to Peyman is incorporated by reference herein in its entirety.

In one embodiment, the de-cellularized corneas are cut either with a microkeratome or a femtosecond laser, etc., and kept moist with Benzalkonium chloride in hyaluronic acid or low molecular weight heparin or albumin so that the tissue does not swell.

In one embodiment, uniform corneal stromal inlays are prepared with the thickness of 40 microns to 400 microns with a diameter of 3-9 mm, preferably a diameter of 5-8 mm or more, with a 30-150 micron thickness or more, and the inlays can be exposed to UV or cobalt radiation, etc. if needed to eliminate bacteria, fungi, or viruses or parasites, or exposed to high concentration of methylene blue >3 microgram/ml with or without β-propiolactone (BPL) or a mixture of methylene blue (MB) at a concentration of >4 microgram/ml for a period of time 1-5 hours or more to damage RNA and DNA of the inlay or any organism, or crosslinked with riboflavin and UV radiation wavelength of about 370, or wavelength of about 650 nm for MB crosslinking of the inlay, etc., and to damage potential organisms, viruses, bacteria, fungi or parasites in the inlay, or crosslinking can be performed after implantation in the corneal stroma.

In one embodiment, one predetermines the refractive error of the patients with various means such as phoropter, wavefront guided excimer laser technology, or an automated objective phoropter (see e.g., U.S. Pat. No. 8,409,278, which is incorporated by reference herein in its entirety) that provides a correction to the refractive error of the eye in less than 5 seconds, the prescription is printed out by the unit prior to corrections of refractive errors with the refractive error being corrected with wavefront guided excimer laser prior to its implantation.

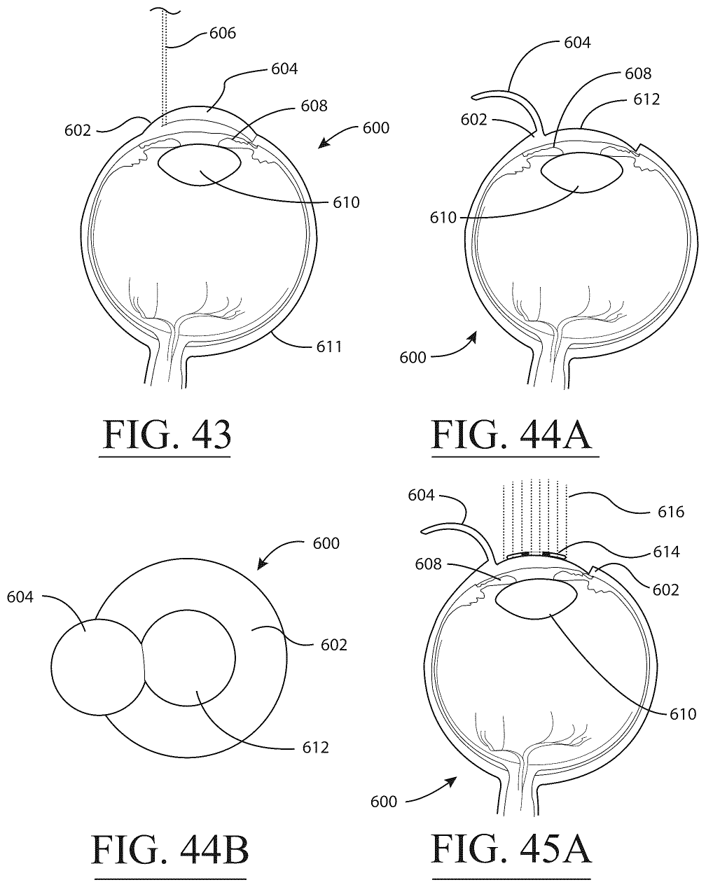

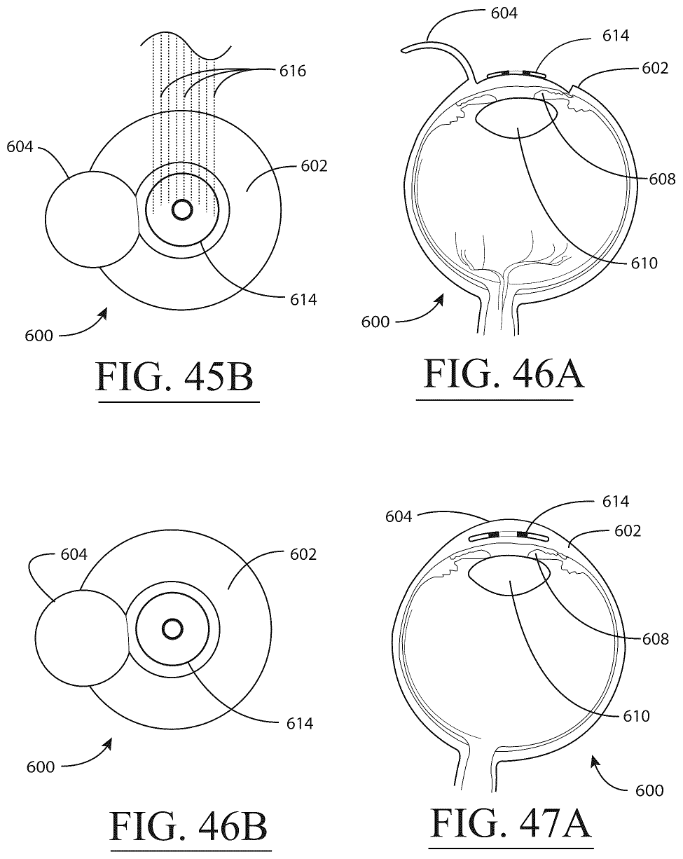

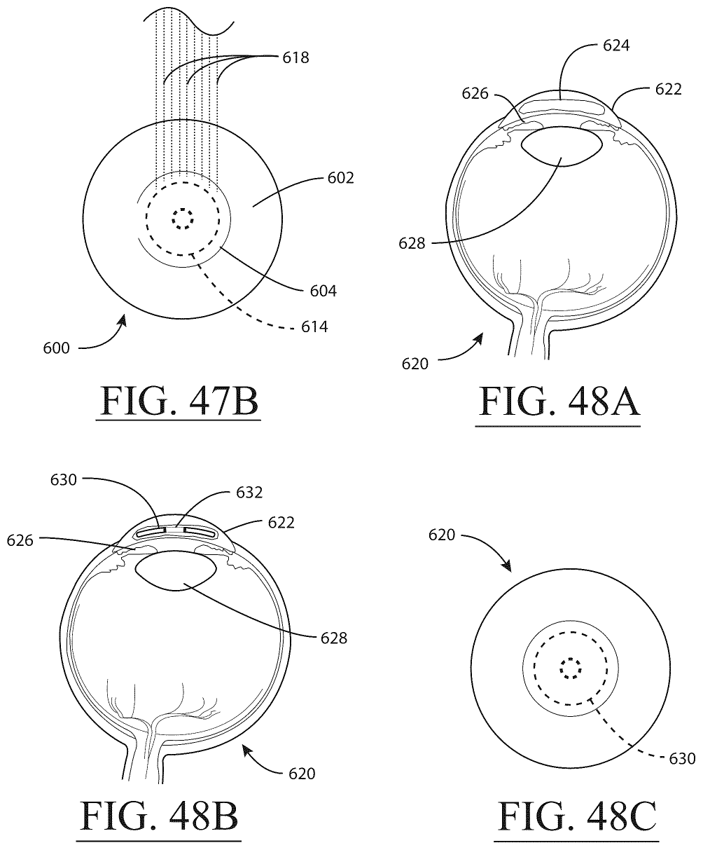

In one embodiment, a standard LASIK flap is produced either by a microkeratome or a femtosecond laser, etc. (see FIGS. 43, 44A, and 44B). For example, referring to FIG. 43, it can be seen that a corneal flap 604 is being formed in a host eye 600 with a cornea 602, an iris 608, a lens 610, and a sclera 611 using a femtosecond laser 606. In FIGS. 44A and 44B, the corneal flap 604 is pivoted, thereby exposing the stromal tissue 612 of the cornea 602 underneath the flap 604. Then, turning to FIGS. 45A and 45B, a corneal inlay 614 with a central pinhole is placed on the stromal tissue 612, and the corneal inlay 614 and stromal tissue 612 is ablated using an excimer laser 616. Next, as illustrated in FIGS. 47A and 47B, the corneal flap 604 is repositioned, and ultraviolet (UV) radiation 618 is applied to crosslink the corneal inlay 614 and the surrounding corneal tissue preventing the rejection of the inlay 614.

A de-cellularized and sterile corneal inlay is selected with the size and/or thickness depending on the need of the eye to create emmetropia correcting both spherical and cylindrical errors, etc. as needed for the patient.

In one embodiment, the corneal inlay is positioned on the exposed corneal stroma, under a prepared LASIK flap. Initially, the inlay is treated with an excimer laser and/or femtosecond laser to provide a uniform size and thickness of the corneal inlay, e.g., 8-9 mm or more in diameter and 30 microns or more in thickness, e.g., for correcting a −5.00 diopter error, the inlay is ablated in its center on an area of 4-6 mm with an excimer laser, calculated based on a mathematical formula and laser software where removal of each 10 microns of the circular inlay's surface creates one dioptric power change. In the present procedure, the surgeon divides the 5.00 dioptric ablation between the corneal inlay and the patient's stroma. The correction of the inlay for −3.00 dioptric power or 30 micron ablation is done on the center of the donor inlay and the remaining −2.00 D power is done on the patient's stroma (total amount of tissue removed is 50 microns, which equals −5.00 D power-refer to FIGS. 43-47B). Similarly, in another example for-10 diopter correction, 100 microns of stromal tissue removal is required. However, one ablates an 80 micron thickness ablation on the central 6 mm part of the inlay having a thickness of 80 microns and a diameter of 8 mm and can also ablate the patient's stroma for additional 20 microns beyond the inlay. Therefore, flattening the surface of the central cornea for total of −10.00 D by removing 80 micron thickness from the corneal inlay and only 20 microns from the patient's corneal stroma instead of the 100 microns without the inlay.

In one embodiment, the ratio of ablation of the inlay versus the corneal stroma can be any ratio, e.g., creating 50%/50% more or less ablation producing potentially >15.00 dioptric power correction. However, it is more desirable to ablate more on the donor corneal inlay than the corneal stroma etc. depending on the dioptric power needed to fully correct refractive error of the eye.

This method of dividing the amount of corneal ablation between the inlay and the host corneal stroma creates a hole in the inlay by the initial ablation, through which the patient's cornea is exposed, which is then ablated for 20 microns or about −2.00 dioptric or more for additional correction. Then, when the transparent corneal flap is repositioned over the inlay, a part of the flap comes directly in contact with the transparent patient's stroma underneath it or through the doughnut hole of the inlay. The patient's eye corrected with this technology (e.g., which may be called a Tissue-Augmented (TA) LASIK procedure) can immediately see the outside world clearly, since there is no tissue in the center of the doughnut-shaped inlay.

In one embodiment, this procedure can be repeated, or the ablation can be modified or the new inlay can be placed over the old one or the old inlay can be removed and a new one inserted in its place with ease in the cornea of a patient's eye that is growing, etc.

In one embodiment, the advantage of Tissue-Augmented LASIK Surgery (TALS or TA LASIK) over the ablation of the inlay only, is that the central opening (doughnut) created after the excimer laser surgery in the donor corneal inlay always remains clear, even if the remaining peripheral inlay tissue is initially less transparent (because of the absorption of water or other medication during the inlay preparation, rendering the inlay tissue slightly less transparent). However, in 1-2 weeks, the inlay becomes as fully transparent as the rest of the cornea. Ultimately, the central ablated hole in the inlay (doughnut) provides the patient with a clear window immediately after the TA LASIK procedure, through which the outside world can be seen or the patient can read clearly immediately after surgery as is the case after LASIK surgery which is not tissue augmented and is limited to 7.00 D power.

In one or more embodiments, the Tissue-Augmented LASIK Surgery (TALS or TA LASIK) procedure is done by augmenting the thickness of the cornea uniformly prior to laser ablation using a part of an eye bank cornea (e.g., about a 100 micron thickness or more and about a 6 mm in circular diameter to make a corneal inlay from the eye bank cornea).

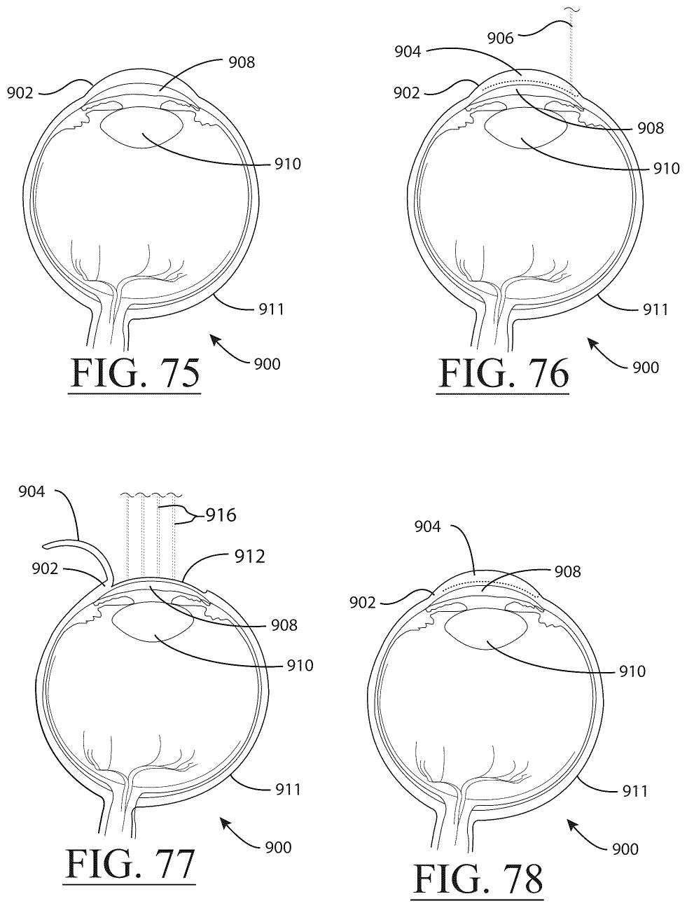

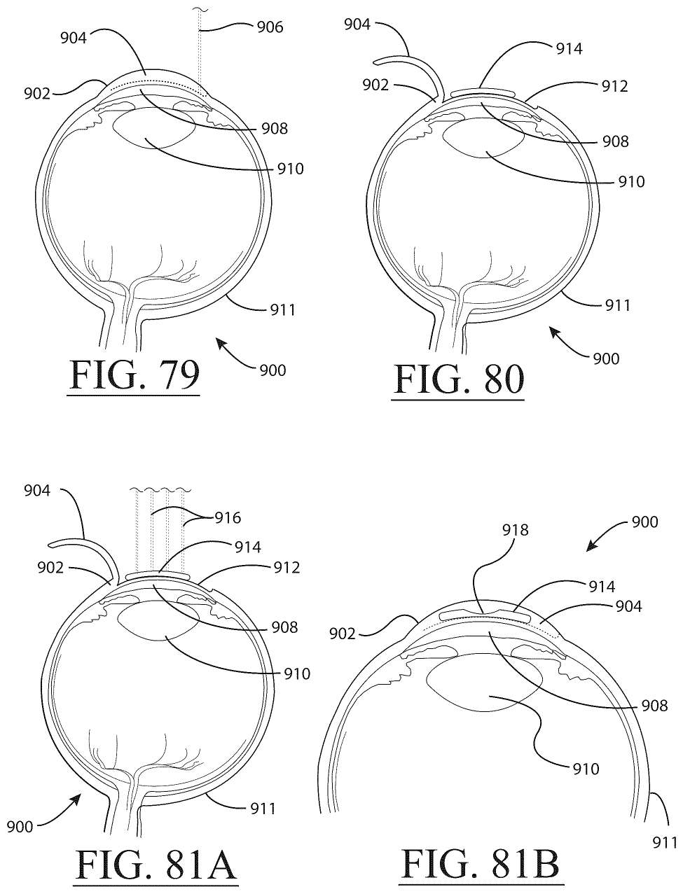

With reference to FIGS. 75-85, illustrative embodiments of a break-through-tissue augmented LASIK procedure (i.e., B-TTAL procedure) will be described. Initially, a LASIK procedure without an inlay is shown in FIGS. 75-78. In FIG. 75, a side view of an eye 900 is shown before the LASIK flap has been cut in the cornea 902 of the eye 900. As shown in FIG. 75, the eye 900 includes an anterior chamber 908, a lens 910, and a sclera 911. In FIG. 76, the LASIK flap 904 is shown being cut in the cornea 902 of the eye 900 of FIG. 75 using a femtosecond laser 906. In FIG. 77, an excimer laser ablation 916 of the corneal stromal tissue 912 under the flap 904 with automated wavefront technology is shown. Alternatively, femtosecond laser cutting of the cornea could be performed using automated wavefront technology. Then, in FIG. 78, the LASIK flap 904 is rotated back into place on the cornea 902 of the eye 900. Next, turning to FIGS. 79-81B, a procedure involving an ablation of the corneal inlay only is depicted. In FIG. 79, the LASIK flap 904 is shown being cut in the cornea 902 of the eye 900 using a femtosecond laser 906. Then, in FIG. 80, after the LASIK flap 904 has been rotated to its open position exposing the corneal stroma 912, a corneal inlay 914 is placed over the corneal stroma 912. After which, in FIG. 81A, an excimer laser 916 is shown being used to only ablate the corneal inlay 914. FIG. 81B is an enlarged detail view of the corneal inlay 914 and a portion of the eye 900 of FIG. 81A, which clearly illustrates that the ablated concave portion 918 of the inlay 914 does not extend into the corneal stroma 912 beneath the inlay 914.

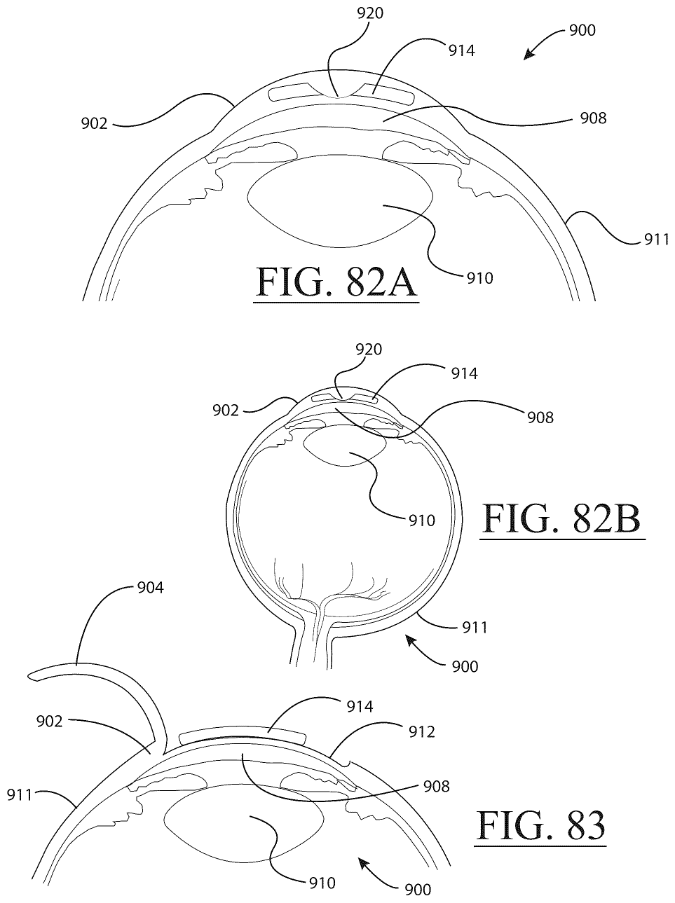

In the illustrative embodiment of FIGS. 82A and 82B, the entire thickness (100%) of the corneal inlay 914 is ablated together with 10 to 20 microns or more of the cornea 902 behind the inlay. FIG. 82A is an enlarged detail view of the corneal inlay 914 and a portion of the eye 900, which illustrates that the ablated concave portion 920 extends entirely through the corneal inlay 914 and into a small thickness of the remaining posterior portion of the cornea 902. FIG. 82B is a side cross-sectional view of the eye of FIG. 82A having the inlay 914, cornea 902, and corneal flap disposed over the inlay 914.

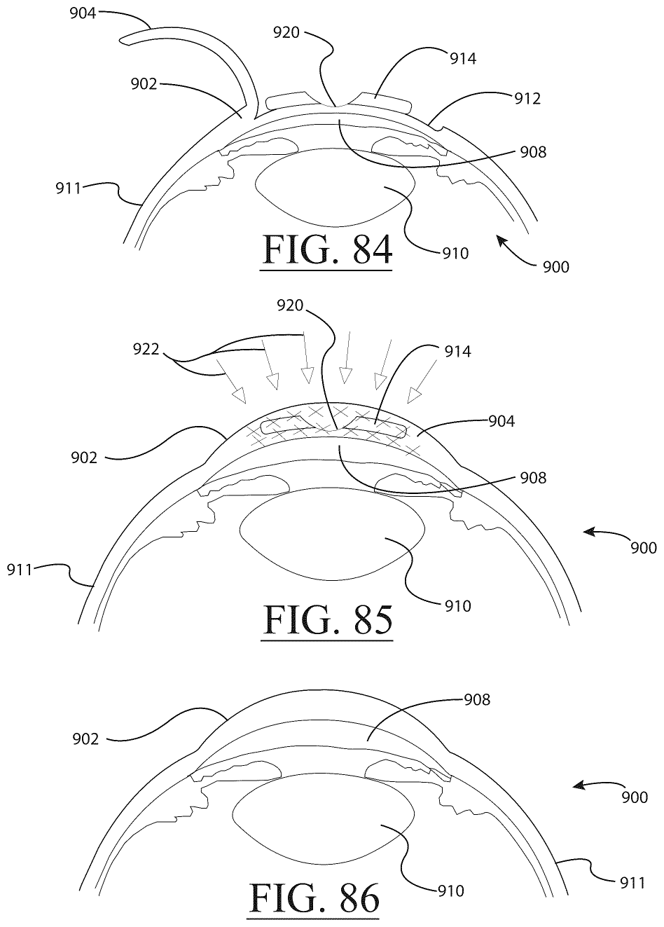

In the illustrative embodiment of FIGS. 83-85, the entire thickness (100%) of the corneal inlay 914 is ablated together with a portion of the cornea 902 behind the inlay 914. In FIG. 83, after the LASIK flap 904 has been rotated to its open position exposing the interior cornea 912, a corneal inlay 914 is placed over the cornea 902. Then, in FIG. 84, an excimer laser is used to cut through the entire thickness of the inlay 914 and a stromal layer 912 of the cornea 902 behind the inlay 914. After which, in FIG. 85, the corneal flap 904 has been replaced, and the concave ablation 920 through the entire thickness of the corneal inlay 914 and a small thickness of the stromal layer 912 of the cornea 902 is visible.

In the illustrative embodiments of FIGS. 79-85 described above, the corneal inlay can be modified either during surgery or prior to surgery, and then implanted in the cornea. In these embodiments, as shown in FIG. 85, the cornea 902 and corneal inlay 914 may be crosslinked using ultraviolet (UV) radiation 922 at the end of the surgery so as to (i) prevent an immune response to the corneal inlay 914, and (ii) increase the strength of the cornea 902 and provide mechanical resistance to deformation. In one or more other embodiments described hereinafter, the entire procedure after implantation of an inlay 914 can be done through a small incision with a femtosecond laser under the control of automated wavefront technology.

In one embodiment, a tissue augmented technique is utilized in order to correct myopia up to −15.00 D. or more or hyperopia of +6.00 D or more without affecting the mechanical stability of the cornea.

In one embodiment in the correction of myopia, a circular or rectangular corneal inlay is prepared with the diameter of 5-6 mm or more and a thickness of 50 microns to 150 microns or more, and the inlay is placed inside the corneal pocket which has a diameter larger than the inlay, such as a diameter of 6.5 mm or larger, and is cut using a femtosecond laser. The circular or rectangular inlay is implanted in the corneal pocket for myopic correction.

In one embodiment in the correction of myopia, a circular or rectangular corneal inlay is prepared with the diameter of 5-6 mm or more and a thickness of 50 microns to 150 microns or more. In this embodiment, a central through hole is created with a diameter of 1-3 mm and the edges of the hole are tattooed with polyvinylidene fluoride and carbon black to darken the wall of the pinhole through which the patient can see far and near. The inlay is placed inside the corneal pocket which has a diameter larger than the inlay, such as a diameter of 6.5 mm or larger. The corneal pocket is cut using a femtosecond laser so as to form the cavity in which the circular or rectangular inlay is implanted.

In one embodiment, the inlay is made either from a human eye bank cornea, or animal cornea, or 3-D printed from synthetic collagen, etc. The corneal inlay may be made acellular by chemical medication or using riboflavin and/or a methylene blue photosensitizer, etc. and appropriate radiation to crosslink the inlay and prevent an immune response by the host corneal tissue. In one embodiment, the riboflavin is injected in the corneal pocket over the inlay, then irradiated with a laser to crosslink the inlay cells and the surrounding host tissue cells, thereby eliminating tissue rejection and killing bacteria, viruses, or parasites, etc.

In one embodiment, either a laser generates two small separate bubbles one parallel to the anterior surface of the inlay and the other curved back side, and the posterior edges of the inlay are ablated including at least two millimeter areas of the posterior host cornea for a thickness of 10-20 microns inside the patient's cornea (see e.g., FIGS. 86-90), and the entire cut area is removed with forceps. This creates a myopic correction for the eye depending on the refractive power needed.

In one embodiment, a single femtosecond beam cuts the posterior surface of a part of the inlay and then moves anteriorly to cut the anterior surface of the inlay under the control of software as shown in, for example, FIGS. 86-90.

In one embodiment for correction of presbyopia and hyperopia, the position of the cut of the inlay is reversed creating a slightly convex lens with the central thickness of about 60 microns or more for correction of hyperopia creating a more convex lens, with the flat surface posteriorly towards the anterior chamber creating a convex lens with a central pinhole of 1-3 mm using a femtosecond laser or a trephine with the darkened edges as described with dark tattooed microparticles for far and near vision, and a hump surface anteriorly for the inlay (see the side view of FIG. 92). Also, the peripheral portion of the inlay may be cut to create a slope toward the periphery of the inlay (refer to the side view of FIG. 92 and the top view in FIG. 91). Alternatively, if the corneal thickness permits a piece of the corneal tissue shaped like that shown in FIG. 92, it can be removed from the corneal stroma with the same effect and the central hole or cylinder is removed from the central corneal stroma and its wall is tattooed as described above. However, the latter requires removal of a significant part of the corneal stroma which might affect the corneal biomechanics even when used in these described technologies, the cornea with its inlay or without inlay is crosslinked.

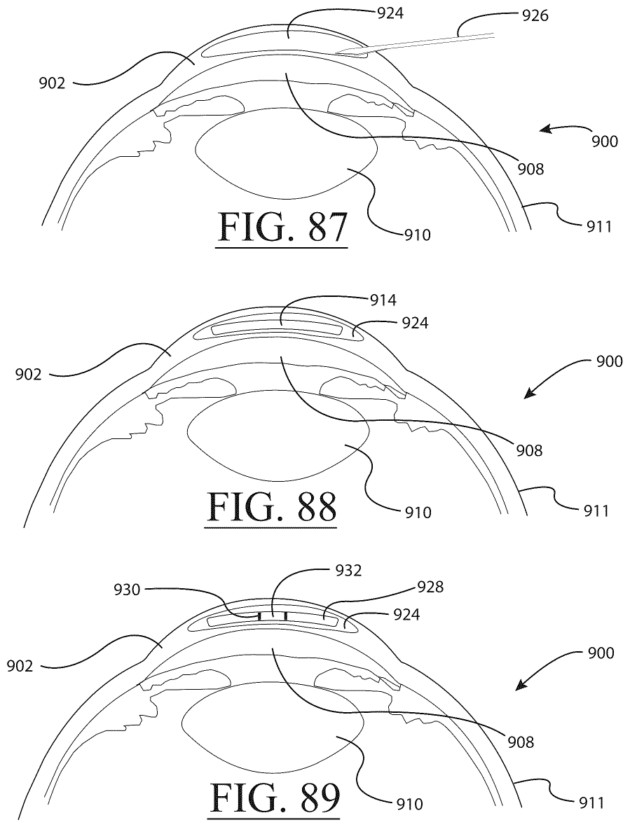

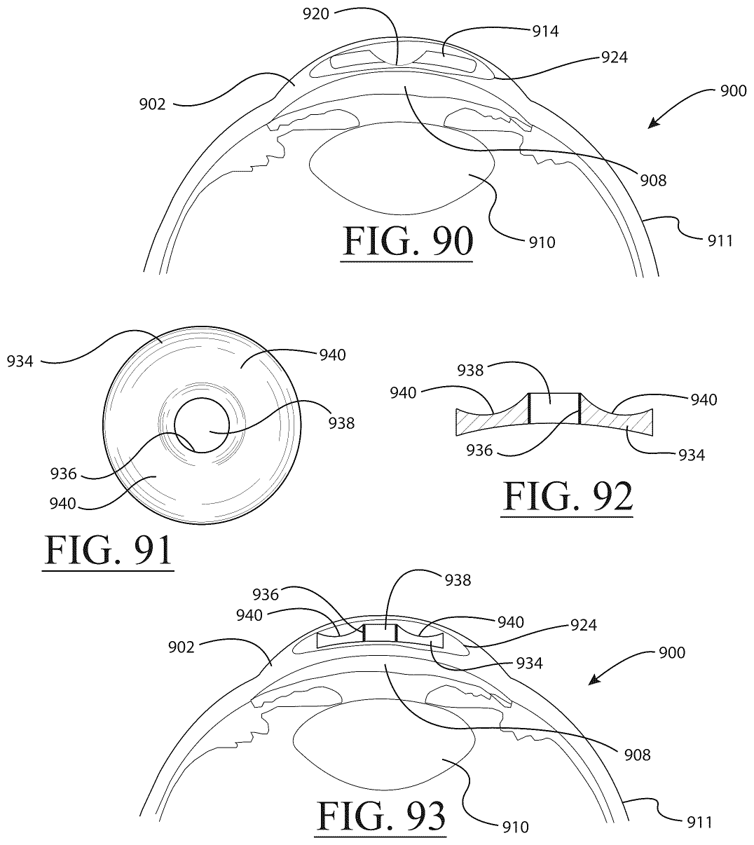

Now, with reference to FIGS. 86-93, illustrative embodiments of in situ correction of myopia with a tissue-augmented SMILE procedure will be described. In FIG. 86, a side view of an eye 900 is shown before the corneal pocket has been cut in the cornea 902 of the eye 900. In FIG. 87, the corneal pocket 924 is shown being cut in the cornea 902 of the eye 900 of FIG. 86 using a spatula knife 926. Alternatively, the corneal pocket 924 may be cut into the cornea 902 of the eye 900 using a femtosecond laser. In particular, in this embodiment, the femtosecond laser is initially used to cut a peripheral boundary around a three-dimensional interior section of corneal tissue that is to be removed. Then, a small incision is made from outside to gain access to this three-dimensional interior section of corneal tissue. After which, forceps are inserted through the small incision to grasp and remove the three-dimensional interior section of cut corneal tissue, while the anterior cornea remains intact over the corneal pocket. Then, in FIG. 88, after the corneal pocket 924 has been formed, a corneal inlay 914 is placed inside the corneal pocket 924. As shown in FIG. 89, in another embodiment, the corneal inlay 928 may comprise a central pinhole 932 bounded by a darkened peripheral wall 930 circumscribing the pinhole 932. Next, referring to FIG. 90, a femtosecond laser may be used to cut through the entire thickness of the inlay 914 and into the posterior stroma behind the inlay 914. For example, in the embodiment of FIG. 90, the inlay may form approximately 80% of the tissue cut by the femtosecond laser and removed with forceps, and the anterior surface of the corneal stroma may approximately form the other 20% or more of the tissue cut and/or ablated by the femtosecond laser. In these embodiments, the femtosecond laser cuts the outline in the tissue, and cuts the corneal inlay (e.g., 80% of the total thickness) and the posterior layer (e.g., 20% of the total thickness) of the corneal stroma. After the corneal inlay is inserted in the pocket, the anterior cornea lies over the corneal inlay and creates a new refractive power for the eye without making a LASIK flap. In these embodiments, this cutting by the femtosecond laser and removal of the cut tissue is followed with intrastromal crosslinking using riboflavin and ultraviolet (UV) radiation. Advantageously, the above-described illustrative embodiment involving the formation of a corneal pocket does not cut many corneal nerves under the surface of the cornea as that which happens with a formation of a LASIK flap. As such, the above-described illustrative embodiment prevents corneal anesthesia and dry eye formation as is the case with a standard LASIK procedure. In addition, the patient advantageously can see the outside world immediately because the central part of the inlay is removed, and the anterior cornea contacts the posterior host cornea. In a situation where the inlay is placed over the corneal stroma after formation of a LASIK flap, it takes about a month or more for the corneal inlay to become as transparent as the rest of the cornea.

In the illustrative embodiment of FIGS. 91 and 92, a corneal inlay 934 with a pinhole 938 for the treatment of hyperopia or presbyopia is depicted. When a patient has hyperopia, the cornea is too flat, which creates a focal point behind the retina. A patient with hyperopia needs a correction that makes his or her cornea more convex. In this embodiment, the pinhole 938 of the inlay 934 may have a diameter between 1 to 3 millimeters, and the inlay 934 may be prepared for implantation by forming an annular concave recess 940 surrounding the pinhole 938, thereby creating a convex central portion of the inlay 934 with the pinhole 938. In the illustrative embodiment of FIG. 93, a corneal inlay 934 is shown implanted between the anterior cornea and the posterior cornea. The corneal inlay 934 of FIG. 93 has a central pinhole 938 surrounded by a darkened peripheral wall 936. The corneal inlay 934 of FIG. 93 also has a central ablation (e.g., an annular concave recess 940 surrounding the pinhole 938) that may be cut using a femtosecond laser and removed. The corneal inlay 934 advantageously improves the stability of the cornea 902 of a patient by increasing the corneal thickness of the patient. Also, the crosslinking of the corneal inlay and stroma further increases the stability of the cornea of the patient, and additionally kills any pathogens that have come into contact with the corneal inlay.

In one or more embodiments, a polycarbonate aliphatic biodurable thermoplastic polyurethane elastomer (e.g., ChronoFlex®) or other suitable polymers are used to create a pinhole for the corneal inlay (e.g., the central pinhole 938 surrounded by a darkened peripheral wall 936 in the corneal inlay 934 of the FIGS. 91-93).

In one embodiment, the corneal inlay made from donor tissue is sterilized by a medication that is approved by the FDA, and is used to increase mechanical stability of the cornea using a solution of riboflavin (a vitamin) in which the corneal inlay is placed and then radiated with UVA light or laser to crosslink the inlay, collagen, and kill simultaneously the bacteria, and viruses, etc., while crosslinking the donor cells, etc. in the corneal inlay and preventing an immune response to the donor tissue of the inlay.

In one embodiment, the sterilized corneal inlay is placed over the exposed corneal stroma and ablated with an excimer laser or a femtosecond laser to remove an amount of tissue needed for the correction of the refractive error of the eye and simultaneously remove about 10-20 micron thickness or more from the host stromal thickness, to create a hole in the corneal inlay (e.g., see FIGS. 82A, 82B, 84, 85, and 90).

In another embodiment, a ring of the corneal inlay is made of ChronoFlex® and carbon black or another polymer with a thickness of 20-50 microns or more and a diameter of 2.2 mm to be placed inside a central space created for the pinhole inside the corneal inlay.

In one embodiment, the central ring of the corneal inlay has a diameter of about 1-2 mm, or preferably 1.4 mm, and after correction of the refractive error of the eye using the corneal inlay with an excimer laser or a femtosecond laser where a center of the inlay and a 10-20 micron thick tissue of the host corneal stroma is removed with the laser, then the thin polymeric ring and carbon black is placed inside the center of the corneal inlay, then the corneal flap which was put aside initially is repositioned over the implanted corneal inlay with its pinhole. Because this procedure removes any polymeric material or tissue from the central pinhole, the tissue of the initial corneal flap comes directly in contact with the host corneal stroma, below the inlay so that the pinhole is not covered with the transplanted donor tissue. There will not be any polymeric or swollen donor tissue in the central opening of the pinhole. Therefore, the patient can immediately regain his vision through the pinhole thereby seeing the objects in the outside world immediately clear after the aforedescribed Tissue Augmented LASIK (TAL) procedure.

In one embodiment, the crosslinking of the inlay can be done outside the body before implantation or after its implantation inside the cornea, using riboflavin which is injected over the implanted inlay and UVA radiated from the outside of the eye (i.e., outside the cornea). The implantation of the pinhole inlay is done so that the center of the pinhole coincides with the center of the cornea, at the corneal axis over the pupil or under the angle Kappa or half way between the pupil and angle Kappa.

In one embodiment, the ring structure can be coated with a thin layer (e.g., about 5 nm) of plasma polymer of methane (plasma polymerization) to seal the entire ring polymer or intraocular lens, preventing the leakage of molecules, such as silver nanoparticles, from the pinhole structure, while maintaining the oxygen permeability for the corneal inlay, intraocular lens (IOL), contact lenses, or the scleral lenses, etc.

In one embodiment, after corneal implantation and ablation, one applies two or more drops of about 1% or more riboflavin solution or another photosensitizer for crosslinking with or without Rock inhibitors with or without allopregnanolone at <8 nanograms/ml to encourage nerve growth, over the inlay which is then covered immediately with the corneal flap, permitting for 1-5 minutes, the riboflavin to penetrate the inlay and the surrounding stroma which are then crosslinked with UV radiation of 3-10 milliW/cm2 or more from the outside of the eye through the cornea for 1-10 minutes or more to crosslink the inlay and its surrounding tissue and kill all pathogens by UV and crosslink the inlay and sterilize the corneal cavity and increase the mechanical stability of the cornea after its crosslinking (see FIG. 47B).

In one embodiment, in the postoperative period, the cornea is treated with a solution or ointment of medications such as a Rock inhibitor, Wnt inhibitors, GSK inhibitors, or anti-integrin alone or with steroids or NSAIDs or a nerve growth factor to encourage nerve growth in the previously cut corneal tissue and reduce inflammation. Since the corneal flap is mostly not crosslinked because the photosensitizer will be limited to the inlay and surrounding tissue, the growth of the corneal nerve recovery in the corneal flap that is not crosslinked is enhanced, using the above-mentioned mediations or with addition of a steroid, NSAIDs or an antibiotic or antiviral, etc. after surgery. This can also enhance recovery and the wound healing of the corneal nerves and return of the corneal sensation, thereby preventing dry eye after surgery.

In one embodiment, in the postoperative period, the cornea is treated with a solution or ointment of medications such as Rock inhibitors, Wnt inhibitors, GSK inhibitors, or anti-integrin alone or with steroids or anti-inflammatory agents, with or without complement pathway inhibitors, such as C3 inhibitors—AMY-101 (NCT04395456) and APL-9 (NCT04402060); C5 inhibitors—eculizumab (NCT04346797 and NCT04355494), C1 esterase inhibitors, which block the classical complement pathway with anti-IL6 medication, such as Kevzara, tocilizumab, rituximab, etc. and antibiotics, such as tetracycline derivatives, a metalloproteinase inhibitor and/or low molecular weight heparin (Lovenox), and/or in a suitable medium to enhance nerve growth after refractive surgery.

In one embodiment, after refractive surgery, the cornea is treated with a solution or polymeric nanoparticles PGLA and a Rock inhibitor, alone or with steroids or with a complement inhibitor, such as anti-IL6 medication Kevzara, in a medium of low molecular weight heparin (Lovenox) with or without an antibiotic.

In one embodiment, a Rock inhibitor or another cell inflammatory pathway inhibitor in a semifluorinated alkane combined with a complement inhibitor, such as Kevzara, and or an anti-VEGF, such as bevacizumab and/or low molecular weight heparin (LMWH), such as Lovenox etc., is administered intravitreal or topically to the cornea to enhance nerve growth and prevent cell loss caused by chronic inflammation or in an aging eye.

In one embodiment, the corneal inlay or an inlay with the shape of doughnut can be treated or crosslinked ex-vivo and the refractive error of the patient's eye is corrected on the inlay with or without a 1-3 mm central hole prior to implantation, the inner wall of the doughnut hole is darkened with a dye and it is implanted in a corneal pocket, such as a pocket made with a femtosecond laser in the corneal stroma and a small incision is made to implant the doughnut inlay in the corneal pocket (see FIGS. 48A-48C), or the corrected inlay can be implanted under a LASIK flap.

In the embodiment of FIGS. 48A-48C, a corneal pocket/cavity 624 is initially prepared using a femtosecond laser in a host eye 620 with a cornea 622, an iris 626, and a lens 628. Then, an excimer laser is used to correct the refractive error of the eye after an inlay 630 with a central hole 632 (e.g., a hole that is 2-3 mm in diameter) surrounded by a darkened central wall is implanted in the corneal pocket/cavity 624. Then, one or more drops of a photosensitizer, such as riboflavin, are injected into the pocket/cavity 624, and the eye 620 is irradiated with an ultraviolet (UV) laser from outside so as to crosslink the inlay 630 and surrounding tissue and to sterilize the inlay 630 and the corneal pocket/cavity 624. As such, the corneal inlay 630 corrects the refractive errors of the eye 620 and presbyopia.

In one embodiment, one uses a femtosecond laser to create a central corneal cavity in a cornea with keratoconus from 7-10 mm in diameter, the corneal tissue is separated with a spatula, then a corneal inlay is created with a diameter of 7-9 mm and a central hole of 1-3 mm and a thickness as needed to ablate the cornea. The inner wall of the central hole is darkened with a dye or carbon nanoparticles etc., leaving a peripheral area clear, thereby creating an inlay that can compensate for most to all of the refractive error of the cornea, and presbyopia then the inlay with the central hole (doughnut) is crosslinked with riboflavin and UV radiation prior to its implantation or after implantation. The inlay is covered with hyaluronic acid or a viscoelastic material and injected inside the corneal cavity. Other medications, such as inflammatory pathway inhibitors with or without steroids or antibiotics, are injected either inside the corneal cavity or applied postoperatively to prevent an infection, reduce inflammation and enhance the nerve regeneration.

In one embodiment, this technology with or without modification can be employed to treat refractive error and presbyopia.

In one embodiment, the use of animal inlays, decellularization and riboflavin crosslinking of the inlay eliminate the antigenic proteins in the inlay regardless of the origin of the inlay and makes them less immunogenic in the host tissue. The composition of the inlay should have <30%-50% water content to prevent swelling of the inlay. The eye should be treated with a combination of a Rock inhibitor, steroids or adalimumab, etc. to suppress an immune response.

In one embodiment, the TA LASIK can be done with a femtosecond laser to remove the desired tissue from the inlay and the host cornea, the central part is removed carefully so as to leave the remaining inlay tissue and a central hole in the inlay, followed by crosslinking the inlay and the wall of the stromal tissue before or after the flap is replaced over the inlay.

In one embodiment, the riboflavin crosslinking can be repeated as needed to denature the corneal tissue around the inlay.

In one embodiment, the cell inflammatory pathway inhibitors can be combined with anti-inflammatory agents such as doxycycline, or biologics such as adalimumab, anti-VEGFs, antibiotics or complement inhibitors etc. applied to the cornea or conjunctiva after the surgery to suppress inflammation, while encouraging nerve growth in the tissue and prevent an infection.

In one embodiment for correcting the refractive error of the cornea or presbyopia, one creates a LASIK flap 644 (see e.g., FIGS. 49-51) using a femtosecond laser or a microkeratome in one eye or both eyes of the patient or an animal, where the diameter of the corneal flap 644 is about 4-8 mm or more, the flap 644 is pushed to one side, and a human, animal, or a synthetic 3-D printed corneal inlay 648, which is decellularized by medication or by any form of radiation and/or crosslinked with UV radiation and riboflavin or other photosensitizers and laser wavelengths before or after implantation, is placed on the corneal stroma 646. The corneal inlay 648 has the desired diameter and thickness to be ablated with an excimer laser 652 to correct the refractive error of the eye in myopia, hyperopia, astigmatism, using a Shack-Hartmann sensor to correct astigmatism or presbyopia, then a central through hole 650 of 1-3 mm is cut in its center as needed prior to the implantation or the inlay 648 is placed over the corneal stroma 646 after its implantation and the central hole 650 is cut away with a femtosecond laser. The corneal flap 644 can be positioned over the corneal inlay 648 and corneal crosslinking is done either before or after implantation to prevent rejection of the inlay 648.

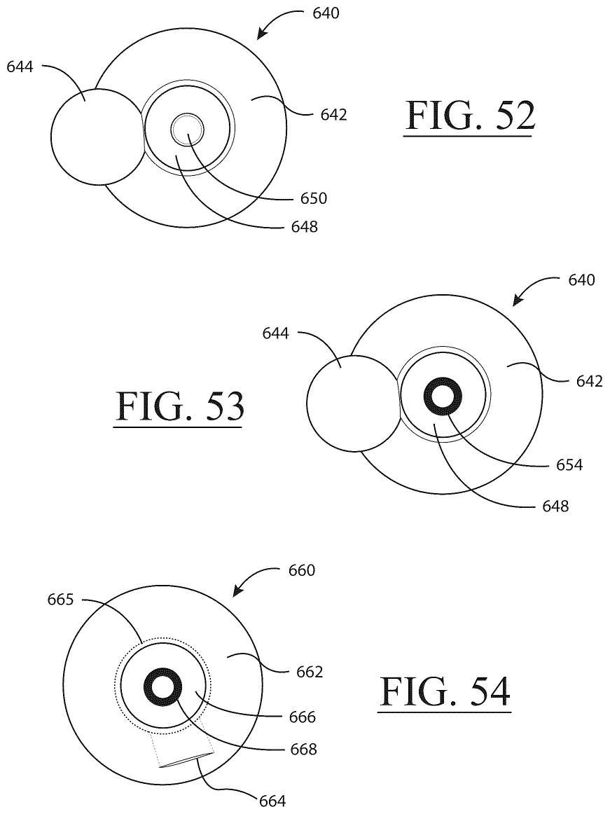

In one embodiment, referring to FIGS. 52 and 53, prior to repositioning of the corneal flap 644, a polymeric pinhole 654 made of polyvinylidene fluoride (PVDF) carbon black or poly(methyl methacrylate) (PMMA) carbon black or another polymer is placed inside the central hole 650 of the inlay 648, then the flap 644 is repositioned over the inlay 648, then the cornea 642 and inlay 648 can be crosslinked with riboflavin solution and UV radiation, etc. to prevent rejection of the inlay 648 or the polymeric pinhole 654.

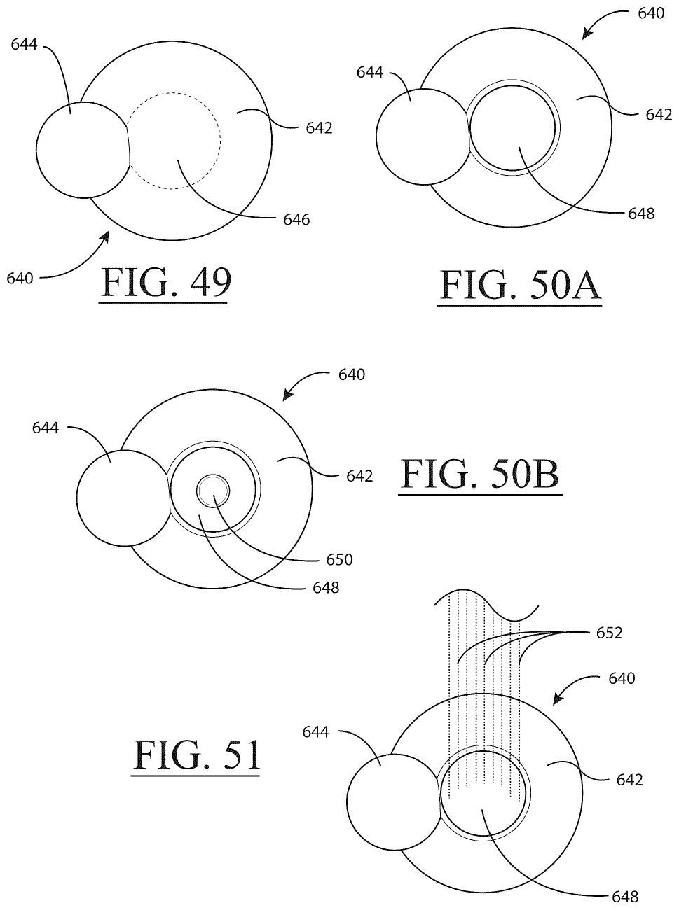

In one embodiment, with reference initially to FIG. 49, it can be seen that a corneal flap 644, which has been formed in the cornea 642 of an eye 640, has been rotated to expose stromal tissue 646 of the cornea 642 underneath the flap 644. Then, in FIG. 50A, the corneal inlay 648 has been placed on the exposed stromal tissue 646 of the cornea 642 of the eye 640. After which, as shown in FIG. 50B, a central hole 650 may be formed in the corneal inlay 648. Also, as depicted in FIG. 51, an excimer laser 652 may be used to ablate the corneal inlay 648 placed on the exposed stromal tissue 646 to correct the refractive error of the eye 640. In FIG. 52, a central hole 650 has been formed in the corneal inlay 648 using a trephine. Then, in FIG. 53, a pinhole ring 654 with darkened peripheral wall has been inserted into the hole 650 of the corneal inlay 648. The corneal inlay 648 in FIG. 53 has been irradiated with an excimer laser to correct the refractive error of the eye 640. In the embodiment of FIG. 53, the corneal flap 644 may have a diameter of 6 to 7 millimeters (mm), the pinhole ring 654 may have an outer diameter of 2.2 to 2.4 mm, and the central pinhole in the pinhole ring 654 may have a diameter of 1 to 2 mm.

In one embodiment, the corneal inlay with a central hole is corrected with an excimer laser then placed inside the corneal without removing any tissue and subsequently a polymeric pinhole with darkened wall is inserted in the central hole of the corneal inlay.

In another embodiment, with reference to the eye 660 depicted in FIG. 54, a thin 3-D circular part of a corneal stroma is removed to make a corneal pocket 665 in a cornea 662 and replaced with an inlay 666 with a central hole that has been corrected with an excimer laser. The inlay 666 is implanted in the corneal pocket 665 with a viscoelastic solution and subsequently a polymeric dark pinhole 668 made of polyvinylidene fluoride (PVDF) carbon black or poly(methyl methacrylate) (PMMA) carbon black is implanted through the same corneal stromal incision 664 inside the inlay's hole followed with or without corneal crosslinking.

In one embodiment, to address conditions occurring in the elderly, a surgical procedure has been developed that permits correction of the refractive error for any age group without thinning the cornea in order to treat both the presbyopia and refractive error of the eye regardless of the degree of corneal thickness. The corneal inlay is modified as described by making a corneal flap, pushing the flap to one side, placing a desired corneal inlay with a desired thickness and diameter over the center of the cornea, correcting initially the refractive error of the eye, if needed, with an excimer laser, and then using a trephine of desired diameter of 1-3 mm or more on the center of corneal inlay. A circular area of 1-3 mm of the inlay is initially removed, but replaced with a non-toxic circular polymeric pinhole made of poly(methyl methacrylate) (PMMA) or polyvinylidene fluoride (PVDF) mixed with carbon black in which a central hole with a diameter of 1-3 mm was created, and the polymeric pinhole with a central hole is placed inside the circular inlay (see FIGS. 52 and 53) to create a pinhole with a dark peripheral part, then the initial LASIK flap is repositioned over the entire inlay that has the synthetic non-toxic pinhole made of a polymer and carbon black. The LASIK flap can be subsequently crosslinked with UV radiation and riboflavin before or after the LASIK flap is replaced (see FIGS. 53 and 54) and appropriate medications, such as topical steroids, antibiotics, or nerve growth stimulators, etc. are applied to the cornea. The creation of a pinhole as described permits the patient to see in focus objects located at a far, intermediate distance or near at any distance in between, while the peripheral part of the corneal inlay corrects the remaining refractive error of the eyes beyond the central area.

In one embodiment, after the above procedure has been performed, the cataract extraction can be performed if needed, through a small 2-3 mm incision made in the peripheral cornea into the anterior chamber of the eye to remove the anterior 5-6 mm of the lens capsule through which the cataract is removed using a phacoemulsification and irrigation and implantation of an appropriate intraocular lens, the central corneal pinhole permits seeing an object at any distance from the eye. Since the light that passes through the pinhole is always in focus on the retina and it does not bend as is the case with the crystalline lens or implanted intraocular lenses.

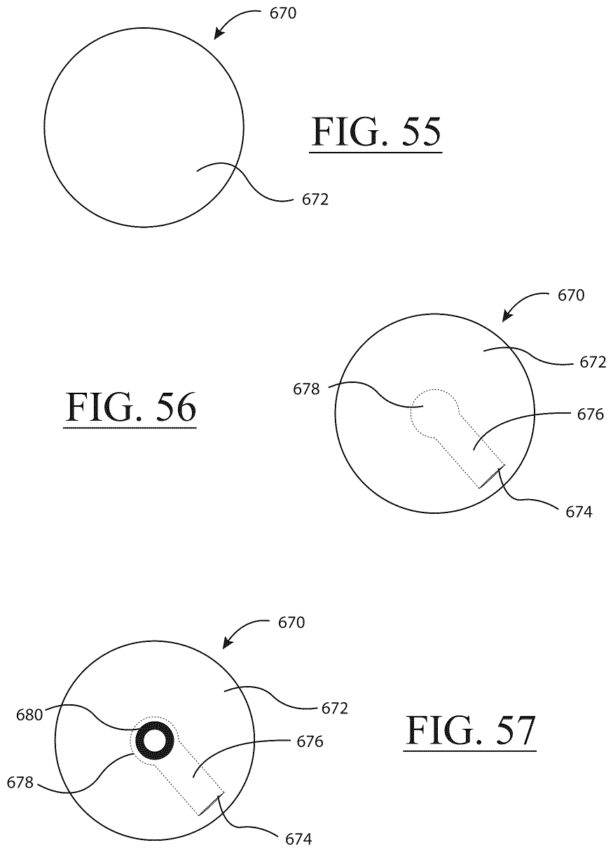

In one alternative embodiment, in elderly patients having presbyopia with difficulty seeing near objects, one can be treated by making a small intrastromal channel 676 in the peripheral corneal stroma of the eye 670 through which a corneal pocket 678 or pathway pocket is created using a knife with a semicircular tip or a femtosecond laser (refer to FIGS. 55 and 56). As shown in FIG. 56, an incision 674 is initially formed in the cornea 672 of the eye 670, and then the intrastromal channel 676 and corneal pocket 678 are formed in the cornea 672. After which, referring to FIG. 57, a non-toxic poly(methyl methacrylate) (PMMA) carbon black or polyvinylidene fluoride (PVDF) carbon black (CB) pinhole ring 680 in a circular configuration having a diameter of 1-4 mm and a central hole of 1-3 mm and a thickness of 10 to 30 microns or more is placed inside the corneal pocket 678 along with viscoelastics for near-frictionless implantation where the central 1-3 mm tissue is removed, in one or both eyes of the patient or after a LASIK or a Smile procedure, or PRK surgery. This procedure permits the eyes to see far or near, or an intermediate distance. This procedure permits any child, or young or old person, a person with myopia, or hyperopia or astigmatism, or children with strabismus to see in focus with both eyes and prevent amblyopia (loss of sight) to develop or treat them if the amblyopia has developed, since amblyopia and myopia develops in children when their eyes cannot see the object far or near in focus, then they use the dominant eye only, whereas the implanted polymeric plus carbon black pinhole provides an in focus image for the retina regardless of the dioptric error of the eyes, thereby permitting simultaneous in focus images for both eyes.

In one embodiment, this procedure prevents uncontrolled growth of the eye in children having hyperopia or myopia, and thus, makes these eyes less prone to getting a retinal tear or retinal detachment, etc.

In one embodiment, the polymeric pinhole can be made from other non-toxic polymers in combination with carbon black, polymethyl methacrylate or polyvinylidene or another non-toxic polymers plus carbon black molded or 3-D printed having the described design.

In one embodiment, the pinhole in the center of a polymer carbon black composition looks like a flat ring structure and its circular peripheral part has invisible micro holes made by exposing the polymer's surface to a laser beam, such as a femtosecond laser, in an oblique fashion, applied to the polymeric portion of the pinhole for water molecules to pass through the channels, while light coming perpendicular to the dark wall of the pinhole will be absorbed by the carbon black. This modification permits the water to pass through the micro-holes and keep the corneal stroma moist.

In one embodiment, polyvinylidene fluoride (PVDF) is used as a polymer with carbon black to make the circular pinhole. Polyvinylidene fluoride (PVDF), also known as polyvinylidene, is a thermoplastic polymer which is FDA-compliant and non-toxic in the body below its melting temperature. PVDF is used in biomedical food, beverage processing, and as an ultrapure compound for applications in water management. This polymer has piezoelectric properties and is used in the manufacturing of various high-strength, high-purity chemical-resistance containers. PVDF has high thermal stability (>92-342° C.) and resists UV radiation with minimal water absorption. PVDF is non-toxic and resists fungi and bacteria. PVDF is used commonly by 3-D printing technology.

In one embodiment, carbon black can be mixed with various non-toxic polymers to change their pigmentation or serve as a light absorber at visible and non-visible wavelengths or a conductive filler. Other polymers which can be used in making polymeric pinhole are: Polycarbonate (PC) PMMA or Acrylic, Fluorinated Ethylene Propylene (FEP Transparent Polypropylene (PP) Polyethylene Terephthalate (PET) Polyvinyl Chloride (PVC) Styrene Methyl Methacrylate (SMMA) Styrene Acrylonitrile Resin (SAN) Polystyrene (General Purpose-GPPS) or MABS (Transparent ABS) Biocompatible plastics such as PVC and Polyethylene, PEEK, Polycarbonate, Ultem PEI, Polysulfone, Polypropylene and Polyurethane, Silicon, Polyacrylonitrile/Carbon Black nanoparticle.

Polyvinylidene fluoride (PVDF) carbon black or poly(methyl methacrylate) (PMMA)/carbon black (CB) or other polymer composites, e.g., a polymer and carbon black powder or modified carbon black are fabricated using different mixing methods: (i) solution mixing of the precursors and compression molding, (ii) mechanical mixing, or (iii) 3-D printing to add and mix carbon black at any stage of forming the structure, such as an optical lens or a pinhole inside the cornea or a virtual pinhole where the center of the hole inside the polymeric ring of carbon polymer is filled with the transparent polymer again creating a virtual pinhole.

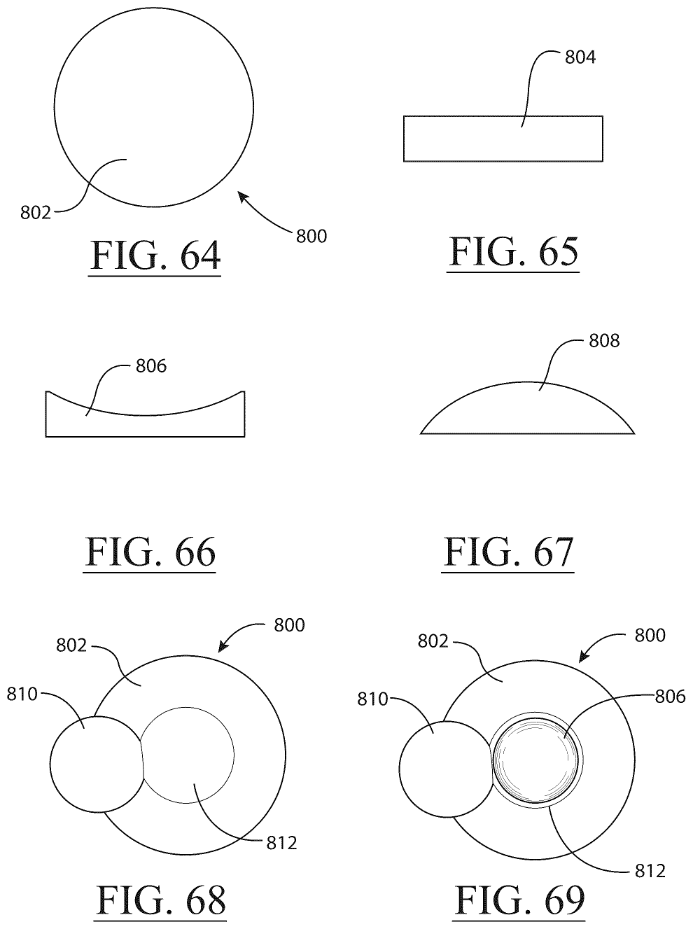

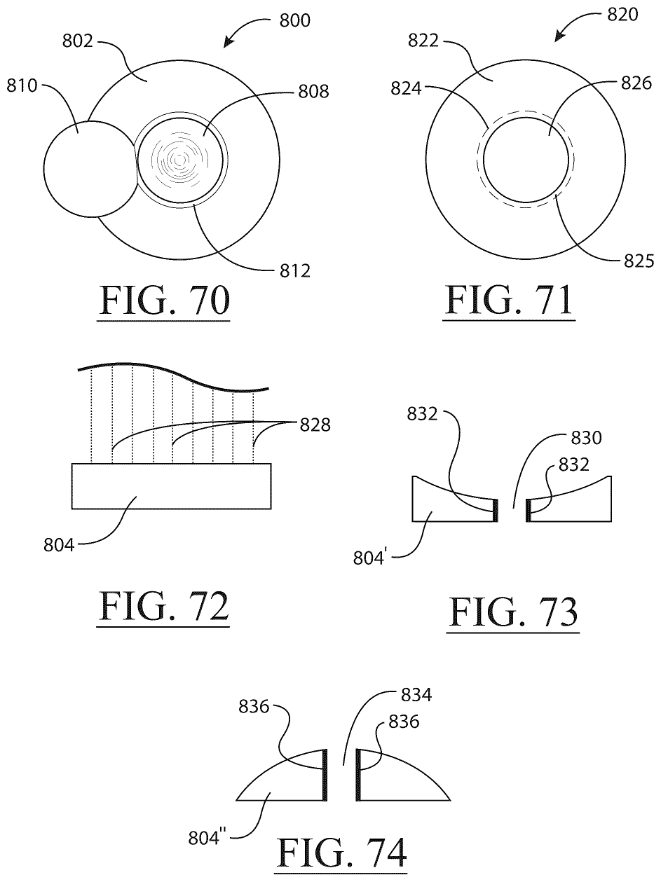

In one embodiment for correcting the refractive error of the cornea and/or presbyopia of an eye of a patient, with reference initially to FIGS. 64 and 68, it can be seen that a corneal flap 810 (e.g., a LASIK flap), which has been formed in the cornea 802 of an eye 800, has been rotated to expose stromal tissue 812 of the cornea 802 underneath the flap 810. Then, in FIG. 69, a corneal inlay 806 (e.g., the molded concave corneal inlay 806 depicted in FIG. 66) has been placed on the exposed stromal tissue 812 of the cornea 802 of the eye 800. Alternatively, in FIG. 70, a corneal inlay 808 having a different shape (e.g., the molded convex corneal inlay 808 depicted in FIG. 67) has been placed on the exposed stromal tissue 812 of the cornea 802 of the eye 800. Then, a photosensitizer (e.g., riboflavin) may be applied to the cornea 802 of the eye 800 of the patient so that the photosensitizer permeates at least a portion of the corneal tissue surrounding the corneal inlay 806, 808 and/or at least a portion of the corneal inlay 806, 808, and the corneal flap 810 is repositioned back over the corneal inlay 806, 808 to absorb the photosensitizer. After the photosensitizer is applied and the corneal flap 810 is closed, the cornea 802 may be irradiated so as to activate cross-linkers in the corneal inlay 806, 808 and/or cross-linkers in the portion of the host corneal tissue surrounding the corneal inlay 806, 808, and thereby prevent an immune response from the patient, prevent rejection of the corneal inlay 806, 808 by the patient, and/or strengthen the biomechanical properties of the corneal inlay 806, 808. In this embodiment, the corneal inlay 806, 808 may be configured to simultaneously correct refractive errors and presbyopia in the eye 800 of the patient.

In another embodiment, with reference to the eye 820 depicted in FIG. 71, a thin 3-D circular part of a corneal stroma is removed to make a corneal pocket 824 with a pocket cavity 825 in a cornea 822, and replaced with a corneal inlay 826. The corneal inlay 826 is implanted in the cavity 825 of the corneal pocket 824 with a viscoelastic solution.

Now, referring to FIGS. 72-74, formation of corneal inlays having various shapes will be described. Initially, as shown in FIG. 72, the corneal inlay 804 depicted in FIG. 65 is shown being ablated using a laser 828 (e.g., an excimer laser) in order to shape corneal inlay 804 for correcting a particular refractive error and/or presbyopia. In FIG. 73, a concave corneal inlay 804′ that has been formed from the corneal inlay 804 of FIG. 72 by ablation is depicted. As shown in FIG. 73, the concave corneal inlay 804′ comprises a central pinhole 830 disposed therethrough, which is surrounded by a darkened wall 832 that has been formed in the corneal inlay 804′. In FIG. 74, a convex corneal inlay 804″ that has been formed from the corneal inlay 804 of FIG. 72 by ablation is depicted. As shown in FIG. 74, the convex corneal inlay 804″ comprises a central pinhole 834 disposed therethrough, which is surrounded by a darkened wall 836 that has been formed in the corneal inlay 804″.

In a further embodiment, in an early stage of keratoconus disease, the treatment is done by glasses or contact lenses which are positioned in front of the eye as glasses or over the cornea as contact lenses.

In one embodiment, in the early stages of keratoconus, one can stabilize the cornea by crosslinking its collagenous component of the cornea with a procedure called corneal crosslinking in which a solution containing a crosslinker photosensitizer, such as riboflavin, etc., at concentrations of 0.1-1% or more that is applied to the cornea for a time of 5 minutes or more depending on the concentration of the photosensitizer, then at least one-half of the corneal thickness is flooded using a UV laser at powers of 1-10 milliwatt/cm2 depending on the concentration of the photosensitizer for a period of 3-30 minutes to crosslink the cornea and stabilize the biomechanics of the cornea to resist the intraocular pressure and prevent expansion of the cornea.

In one embodiment, the corneal expansion leads to the thinning of the corneal thickness, which is in general about 550 microns with production of a round thin dome near the center of the cornea.

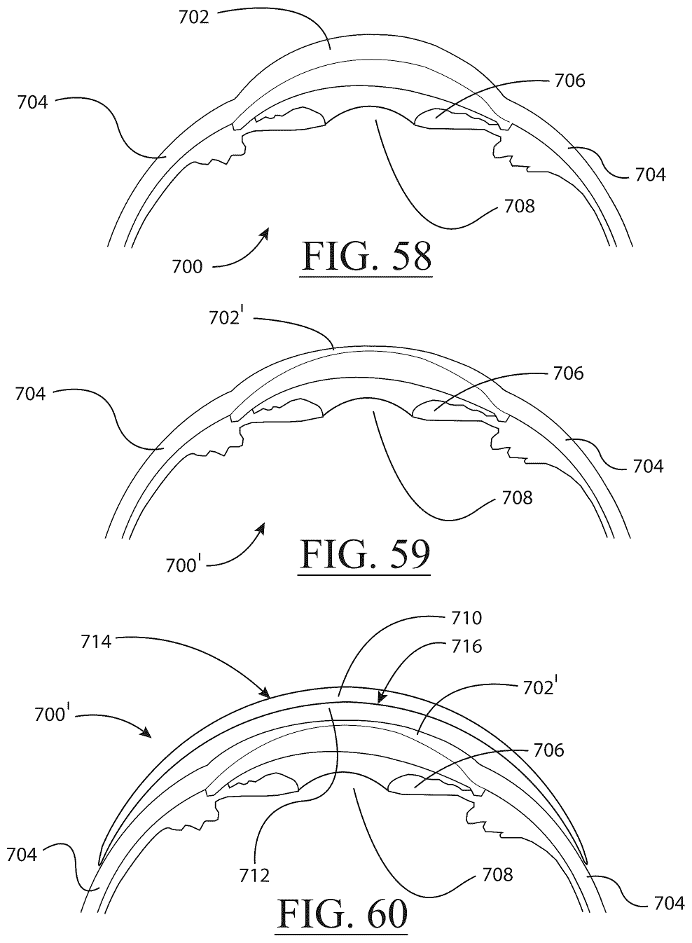

In one embodiment, progressing keratoconus requires scleral contact lenses that are not directly located on the surface of the cornea as regular contact lenses, but rather beyond the cornea, and on the sclera, over the conjunctiva, thus the scleral contact lenses produce a space between the anterior corneal surface and the scleral lens, where the space is filled with a fluid such as a physiological saline solution, etc. (refer to FIGS. 58-60). Therefore, the refractive power of the cornea is eliminated by presence of liquid on both sides of the cornea (i.e., the anterior chamber and the liquid reservoir created in front of the cornea and the scleral lens-see FIG. 60). As a result, the corneal curvature and its refractive power is defined by the anterior surface of the scleral lens and its relationship with the posterior surface of the scleral lens that is in contact with the reservoir fluid.

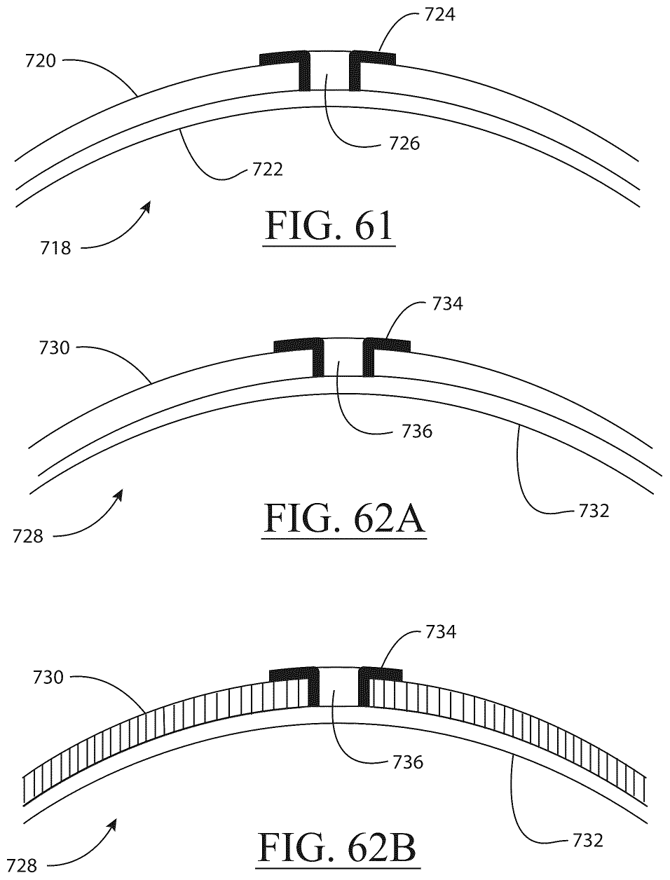

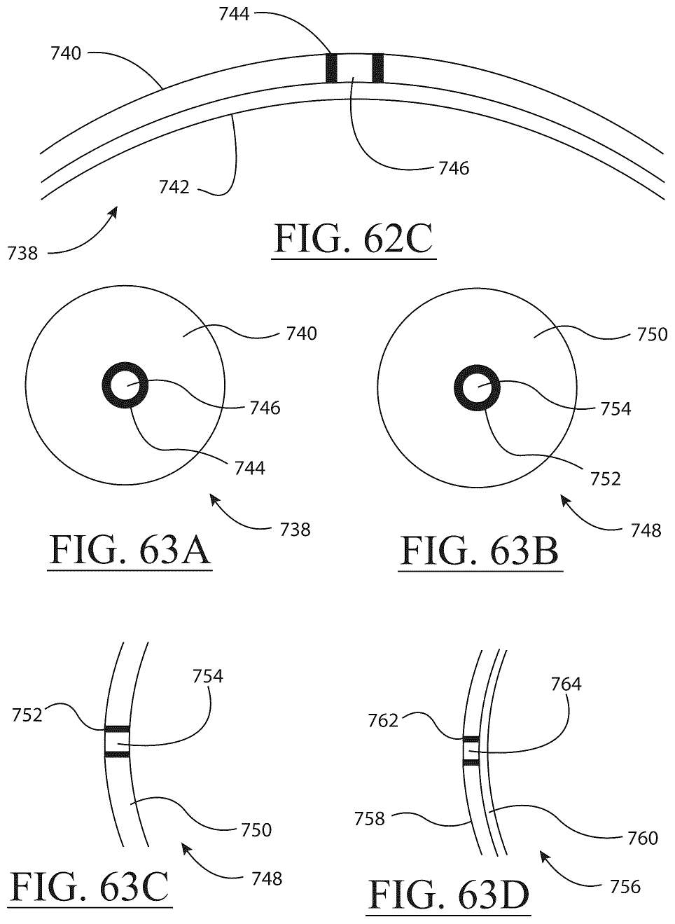

In FIG. 58, an eye 700 with a normal cornea 702, a sclera 704, an iris 706, and a pupil 708 is illustrated. In FIG. 59, an eye 700′ with a thinned cornea 702′ due to keratoconus, a sclera 704, an iris 706, and a pupil 708 is illustrated. In FIG. 60, a scleral lens 710 is shown disposed over the thinned cornea 702′ of the eye 700′. A fluid or liquid reservoir 712 is disposed between the scleral lens 710 and the thinned cornea 702′. The scleral lens 710 may have inner and outer layers as described hereinafter with respect to FIGS. 61 and 62A-62C. When provided with two layers, a soft internal polymer layer of the scleral lens 710 provides an inner refractive correction 716 for the patient, and a rigid external polymer layer of the scleral lens 710 provides an outer refractive correction 714 for treating presbyopia.

In one embodiment with a scleral contact lens, one can apply the photosensitizer at a desired concentration to the fluid in the reservoir in front of the cornea or by applying it topically on the eye to migrate into the liquid reservoir, and then applying UV radiation at the desired power to the crosslinker to the anterior part of the cornea to crosslink a part of the deformed cornea and prevent or slow down an expansion of the cornea.

In one embodiment, in a patient having keratoconus, one can apply riboflavin or another photosensitizer to the eye having keratoconus or myopia so that in sunlight, the potential conjunctiva or meibomian glands bacteria can be damaged by the sunlight for a short period of time.

In one embodiment, a scleral lens can serve as a reservoir for drug delivery with or without existence of keratoconus, not only to the cornea and anterior chamber, but using nanoparticles, the medication present in the reservoir fluid after its topical application can penetrate the entire eye up to the retina for treatment of corneal diseases, infections, inflammation, glaucoma, postoperative inflammation, infection or retinal diseases, such as diabetic retinopathy, or wet or dry form of age-related macular degeneration, viral bacterial infections, or immunomodulators can be applied daily to the eye of the patient, or can be applied as long lasting polymeric nanoparticles.

In one embodiment, the scleral contact lens is made from two parts, namely a soft internal polymer and a rigid external polymer with a central through hole reaching the anterior surface of the internal polymer in which a synthetic pin with a darkened wall and clear transparent center is positioned in the external opening of the scleral lens (refer to FIGS. 61 and FIGS. 62A-62C) that closes the opening and acts as a pinhole with a diameter of 1-2 mm or more with the darkened wall that permits the light passing through it centrally, thus acting as a pinhole transparent plug, and through which all images from near and far are in focus on the retina for a far-sighted or near-sighted patient or elderly patient with presbyopia with or without intraocular lenses.

In FIG. 62C, a scleral lens 738 having a non-flanged pinhole plug or pin 744 is depicted. As shown in this figure, the scleral lens 738 has a lens body formed by a rigid external polymer layer 740 and a soft internal polymer layer 742. Also, as shown in FIG. 62C, the non-flanged pinhole plug or pin 744 of the scleral lens 738 extends through the rigid external polymer layer 740 of the lens body, but not through the soft internal polymer 742 of the lens body so that the interior surface of the lens body is solid. In FIG. 62C, the non-flanged pinhole plug or pin 744 has a transparent center 746. Unlike the light passing through the peripheral part of the scleral lens 738, the light passing through the transparent center 746 of the pinhole plug or pin 744 is not refracted at all. A front view of the scleral lens 738 is depicted in FIG. 63A.

In one embodiment, the lens body of the scleral lens includes a central virtual pinhole that is formed either by the darkening of the lens polymer material in a central annular region, or by means of a synthetic plug or pin with a darkened wall and clear transparent center that is inserted into a central aperture disposed through the lens body. When the synthetic plug or pin 724 forms the central virtual pinhole of the lens body of the scleral lens 718, the central aperture in the lens body that receives the synthetic plug or pin 724 may extend through the rigid external polymer 720 of the lens body, but the soft internal polymer 722 of the lens body may be continuous across the central aperture so that the interior surface of the lens body is solid (see e.g., FIG. 61). In FIG. 61, the synthetic pinhole plug or pin 724 has a transparent center 726.

In one embodiment, the lens body of the scleral contact lens is formed from a single or double transparent soft and rigid oxygen permeable polymer material, and the central pinhole is formed by a circular dark mask placed on either the front or the back central surface of the lens that is stained with dark polymer material or a light impermeable material.

In one embodiment, the rigid part of the polymeric scleral lens with the darkened pinhole (see FIGS. 61-63C), in addition to being rigid oxygen permeable (ROP) or rigid gas-permeable (RGP), contains light sensitive molecules as known in the art that can either block certain wavelengths of light, such as UV, or most of the visible spectrum of light and act as transitional lenses that become dark with the sun exposure, and transparent without the sunlight.

For example, a transitional scleral lens 728 is depicted in FIGS. 62A and 62B. As shown in these figures, the transitional scleral lens 728 has a lens body formed by a rigid external polymer layer 730 with light sensitive molecules and a soft internal polymer layer 732. Also, as shown in FIGS. 62A and 62B, the transitional scleral lens 728 comprises a flanged pinhole plug or pin 734 that extends through the rigid external polymer layer 730 of the lens body, but not through the soft internal polymer 732 of the lens body so that the interior surface of the lens body is solid. In FIGS. 62A and 62B, the flanged pinhole plug or pin 734 has a transparent center 736. In FIG. 62A, the transitional scleral lens 728 is disposed in a dark condition such that the light sensitive molecules in the rigid external polymer 730 are not activated and the rigid external polymer 730 of the lens body is light in color. In FIG. 62B, the transitional scleral lens 728 is disposed in a bright light condition such that the light sensitive molecules in the rigid external polymer 730 are activated and the rigid external polymer 730 of the lens body is dark in color.

In FIGS. 63B and 63C, a scleral lens 748 with a central darkened wall pinhole 752 formed from a double stained polymer is illustrated. In the embodiment of FIGS. 63B and 63C, the lens body 750 of the scleral lens 748 is formed from the double stained polymer, and the central darkened wall pinhole 752 formed from the double stained polymer has a transparent center 754 bounded by a dark circular wall.

FIG. 63D depicts a scleral lens 756 similar to the lens 748 of FIGS. 63B and 63C, except that the lens body of the scleral lens 756 is provided with a soft internal polymer layer 760 in addition to the rigid external polymer 758. In the embodiment of FIG. 63D, the rigid external polymer 758 of the lens body is formed from the double stained polymer, and the central darkened wall pinhole 762 formed from the double stained polymer has a transparent center 764 bounded by a dark circular wall.

In accordance with one or more further embodiments, there is provided a method of correcting refractive errors and presbyopia in an eye of a patient using an ablatable corneal inlay. In one or more of these further embodiments, the patient has keratoconus. The method includes the steps of: (i) forming a darkened annular area of 1-2 mm in a central region of a corneal inlay by tattooing it with a non-toxic dark tattoo material, such as with a liquid black India ink, or polyvinylidene fluoride carbon black mixed with an acrylic polymer so as to define a central pinhole for correcting presbyopia, high myopia, or keratoconus in a host eye of a patient, the darkened annular area being generally non-transparent, while the central part is transparent; (ii) forming a flap or a pocket for receiving the corneal inlay in the cornea of the host eye of the patient; (iii) inserting the corneal inlay into the pocket or on stromal tissue exposed by the flap in the cornea of the host eye of the patient; (iv) modifying the shape of the corneal inlay using an excimer laser or femtosecond laser so that the corneal inlay is capable of correcting refractive errors in the host eye of the patient; (v) applying a photosensitizer to the cornea of the host eye of the patient so that the photosensitizer permeates at least a portion of the host corneal tissue surrounding the corneal inlay and/or at least a portion of the corneal inlay; and (vi) irradiating the cornea with UV radiation so as to activate cross-linkers in the corneal inlay and/or cross-linkers in the portion of the host corneal tissue surrounding the corneal inlay, and thereby prevent an immune response from the patient, while simultaneously killing the pathogenic bacteria, viruses, fungi, or parasites preventing the tattoo material from migrating, and preventing rejection of the corneal inlay by the patient, and/or strengthen the biomechanical properties of the corneal inlay. The corneal inlay is configured to simultaneously correct refractive errors and presbyopia and myopia in the host eye of the patient.

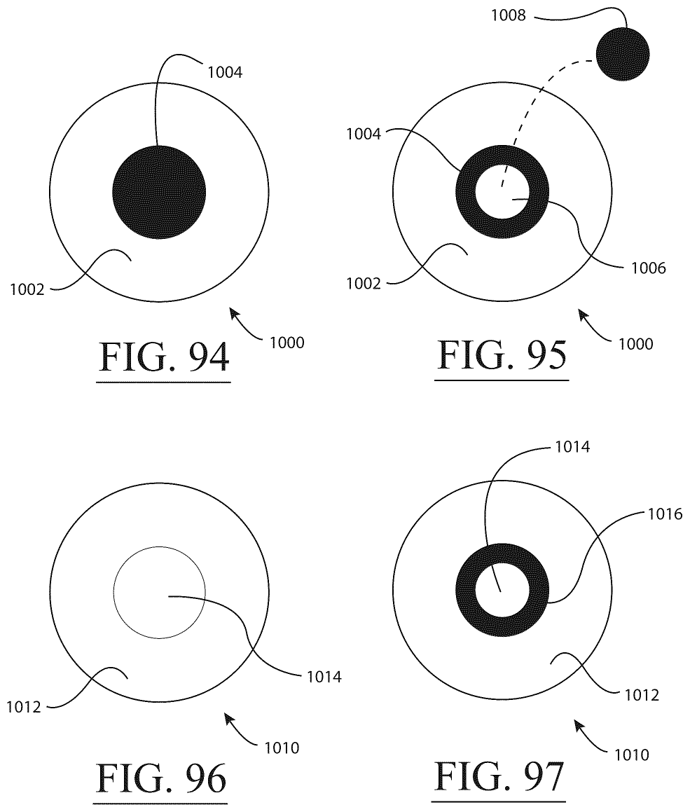

In one embodiment of the corneal inlay 1000, the entire central 1 to 2 mm area 1004 of the inlay body 1002 is tattooed (refer to FIG. 94), but the central 1.4 mm area 1008 or more is removed using a trephine or femtosecond laser leaving a tattooed ring 1004 surrounding a central aperture 1006 through which the light passes unhindered (see FIG. 95). In one or more embodiments, the corneal inlay 1000 depicted in FIGS. 94 and 95 may be formed from natural donor corneal tissue from a human or an animal. Also, in one or more embodiments, the tattooed central area 1004 of the inlay body 1002 is crosslinked so as to kill pathogenic organisms around the central pinhole and extend a length of time that biocompatible, non-toxic tattoo dark or black ink remains embedded in the inlay body 1002 (i.e., the crosslinked cells prevent the biocompatible, non-toxic dark or black ink from escaping outside of the central pinhole area 1004.

In one embodiment, the corneal inlay 1010 with the inlay body 1012 and central 1 to 2 mm pinhole 1014 is implanted in a corneal pocket alone (refer to FIG. 96), or the central pinhole 1014 of the corneal inlay 1010 is filled with a darkened ring 1016 made of a synthetic polyvinylidene fluoride carbon black mixed with an acrylic polymer combination, thereby providing a central pinhole 1014 with a darkened wall 1016 inside the corneal inlay (see FIG. 97). In one or more embodiments, the corneal inlay 1010 depicted in FIGS. 96 and 97 may be formed from natural donor corneal tissue from a human or an animal such that the peripheral portion 1012 of the corneal inlay 1010 circumscribing the darkened ring 1016 is natural tissue, while the darkened ring 1016 is synthetic. Initially, in FIG. 96, the central pinhole aperture 1014 may be formed in the inlay body 1012 of the natural corneal inlay 1010. Then, in FIG. 97, the synthetic darkened ring 1016 is inserted into the central pinhole aperture 1014 of the natural corneal inlay 1010.

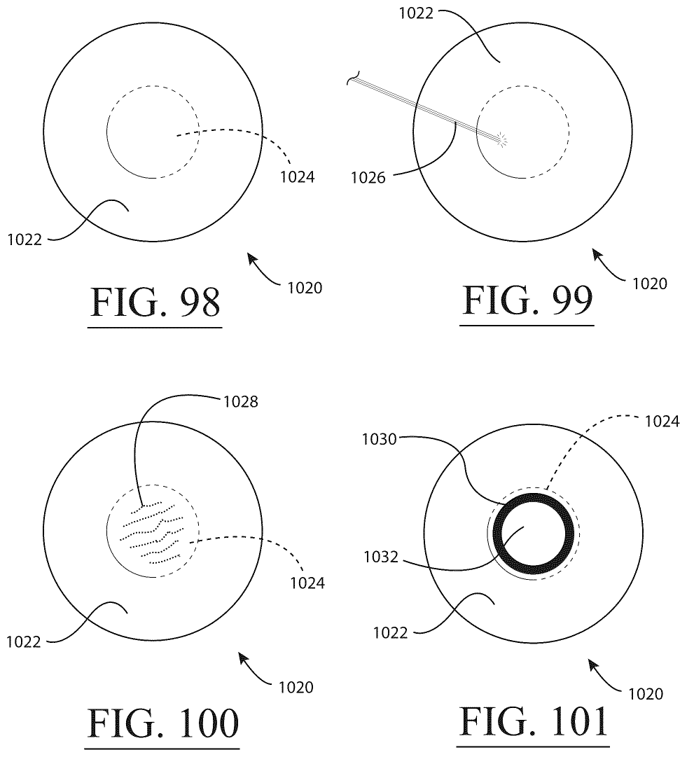

Another embodiment is depicted in FIGS. 98-101. Initially, as shown in FIG. 98, a corneal pocket 1024 with a 2 to 3 mm diameter is formed in corneal tissue 1022 of a cornea 1020 of an eye. Then, in FIG. 99, a femtosecond laser 1026 is used to ablate the tissue inside the corneal pocket 1024 so that the tissue can be removed from the eye. In FIG. 100, the corneal pocket 1024 in the eye is depicted, where the corneal pocket 1024 is disposed in the corneal stroma 1028 under the Bowman's membrane. Finally, in FIG. 101, a ring 1030, which is formed from a synthetic polyvinylidene fluoride carbon black mixed with an acrylic polymer combination, is being inserted into the corneal pocket 1024 in the eye through a small side opening in the corneal pocket 1024. The synthetic ring 1030 has a pinhole 1032 formed in the center thereof with a pinhole diameter of between approximately 0.9 mm and approximately 2.0 mm (or between 0.9 mm and 2.0 mm).

Any of the features, attributes, or steps of the above-described embodiments and variations can be used in combination with any of the other features, attributes, and steps of the above described embodiments and variations as desired.

Although the invention has been shown and described with respect to a certain embodiment or embodiments, it is apparent that this invention can be embodied in many different forms and that many other modifications and variations are possible without departing from the spirit and scope of this invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A corneal inlay for implantation into a cornea of an eye, the corneal inlay comprising:
an inlay body formed from natural tissue of a donor cornea; and
a central pinhole disposed in the inlay body, the central pinhole being surrounded by a darkened bounding wall.

2. The corneal inlay according to claim 1, wherein the darkened bounding wall in the inlay body is formed by initially tattooing a central area of the inlay body using a biocompatible, non-toxic dark or black ink, and then subsequently cutting the central pinhole in the central area of the inlay body using a trephine or a femtosecond laser.

3. The corneal inlay according to claim 2, wherein the tattooed central area of the inlay body is crosslinked so as to kill pathogenic organisms around the central pinhole and extend a length of time that the biocompatible, non-toxic dark or black ink remains embedded in the inlay body.

4. The corneal inlay according to claim 2, wherein the darkened bounding wall is formed by inserting a darkened ring member into an aperture formed in a central area of the inlay body.

5. The corneal inlay according to claim 4, wherein the darkened ring member is formed from polyvinylidene fluoride carbon black mixed with an acrylic polymer.

6. The corneal inlay according to claim 1, wherein the central pinhole of the inlay body is a circular-shaped pinhole, an oval-shaped pinhole, or a slit-shaped pinhole.

7. The corneal inlay according to claim 1, wherein a darkening compound used to form the darkened bounding wall in the inlay body comprises carbon black nanoparticles.

8. A pinhole lens structure, comprising:
an insertable pinhole body defining a through pinhole of approximately 0.9-2.0 mm in diameter with a surrounding darkened wall having an approximately 0.1-1.0 mm wall thickness, the insertable pinhole body configured to be inserted into a corneal pocket in a cornea of an eye.

9. The pinhole lens structure according to claim 8, wherein the corneal pocket has a diameter of approximately 2.0-3.0 mm.

10. The pinhole lens structure according to claim 8, wherein the insertable pinhole body is formed from polyvinylidene fluoride carbon black mixed with an acrylic polymer.
